(19) United States
(12) Patent Application Publication
(10) Pub. No.: US 2026/0124295 A1
(43) Pub. Date: May 7, 2026

(54) USE OF LIPID NANOPARTICLES AND IMMUNE CHECKPOINT INHIBITORS IN THE TREATMENT OF CANCER

(71) Applicants: UNIVERSITY OF FLORIDA RESEARCH FOUNDATION, INCORPORATED, Gainesville, FL (US); Board of Regents, The University of Texas System, Austin, TX (US)

(72) Inventors: Elias Sayour, Newberry, FL (US); Adam J. Grippin, Houston, TX (US); Steven Hsesheng Lin, Houston, TX (US); Hector Ruben Mendez-Gomez, Gainesville, FL (US); Christiano Marconi, Gainesville, FL (US); Dhruvkumar Miteshkumar Soni, Gainesville, FL (US); Sage Alexander Copling, Houston, TX (US)

(21) Appl. No.: 19/382,913

(22) Filed: Nov. 7, 2025

Related U.S. Application Data
(60) Provisional application No. 63/717,622, filed on Nov. 7, 2024.
(60) Provisional application No. 63/849,674, filed on Jul. 23, 2025.

(51) Int. Cl. A61K 39/215; A61K 9/51; A61K 31/7088; A61K 39/00; A61K 39/395; A61P 35/00

(52) U.S. Cl. CPC A61K 39/215; A61K 9/5123; A61K 31/7088; A61K 39/39558; A61P 35/00; A61K 2039/505; A61K 2039/53; A61K 2039/55555; A61K 2039/585

(57) ABSTRACT

Disclosed are methods of increasing sensitivity of a tumor to treatment with an immune checkpoint inhibitor (ICI). The methods comprise administering to a subject in need thereof a composition comprising a lipid nanoparticle comprising mRNA encoding SARS-CoV-2 Spike protein, and administering an ICI to the subject, where the composition comprising a lipid nanoparticle comprising mRNA encoding SARS-CoV-2 Spike protein is optionally administered within 100 days of administration of the ICI, including with 30 days of administration of the ICI. In some aspects, the methods comprising administering to a subject in need thereof a composition comprising a lipid nanoparticle comprising mRNA encoding non-tumor antigens, and administering an ICI to the subject where the composition comprising a lipid nanoparticle comprising mRNA encoding non-tumor antigens is optionally administered within 100 days of administration of the ICI, including with 30 days of administration of the ICI.

### CROSS REFERENCE TO RELATED APPLICATIONS

This application claims the benefit of priority of U.S. Provisional Patent Application No. 63/717,622, filed on Nov. 7, 2024, and U.S. Provisional Patent Application No. 63/849,674, filed on Jul. 23, 2025, the disclosures of which are hereby incorporated by reference in their entireties.

### GRANT FUNDING DISCLOSURE

This invention was made with government support under Grant Numbers R37 CA251978, R01 CA266857, T32CA196561, P50CA221703, R01 FD007268, and CA016672, awarded by the National Institutes of Health. The government has certain rights in the invention.

### FIELD OF THE INVENTION

This application relates to use of lipid nanoparticles comprising nucleic acid molecules to enhance efficacy of immune checkpoint inhibitors in the treatment of cancer.

### BACKGROUND

While immune checkpoint inhibitors (ICI) have substantially extended survival in a subset of patients, the majority fail to respond to these treatments. These poor responses are attributed to immunosuppressive tumor microenvironments (TMEs) characterized by tolerogenic dendritic cells (DCs), myeloid suppressor cells and regulatory T cells. There are currently no clinically available methods to improve responses to ICI by modifying the TME. COVID mRNA vaccines induce robust stimulation of cytokine secretion. However, the impact of COVID mRNA vaccines on immune therapy is unknown.

### SUMMARY

Personalized mRNA vaccines sensitize tumors to ICIs in part by unleashing a cytokine/chemokine cascade that broadly activates immune cells. Since this cascade is not dependent on mRNA species, mRNA vaccines encoding non-tumor-specific antigens might also be used to reset immunotolerance and sensitize response to ICIs. The disclosure demonstrates that mRNA vaccines that do not encode tumor antigen (e.g., targeting SARS-CoV-2 spike protein) dramatically augment responses to ICIs. To illustrate, in two cohorts of patients with non-small cell lung cancer and metastatic melanoma, receipt of a COVID mRNA vaccine within 100 days of ICI initiation was associated with near doubling of overall survival. Preclinical models confirmed that mRNA vaccines targeting SARS-CoV-2 sensitize response to ICIs. By inducing a surge in interferon-α and Th1 chemokines, spike mRNA vaccines mediate antigen presenting cell co-localization with T cells in lymphoid organs for induction of anti-tumor immunity. Due to increases in PD-L1 on tumor cells, concomitant ICI treatment elicits increased PD-1+ T cells and epitope spreading against cancer associated antigens. Similar correlates of response were seen in both patients and healthy volunteers. Together, these results demonstrate that clinically available mRNA vaccines targeting non-tumor antigens are potent immune modulators capable of sensitizing tumors to ICIs.

Various aspects of the disclosure are summarized below:
1. A method of increasing sensitivity of a tumor to treatment with an immune checkpoint inhibitor (ICI), the method comprising (a) administering to a subject in need thereof a composition comprising a lipid nanoparticle comprising mRNA and (b) administering an ICI to the subject.
2. A method of treating a subject with an immune checkpoint inhibitor (ICI)-resistant tumor, the method comprising (a) administering to the subject in need thereof a composition comprising a lipid nanoparticle comprising mRNA and (b) administering an ICI to the subject.
3. The method of aspect 1 or 2, wherein the ICI is a PD-L1 inhibitor, a PD-1 inhibitor, or a CTLA4 inhibitor, such as an anti-PD-L1 antibody, anti-PD-1 antibody, or anti-CTLA4 antibody.
4. The method of any one of aspects 1-3, wherein the mRNA does not encode a tumor antigen, such as a tumor antigen derived from the subject.
5. The method of aspect 4, wherein the mRNA encodes a viral antigen, such as SARS-CoV-2 Spike protein.
6. The method of any one of aspects 1-5, wherein the composition comprising a lipid nanoparticle comprising mRNA is administered to the subject within 100 days (e.g., 51-100 days or 100 days) before or after the ICI is administered to the subject, optionally within 100 days after ICI administration, optionally within 51-100 days after administering the ICI to the subject.
7. The method of aspect 6, wherein the composition comprising a lipid nanoparticle comprising mRNA is administered to the subject within 30 days before or after the ICI is administered to the subject.
8. The method of any one of aspects 1-7, wherein the subject is suffering from non-small cell lung cancer or melanoma.
9. The method of any one of aspects 1-8, wherein the lipid nanoparticle comprises mRNA, SM-102, polyethylene glycol [PEG] 2000 dimyristoyl glycerol [DMG], cholesterol, and 1,2-distearoyl-sn-glycero-3-phosphocholine [DSPC].
10. The method of any one of aspects 1-8, wherein the lipid nanoparticle comprises mRNA, ((4-hydroxybutyl)azanediyl)bis(hexane-6,1-diyl)bis(2-hexyldecanoate), 2-(polyethylene glycol 2000)-N,N-ditetradecylacetamide, 1,2-distearoyl-sn-glycero-3-phosphocholine, and cholesterol.
11. The method of any one of aspects 1-10, wherein the composition comprising a lipid nanoparticle comprising mRNA is administered to the subject on the ipsilateral side relative to the tumor.

### BRIEF DESCRIPTION OF THE DRAWINGS

FIGS. 1A-1E: COVID mRNA vaccines are associated with improved survival in patients with NSCLC and metastatic melanoma. Survival for patients with NSCLC (FIG. 1A-FIG. 1C) or metastatic melanoma (FIG. 1D, FIG. 1E) treated with ICI who received a COVID mRNA vaccine within 100 days of initiating ICI or did not receive a COVID mRNA vaccine. Overall survival is shown for all NSCLC patients (FIG. 1A), those with Stage III disease only (FIG. 1B), and those with Stage IV disease only (FIG. 1C). Adjusted hazard ratios were calculated by Cox Proportional Hazards Regression (Tables 4, 7, and 8) including all variables that were significantly associated with survival on univariate analysis (Tables 3, 5, and 6). The number of patients at risk at each timepoint are indicated underneath each graph. Numbers comparing treatments are p values calculated by Gehan-Breslow-Wilcoxon Tests.

FIGS. 1F-1I: COVID mRNA Vaccines are associated with improved survival patients with NSCLC who receive ICI. FIGS. 1F-1H, Overall survival for NSCLC patients who received immune therapy and obtained a COVID mRNA vaccine differentiated by vaccine manufacturer (FIG. 1F), whether the patient received their first vaccine during this period (“Prime only”), a booster (“Boost only”), or both a priming vaccine and a booster vaccine within the 100 day period (“Prime and Boost”) (FIG. 1G), and number of vaccines received within 100 days of ICI initiation (FIG. 1H). FIG. 1I, Overall survival for NSCLC patients who did not receive treatment with immune therapy. Groups differentiate patients who did or did not receive a COVID mRNA vaccine within 100 days of biopsy.

FIGS. 2A-2O: Spike RNA-LNP prime anti-cancer immunity in an IFN-I dependent manner. FIG. 2A depicts a graphical experimental design. FIGS. 2B-2C depict tumor volume measurements of animals inoculated with B16F0 and vaccinated with spike RNA-LNP i.m. (Day 3, 6, 17) with and without ICI (PD-L1 mAbs) and IFNAR1 or IL1R mAbs. FIG. 2D depicts a schema for experiment. FIG. 2E depicts tumor volume measurements of animals inoculated with B16F0 and vaccinated with spike versus pp65 RNA-LNP i.m. (Day 3, 6) with and without ICI (PD-L1 mAbs) and IFNAR1 mAbs. FIGS. 2F-2G, IFN-α plasma ELISA (FIG. 2F) and IP-10 (FIG. 2G) from subcutaneous B16F0 bearing C57Bl/6 animals (n=8/group) 24 hours after one spike RNA-LNP vaccine i.m. FIGS. 2H-2J, Box-Plots of cellular phenotyping within 24 h of 3rd spike RNA-LNP vaccine i.m. (Days 3, 6, 20) from spleens of subcutaneous B16F0 bearing C57Bl/6 animals (n=5/group) for percentage of activated dendritic cells (FIG. 2H), macrophages (FIG. 2I), and MHC class II expressing monocytes (iMos) (FIG. 2J). FIG. 2K-FIG. 2M, PD-L1 median fluorescent intensity (MFI) on activated mature dendritic cells (FIG. 2K), macrophages (FIG. 2I) and MHCII-expressing inflammatory monocytes (FIG. 2M). FIGS. 2N-2O, Prevalence of CD86+Ly6C+ monocytes (FIG. 2N) and PDL1-expression myeloid cells (FIG. 2O) in tumors. Whiskers extend to highest and lowest values from a box drawn between 1st and 3rd quartiles with a line centered at median. Significance was determined by two-way ANOVA/mixed-effect analysis (FIG. 2B, FIG. 2E) and unpaired t tests (FIGS. 2F-2O).

FIGS. 2P-2X: Synthesis and characterization of Spike mRNA vaccines approximating BNT162b2. FIG. 2P, structure of LNPs and sequence map of mRNA. FIG. 2Q, Quality of mRNA assessed on a BioAnalyzer. FIG. 2R, Total anti-Spike IgG generated from multiple doses of vaccine. FIG. 2S, Visualization of mRNA loading in LNPs via gel electrophoresis. FIG. 2T, Size distribution of LNPs assessed by DLS. FIG. 2U, Size distribution determined via nanoparticle tracking analysis. FIG. 2V, Table of LNP properties. FIG. 2W, Surface zeta potential of mRNA-LNPs vs Empty LNPs. FIG. 2X, pH and zeta potential at biologically relevant levels of sodium bicarbonate.

FIGS. 3A-3L: Spike RNA-LNPs generate tumor-reactive T cells that increase PD-L1 expression on tumor cells. FIGS. 3A-3B, Frequency of activated effector (a) and central memory (b) T cells in spleens of tumor bearing mice 24 hours after three mRNA vaccines. FIG. 3C-FIG. 3D, Box-Plots of cellular phenotyping within 24 h of 3rd spike RNA-LNP vaccine i.m. (Days 3, 6, 20) from spleens of subcutaneous B16F0 bearing C57Bl/6 animals (n=5/group) for PD-1 expression in total CD44+ T cell compartment (c) and median fluorescence intensity (MFI) of PD-1 on effector CD8 T cells (d). Whiskers extend to highest and lowest values quartile from box drawn between 1st and 3rd quartiles with line centered at median. FIG. 3E-FIG. 3F, Representative AIM assay flow plots (e) and percent of antigen recognizing cells normalized to media only control (f). FIG. 3G, Fold change in tumor PD-L1 expression from mean of UT/ICI groups versus designated treatments in CD45 negative tumor cells by flow cytometry. FIG. 3H-FIG. 3I, Blinded manual counting of PD-L1+ tumor cells (SOX10) by IHC within 24 h of 3rd spike RNA-LNP vaccine i.m. (Days 3, 6, 20) from subcutaneous B16F0 bearing C57Bl/6 animals. For IHC samples, “Control” group is merger of controls (UT, ICI) into a single group versus RNA-LNP which represents merger of RNA-LNP and merged with RNA-LNP+ICI, percent of PD-1 expressing CD3+ TILs. FIGS. 3J-3L, Blinded manual counting of PD-1+ CD3+ by IHC within 24 h of 3rd spike RNA-LNP vaccine i.m. (Days 3, 6, 20) from subcutaneous B16F0 bearing C57Bl/6 animals. Fold change in CD3+ cells determined from baseline mean expression of UT/ICI groups. Significance determined by unpaired t test (FIGS. 3A-3D, FIGS. 3G-3H), two-way ANOVA/mixed-effect analysis (FIG. 3F), and Mann-Whitney test (FIGS. 3J-3K).

FIGS. 3M-3N: Sensitivity to ICI is improved by booster administration. FIG. 3M, Tumor response to vaccine regimen with two boosters. FIG. 3N, Tumor response to vaccine regimen with a single booster.

FIGS. 4A-4I: COVID mRNA vaccines generate a surge in IFN-alpha, innate immune activation and adaptive immunity in humans. FIG. 4A, Schematic depicting the experimental design wherein blood was drawn from five healthy subjects at baseline and 6 hours, 24 hours, 7 days, and 14 days after COVID mRNA immunization. FIG. 4B, Volcano plot of differentially expressed cytokines 24 hours after immunization with a COVID mRNA vaccine in five healthy subjects. Significantly elevated cytokines after corrections for multiple comparisons tests are labeled and colored red. FIG. 4C, Heat map displaying dynamic expression of the cytokines that are significantly elevated at 24 hours at 6 hours, 24 hours, 7 days, and 14 days after COVID mRNA vaccination. The only cytokine that is statistically different from baseline at other timepoints is IL-6, which is significantly elevated compared to baseline at 6 hours. FIG. 4D, individual data points highlighting changes in expression of IFN-α from baseline to 24 hours for each of five healthy volunteers. FIGS. 4E-4I, PD-L1 expression on circulating myeloid cells (FIG. 4E) and dendritic cells (FIG. 4F), activation of circulating dendritic cells quantified as percentages of CD80+CD86+ of CD11c+ cells (FIG. 4G), activation of NK cells (FIG. 4H), and activation of T cells expressed at numbers of CD69+ CD8+ cells (FIG. 4I) at 6 hours (6 h), 24 hours (24 h), 7 days (7 d), and 14 days (14 d) after immunization. Bars represent standard error. p values in FIGS. 4E-4I are results of paired t tests.

FIGS. 4J-4U: RNA-LNPs cause a dramatic shift in systemic cytokines/chemokines. FIGS. 4J-4U, Cytokine/chemokine multiplex panel. For samples within the standard curve, values are represented as pg/mL (FIGS. 4J-4S) and as fluorescent intensity values for cytokines above the standard curve (FIGS. 4T-4U). Plasma from subcutaneous B16F0 bearing C57Bl/6 animals (n=8/group) 24 hours after one spike RNA-LNP vaccine i.m. Significance was determined by and unpaired t tests.

FIGS. 5A-5H: COVID mRNA vaccines are associated with increased PD-L1 expression on tumors across a broad range of histologies. FIG. 5A, Schematic communicating that patients with NSCLC with biopsies documenting tumor proportion score (TPS) of PD-L1 (n=2,358) were separated into groups based on the timing of their most recent COVID mRNA vaccine prior to biopsy. FIG. 5B, TPS of PD-L1 for patients who received no vaccine prior to biopsy (“No Vaccine”), a vaccine within 100 days prior to biopsy (“<100 Days”), or a vaccine greater than 100 days prior to biopsy (“>100 Days”). FIG. 5C, Violin plot displaying the distribution of TPS values for each group. Numbers indicate the percentages of biopsy samples with PD-L1 expression >=50%. Numbers above graph are p values calculated by chi-square test comparing numbers of samples with TPS >=50% or <50%. FIG. 5D, TPS for patients who received influenza (left) and pneumonia (right) vaccines stratified by the interval from their most recent vaccine. ns=not significant by unpaired t test. FIG. 5E, Schematic representation of a search query for all patients with a report of “PD-L1” expression on tumor biopsy (either TPS or CPS) between August 2019 and November 2023. FIG. 5F, Pie charts describing the many histologies and primary tumor locations included in this diverse patient population. FIG. 5G, PD-L1 expression in tumors from the “Tissue agnostic cohort” quantified as either TPS (left) or CPS (right) and stratified by the interval from the patients' most recent COVID mRNA immunization. FIG. 5H, TPS and CPS stratified by the interval between biopsy and the patients' most recent influenza vaccines. P values are calculated by unpaired t test unless otherwise noted. Bar graphs display means and error bars represent standard error.

FIG. 5I: Gating strategy for murine splenic myeloid panel.

FIG. 6: Gating strategy for AIM Assay.

FIGS. 7A-7B: Representative images of PD-L1 expression in each treatment group. FIG. 7A, Keyence imaging of PD-L1 expression from whole tumor slides harvested from animals after vaccine 3. FIG. 7B, Confocal images of representative slices used to quantify PDL1 expression (Green) on SOX10+ (Red) tumor cells in each group. Nuclei stained with DAPI appear blue.

FIG. 8: Representative images of PD-L1 expression in each treatment group. Confocal images of representative slices used to quantify PD-1 expression (Green) on CD3+ (Red) tumor cells in each group. Nuclei stained with DAPI appear blue.

FIGS. 9A-9D: Cytokine production in healthy subjects after SARS-COV-2 mRNA immunization.

FIG. 10: depicts a schematic illustrating potential mechanism by which COVID mRNA vaccines augment responses to immune checkpoint blockade.

FIGS. 11A-11F depict Kaplan-Meier survival plots of the overall survival of patients with non-small cell lung cancer (NSCLC) who received COVID-19 mRNA vaccinations (either the Moderna or the Pfizer COVID-19 vaccine) and immune checkpoint inhibitors (ICI) or systemic therapies.

FIGS. 12A-12F depict six Kaplan-Meier survival plots assessing the overall survival of NSCLC (non-small cell lung cancer) patients receiving immune checkpoint inhibitors (ICIs), stratified by vaccine status. The analysis includes the impact of COVID mRNA vaccines, pneumonia vaccines, and influenza vaccines, with adjustments for immortal time bias.

FIGS. 13A-13B depict Kaplan-Meier survival plots of overall survival data for patients with non-small cell lung cancer (NSCLC) undergoing immune checkpoint inhibitor (ICI) therapy, comparing those who received a COVID mRNA vaccine within 100 days of ICI initiation to those who did not receive the vaccine. Two patient groups are analyzed, patients with unresectable Stage III NSCLC (FIG. 13A) and patients with Stage IV NSCLC (FIG. 13B).

FIGS. 14A-14B depict graphs of overall survival outcomes for patients with Stage III non-small cell lung cancer (NSCLC) or resectable Stage III NSCLC treated with immune checkpoint inhibitors (ICI), stratified based on whether they received a COVID mRNA vaccine within 100 days of initiating ICI therapy.

FIGS. 15A-15B depict graphs comparing the survival outcomes for Stage IV melanoma patients treated with immune checkpoint inhibitors (ICI), with or without receiving a COVID mRNA vaccine within 100 days of initiating ICI.

FIGS. 16A-16D depict graphs of the overall survival (OS) in Stage IV melanoma patients undergoing immune checkpoint inhibitor (ICI) therapy, stratified by different variables related to COVID-19 mRNA vaccination including Moderna mRNA vaccine or Pfizer mRNA vaccine, prime dose, boost dose, one or two vaccines, or COVID mRNA vaccine prior to ICI start.

FIGS. 17A-17B depict graphs of the overall survival outcomes for patients with NSCLC (non-small cell lung cancer, FIG. 17A) and Stage IV melanoma (FIG. 17B) undergoing immune checkpoint inhibitor (ICI) therapy, receiving a COVID vaccine during the pandemic era compared to patients not receiving a COVID vaccine.

FIG. 17C depicts a table of a multivariate Cox regression analysis of variables that may impact survival for both cohorts.

FIGS. 18A-18C depict graphs that illustrate the overall survival curves for melanoma patients treated with immune checkpoint inhibitors (ICI), stratified based on whether they received a COVID mRNA vaccine within 100 days of initiating ICI. FIG. 18A depicts a graph of all melanoma patients treated with ICI, FIG. 18B depicts a graph of Stage III melanoma patients treated with ICI, and FIG. 18C depicts a graph of Stage IV melanoma patients treated with ICI.

FIG. 19A depicts a schematic of the timeline of administration of PD-L1 mAb and a COVID vaccine using an RNA lipid nanoparticle in a mouse model of B16F0 melanoma.

FIG. 19B depicts a graph of the tumor size for B16F0 mice administered immune checkpoint inhibitor (ICI), RNA-LNP, RNA-LNP+ICI, or untreated.

FIG. 19C depicts a schematic of the experimental design of administration of PD-L1 mAb and a COVID vaccine using an RNA lipid nanoparticle to a mouse model of Lewis Lung Carcinoma (LLC).

FIG. 19D depicts a graph of the number of lung metastases in LLC mice untreated or treated with ICI alone, RNA-nanoparticle alone, or the combination of ICI+RNA-nanoparticle.

FIG. 19E depicts representative images of metastatic tumor burden in control, ICI treated, RNA-LP treated or RNA-LNP+ICI treated lungs from LLC mice.

FIG. 19F depicts a schematic of the experimental design using mice with orthotopic LLC tumors receiving RNA-LNPs with or without anti-PD-L1 mAbs.

FIG. 19G depicts a graph of lung weight as an indication of tumor burden in control, ICI treated, RNA-LP treated, or RNA-LNP+ICI treated mice with orthotopic LLC tumors.

FIG. 19H depicts representative images of metastatic tumor burden in control, ICI treated, RNA-LP treated, or RNA-LNP+ICI treated lungs from mice with orthotopic LLC tumors.

FIG. 20A depicts an experimental schematic using mice with subcutaneous B16F0 tumors treated with Spike RNA-LNPs or exogenous interferon alpha injections.

FIG. 20B depicts a graph of tumor growth comparing Spike RNA-LNP+PD-L1 antibody treatment to interferon alpha+PD-L1 antibody in mice with subcutaneous B16F0 tumors.

FIG. 20C depicts an experimental schematic using mice with subcutaneous B16F0 tumors treated with PD-1 blocking antibodies (aPD1) alone or combined with RNA-LNPs or Poly I:C to assess tumor growth reduction.

FIG. 20D depicts a graph of tumor growth for mice with subcutaneous B16F0 tumors comparing Spike RNA-LNP+PD-L1 antibody treatment to Poly I:C+PD-L1 antibody treatment or PD-L1 antibody treatment alone.

FIG. 20E depicts an experimental schematic using mice with subcutaneous B16F0 tumors treated with anti-PD-L1 and RNA-LNPs containing silenced (N1-methyl pseudouridine modified) or unsilenced mRNA coding for Spike protein or antigen pp65.

FIG. 20F depicts a graph of tumor growth for mice with subcutaneous B16F0 tumors comparing anti-PD-L1 and RNA-LNPs containing either silenced (N1-methyl pseudouridine modified) or unsilenced mRNA coding for either Spike protein or antigen pp65.

FIG. 20G depicts boxplot graphs of tumor volumes at days 17 and 20 in mice with mice with subcutaneous B16F0 tumors, illustrating the statistical differences between various treatments

FIG. 21A summarizes quality control analysis of dsRNA contamination in RNA samples using background subtraction in lipid nanoparticles with purified spike RNA and single-stranded RNA.

FIG. 21B depicts a schematic of the experimental design and a graph of in vivo tumor growth data for B16F0 tumor-bearing mice treated with anti-PD-L1 monoclonal antibodies (ICI) given alone or in combination with Spike RNA-LNPs (RNA-LNP+ICI), ssRNA-LNPs (ssRNA-LNP+ICI), or anionic lipoplexes (anionic LPX+ICI).

FIG. 21C depicts a graph of the results of an ELISA assay showing serum IFN-α levels in wildtype mice or RIG-1−/− mice treated with RNA-LNP.

FIG. 21D depicts representative images of secondary structure analysis of RNA (non-complexed RNA and RNA extracted from LNPs) using a tape station under different heating conditions.

FIGS. 22A-22M depict graphs of a systemic cytokine/chemokine multiplex panel in plasma from subcutaneous B16F0 bearing C57Bl/6 animals (n=8/group) 24 hours after administration of varying formulations including ICI, empty RNA-lipid nanoparticles (RNA-LNPs), Spike RNA-LNP, ICI+RNA-LNP, and RNA-LNP+ICI+alFNAR1. Significance was determined by unpaired t tests. Values are represented as pg/mL

FIG. 23 depicts representative flow cytometry plots for gating strategy for an AIM Assay.

FIG. 24A depicts a schematic of an experimental timeline and treatment groups examining PD-L1 expression on tumor cells in relation to Type I interferon (IFN) signaling in a B16F0 melanoma mouse model.

FIG. 24B depicts a graph of PD-L1 expression on CD45 tumor cells across different treatment conditions including untreated mice, mice treated with IFNAR1-blocking antibodies (IFNAR1 mAb), mRNA vaccines alone (RNA-LNP), combined treatments (mAb+RNA-LNP), and both wild-type and IFN-gamma KO mice.

FIGS. 25A-25B depict volcano plots of the cytokine production in healthy individuals 24 hours post-vaccination with a COVID-19 mRNA vaccine (Moderna in FIG. 25A; Pfizer in FIG. 25B).

FIG. 26 depicts representative flow cytometry plots of the gating strategy for analyzing patient samples to identify specific immune cell subsets within a population of cells.

FIG. 27A depicts a schematic of the experimental design where blood samples were collected from healthy subjects at five timepoints post-vaccination with COVID-19 mRNA (BNT162b2).

FIG. 27B depicts a heatmap of the dynamic changes in cytokine levels in the blood samples were collected from healthy subjects at five timepoints post-vaccination with a COVID-19 mRNA vaccine.

FIGS. 27C-27D depict graphs of the fold change and absolute concentration (pg/mL) of interferon alpha (IFN-α) at baseline and 24 hours post-vaccination with a COVID-19 mRNA vaccine.

FIGS. 27E-27F depict graphs of PD-L1 expression on circulating myeloid cells (FIG. 27E) and dendritic cells (FIG. 27F) at time points up to 48 hours post vaccination.

FIG. 27G depicts a graph of the increase in NK cell activation at time points up to 48 hours post vaccination.

FIG. 27H depicts a graph of the activation of T cells (marked by CD69+, CD8+ expression) at time points up to 48 hours post vaccination.

FIG. 28 depicts a heatmap of the differential expression levels of cytokines at two time points, 6 hours and 24 hours, for patients receiving two different mRNA-based COVID vaccine formulations: Spikevax (2023-2024 formulation) and Comirnaty (2024-2025 formulation). The cytokines shown include IFNA1/IFNA13, IFNW1, CXCL10, IL1RN, CSF3, CCL8, and IL6. The color scale indicates log 2 fold changes, with blue representing downregulation and yellow-red indicating upregulation.

FIGS. 29A-29B depict graphs of the moving average of PD-L1 expression over time following COVID mRNA vaccine immunization for NSCLC patients (FIG. 29A) or Tissue-agnostic patients (FIG. 29B).

FIGS. 30A-30D depict graphs of the overall survival of patients with metastatic NSCLC receiving a COVID mRNA vaccine within 100 days of initiating ICI and having baseline PD-L1 expression at baseline biopsy TPS<1% (FIG. 30A), TPS of between 1-49.9% (FIG. 30B), or a TPS of ≥50% (FIG. 30C). FIG. 30D depicts a graph of the overall survival of patients who did not receive a COVID vaccine during the pandemic era (e.g., after Sep. 2, 2020) versus patients in the pre-pandemic era (before Sep. 2, 2020).

FIGS. 31A-31C depict graphs of the survival for patients with NSCLC treated with ICI who received a COVID-19 mRNA vaccine within 100 days of initiating ICI or did not receive a COVID-19 mRNA vaccine. FIG. 31A depicts a graph of all patients with NSCLC. FIG. 31B depicts a graph of patients with unresectable stage III NSCLC. FIG. 31C depicts a graph of patients with stage IV NSCLC.

FIGS. 31D-31E depicts graph of the survival for patients with metastatic melanoma treated with ICI who received a COVID-19 mRNA vaccine within 100 days of initiating ICI or did not receive a COVID-19 mRNA vaccine. For FIGS. 31A-31E, p values and HR_{adj} were calculated using two-sided Cox proportional hazards regression including all variables that were significantly associated with survival on univariable analysis. The number of patients at risk at each timepoint is indicated below each graph.

FIG. 32A depicts the experimental design and tumor volume of mice inoculated with B16F0 cells. Groups included untreated (UT; n=7; circles), anti-PD-1 (n=8; squares), RNA-LNPs (n=8; triangles) and RNA-LNPs+anti-PD-1 (n=8; inverted triangles). mAb, monoclonal antibody.

FIG. 32B depicts a graph of the experimental design and tumor volume for mice inoculated with LLC cells. Groups included untreated (n=8; circles), anti-PD-1 (n=9; squares), RNA-LNP (n=9; triangles) and RNA-LNPs+anti-PD-1 (n=9; inverted triangles).

FIG. 32C depicts a graph of the experimental design and tumor volume measurements for mice inoculated with LLC cells. Groups included untreated (n=9; circles), anti-PD-1 (n=10; squares), RNA-LNP (n=7; triangles) and RNA-LNPs+PD-1 (n=8; inverted triangles).

FIG. 32D depicts a graph of the experimental design and tumor volume for mice inoculated with B16F0 cells. n=12 per group.

FIG. 32E depicts a graph of IFNα plasma enzyme linked immunosorbent assay (ELISA) from B16F0-tumour-bearing mice (n=8 per group) 24 h after one RNA-LNP vaccine (day 3).

FIG. 32F depicts a schematic of the timeline of administration of RNA-LNP and PDL1 mAb to mice for FIGS. 32G-32H.

FIGS. 32G-32H depict graphs of cellular phenotyping within 24 h of vaccine 3 (days 3, 6 and 20) of cells from spleens of mice bearing B16F0 tumors (n=5 per group), including the percentage of activated (CD80+CD86+) DCs (FIG. 32G) and macrophages (FIG. 32H).

FIGS. 32I-32J depict graphs of PD-L1 median fluorescence intensity (MFI) on activated mature DCs (FIG. 32I) and macrophages (FIG. 32J).

FIGS. 32K-32P depict graphs of the characterization of antigen presentation among myeloid cells in tumor draining lymph nodes (tdLNs) and spleens of mice bearing B16F10-ova tumors 24 h after vaccine 2 (days 10 and 13). FIG. 32K depicts a graph of the overall percentage of CD45+ cells that express MHC-II⁺. FIG. 32L depicts a graph of the percentage of MHC-II⁺ cells presenting SIINFEKL. FIG. 32M depicts a graph of the percentage of SIINFEKL-presenting MHCII⁺ cells that express the activation marker CD86. FIG. 32N depicts a graph of the percentage of CD45⁺ cells that are MHC-II⁺Ly6C⁺. FIG. 32O depicts a graph of SIINFEKL⁺MHC-II⁺Ly6C⁺ cells as a percentage of all CD45⁺ cells. FIG. 32P depicts a graph of the Ly6C⁺MHCII⁺ cells as a percentage of total SIINFEKL-presenting cells. Significance was determined using two-way analysis of variance (ANOVA)/mixed-effect analysis with Geisser-Greenhouse correction (FIGS. 32A-32D) and two-tailed unpaired t-tests (FIGS. 32E-32P). n indicates the number of biologically independent samples. For the box plots, the whiskers extend to the highest and lowest values, the box limits show the first and third quartiles and the center line shows the median value. For FIGS. 32A-32D, data are mean±s.e.m.

FIGS. 33A-33B depict graphs of the percentage of activated effector (FIG. 33A) and effector memory (FIG. 33B) T cells in the spleens of tumor-bearing mice on day 21 (vaccine days 3, 6, 17) (n=5 per group). Significance was determined using two-tailed unpaired t-tests.

FIG. 33C depicts a graph of the percentage of tetramert cells of splenic CD8⁺ T cells collected from mice bearing B16F0 tumors on day 21 (vaccination days 14 and 17). Groups include untreated (n=6), anti-PD-1 (n=6), RNA-LNPs (n=5) and RNA-LNPs+anti-PD-1 (n=7). Significance was determined using two-tailed unpaired t-tests.

FIG. 33D depicts the normalized percentage of AIM⁺ T cells after splenocyte co-culture with overlapping peptide pools. Significance was determined using two-tailed Brown-Forsythe and Welch ANOVA, followed by Dunnett's T3 multiple-comparison test

FIG. 33E depicts representative images of PD-1⁺CD3⁺ cells by immunofluorescence 24 h after vaccine 3 (days 3, 6 and 20) from s.c. tumors of B16F0-tumour-bearing mice treated with or without anti-PD-L1. For FIG. 33E, scale bars, 100 μm. AF647, Alexa Fluor 647.

FIG. 33F depicts a graph of blinded manual counting (n=4 tumors per group with 4 counts per tumor) of the images from FIG. 33E. Significance was determined using two-tailed Welch's t-test.

FIG. 33G depicts a graph of the percentage PD-1⁺CD8⁺ cells of CD3⁺ T cells in tumors of B16F0-bearing mice vaccinated with RNA-LNPs (days 14 and 17). Groups included untreated (n=7), anti-PD-L1 (n=8), RNA-LNPs (n=9) and RNA-LNPs+anti-PD-L1 (n=7).

FIG. 33H depicts a graph of the pooled tetramer positivity (%) among CD8⁺ T cells in B16F0 tumors. Groups included untreated (n=6), anti-PD-1 (n=6), RNA-LNPs (n=4) and RNA-LNPs+anti-PD-1 (n=7) (RNA-LNPs days 14 and 17).

FIG. 33I depicts a graph of PD-L1 expression on B16F0 tumor cells (CD45-FSC-Ahigh) isolated from mice 24 h after vaccine 3 (days 3, 6 and 17) as determined using flow cytometry. Groups included untreated (n=4) and RNA-LNPs (n=5).

FIG. 33J depicts a graph of blinded manual counting (n=6 tumors per group with 4 counts per tumor) of PD-L1⁺ tumor cells (SOX10) by immunofluorescence 24 h after vaccine 3 (days 3, 6, 21) from B16F0-tumour-bearing mice. The circle symbols indicate PBS treatment and the square symbols represent anti-PD-L1 treatment. The different shades represent individual tumors. Significance was determined using two-tailed unpaired t-tests. For the box plots, the whiskers extend to the highest and lowest values, the box limits show the first and third quartiles and the center line shows the median value. n values indicate biologically independent samples unless indicated otherwise.

FIG. 33K depicts representative images of PD-L1⁺ tumor cells (SOX10) by immunofluorescence 24 h after vaccine 3 (days 3, 6, 21) from B16F0-tumor-bearing mice. For FIG. 33K, scale bars, 50 μm.

FIG. 34A depicts a schematic of the experimental design in which blood was drawn from five healthy individuals at baseline and 6 h, 24 h, 7 days and 14 days after Spikevax (mRNA-1273) COVID-19 mRNA immunization.

FIG. 34B depicts a heat map of the dynamic expression of the cytokines that are significantly elevated at 24 h at 6 h, 24 h, 7 days and 14 days after COVID-19 mRNA vaccination. Significant variables were defined as those with P<0.05 and a log 2-transformed fold change with an absolute value of greater than 0.5 after linear modelling with fixed effects. Adjusted P values were calculated using moderated two-tailed t-tests with false-discovery rate (FDR) correction for multiple testing.

FIGS. 34C-34D depict graphs of Individual datapoints highlighting changes in expression of IFNα from baseline to 24 h for each of five healthy volunteers. Data are expressed as the fold change measured using the NULISAseq Inflammation Panel (FIG. 34C). The concentration was also measured separately with NULISAseq absolute quantification (AQ) (FIG. 34D).

FIGS. 34E-34F depict graphs of PD-L1 expression on circulating myeloid cells (CD3-CD19-CD56-CD11b+) (n=5) (FIG. 34E) and DCs (CD3-CD19-CD56-CD11c+MHC-II+) (n=5) (FIG. 34F) at 6 h, 24 h and 7 days after immunization.

FIGS. 34G-34H depict graphs of activation of natural killer cells (CD56⁺; n=5) (FIG. 34G), and T cells expressed as numbers of CD69⁺ cells of CD8⁺CD3⁺ cells (n=5) (FIG. 34H) at 6 h, 24 h, 7 days and 14 days after immunization. Data are mean±s.e.m. P values were calculated using two-tailed paired t-tests.

FIG. 35A depicts a schematic of patients with NSCLC biopsies documenting PD-L1 TPS.

FIG. 35B depicts a graph of TPS stratified by COVID-19 mRNA vaccination timing. P values were calculated using two-tailed unpaired t-tests.

FIG. 35C depicts a graph of the distribution of samples with TPS≥50%.

FIG. 35D depicts a graph of TPS stratified by influenza (left) or pneumonia (right) vaccination timing. P values were calculated using two-tailed unpaired t-tests

FIG. 35E depicts a schematic of biopsies documenting TPS or combined positive score (CPS) of PD-L1 (January 2020 to October 2023).

FIG. 35F depicts a pie chart of the primary tumor locations from the diverse cohort.

FIGS. 35G-35H depict graphs of TPS in a tissue-agnostic cohort stratified by COVID-19 mRNA immunization timing (FIG. 35G) or TPS stratified by timing of influenza vaccination (FIG. 35H). P values were calculated using two-tailed unpaired t-test for FIG. 35H and using a two-tailed unpaired t-test with Welch's correction for unequal variance for FIG. 35G.

FIG. 35I depicts a graph of the survival of patients in the tissue-agnostic cohort treated with ICI who received any COVID-19 vaccine within 100 days of initiating ICI or did not receive any COVID-19 vaccine. Top line in graph represents COVID vaccine within 100 days of ICI start.

FIG. 35J depicts a graph of the survival of patients in FIG. 35J stratified by receipt of COVID-19 vaccine before ICI. Top line in graph represents COVID vaccine within 100 days of ICI start.

FIG. 35K depicts a graph of the survival for patients in FIG. 35I who started ICI in the pandemic era (since Sep. 2, 2020, 100 days before mRNA vaccine approval). Survival analyses in the tissue-agnostic cohort were not limited to only those patients with a clear TPS value. Top line in graph represents patients receiving COVID vaccine.

FIGS. 36A-36D depict graphs of the overall survival (“OS”) for patients with metastatic stage IV NSCLC treated with ICI who received a COVID-19 mRNA vaccine within 100 days of initiating ICI or did not receive a COVID-19 vaccine who had baseline PD-L1 expression at baseline biopsy TPS<1% (FIG. 36A; top line in graph represents patients receiving COVID vaccine), 1-49.9% (FIG. 36B; top line in graph represents patients receiving COVID vaccine) or ≥50% (FIG. 36C; top line in graph represents patients receiving COVID vaccine). To evaluate the impact of vaccination in each clinical setting, patients were excluded if they received a COVID-19 mRNA vaccine before their biopsy. FIG. 36D depicts a graph of the OS of unvaccinated patients with stage IV NSCLC stratified by era of ICI start, who had baseline TPS<1% at biopsy. For FIGS. 36A-36D, P values and HRs were calculated using log-rank (Mantel-Cox, two-sided) tests.

FIG. 37A depict a graph of the OS of patients treated with ICI who did or did not receive a COVID mRNA vaccine within 100 days of initiating ICI. Top line in graph represents patients receiving COVID vaccine within 100 days of ICI.

FIG. 37B depicts a graph of the OS of patients treated with ICI who received a COVID mRNA vaccine within 100 days before initiating ICI or did not receive a COVID mRNA vaccination within that timeframe. Top line in graph represents patients receiving COVID vaccine within 100 days of ICI.

FIG. 37C depicts a graph of the OS of patients treated with an ICI who received COVID mRNA vaccination within 100 days of ICI, stratified by the time between closest mRNA vaccination and ICI start (30 days vs 30-100 days). Top line in graph represents patients receiving COVID vaccine within 30 days of ICI.

FIG. 37D depicts a graph of the OS of patients treated with ICI with no history of COVID mRNA vaccination who did or did not receive an influenza vaccine within 100 days of initiating ICI. For FIGS. 37A-37D HR, p values, and 95% CIs were calculated using log-rank methods.

FIG. 38A depicts a schematic of the experimental procedure of FIG. 38B.

FIG. 38B depicts a graph of interferon alpha concentration (“IFN-α”) in serum from wild-type, RIF RIG-I KO, or MDA-5 KO (MDA-5) mice 24 after treatment with RNA-LNPs. p values represent results of unpaired t tests.

FIG. 39A depicts a graph of tumor volume in mice with B16F0 tumors that were treated with ICI with or without RNA-LNPs or high molecular weight (HMW) poly I:C starting on Day 3. Poly I:C (no LNP) was administered intramuscularly.

FIG. 39B depicts a graph of tumor size in mice with B16F0 tumors that were treated with ICI with or without RNA-LNPs or low molecular weight (LMW) poly I:C starting on Day 14. Poly I:C (no LNP) was administered intramuscularly. Squares represent ICI treatment (anti-PD1), inverted triangles represent RNA-LNP+ICI treatment, and diamonds represent low molecular weight polyl:C+ICI

### DETAILED DESCRIPTION

The disclosure is based, at least in part, on the observation that the innate immune responses resulting from SARS-CoV-2 spike mRNA vaccination profoundly resets the cancer immunotherapy cycle and primes adaptive immunity for synergy with immune checkpoint inhibitors (ICIs). Receipt of a SARS-CoV-2 mRNA vaccine within, e.g., 100 days of ICI initiation was associated with substantial improvements in overall survival (OS) in patients with non-small cell lung cancer (NSCLC) and metastatic melanoma. In preclinical models, this effect was found to be dependent on a surge in type-1 interferon that drove activation and mobilization of antigen presenting cells (APCs) to lymphoid organs for co-localization with activated T cells. Although tumor cells subvert these primed responses by increasing PD-L1 expression, administration of ICIs sustains T cell responses and elicits epitope spreading against cancer associated antigens. Analogous response correlates were achieved in preclinical modeling for patients receiving COVID mRNA vaccines, including heightened IFN-α production, innate/adaptive immune activation and T cell-mediated increases in tumor PD-L1 expression. Together, the results described herein demonstrate that clinically available mRNA nanoparticle vaccines targeting non-tumor antigens are potent immune modulators capable of sensitizing tumors to ICI.

In various aspects, the disclosure provides a method of increasing sensitivity of a tumor to treatment with an immune checkpoint inhibitor (ICI), the method comprising (a) administering to a subject in need thereof a composition comprising a lipid nanoparticle comprising nucleic acid molecules (e.g., mRNA) and (b) administering an ICI to the subject. The disclosure also provides a method of treating a subject with a tumor, comprising (a) administering to the subject in need thereof a composition comprising a lipid nanoparticle comprising nucleic acid molecules (e.g., mRNA) and (b) administering an ICI to the subject. In this regard, the disclosure further provides a method of treating a subject with an immune checkpoint inhibitor (ICI)-resistant tumor, comprising (a) administering to the subject in need thereof a composition comprising a lipid nanoparticle comprising nucleic acid molecules (e.g., mRNA) and (b) administering an ICI to the subject.

The term “nanoparticle” refers to a particle that is less than about 1000 nm in diameter. As the nanoparticles of the present disclosure comprise lipids that have been processed to induce liposome formation, the presently disclosed nanoparticles in various aspects comprise liposomes. Liposomes are artificially-prepared vesicles which, in exemplary aspects, are primarily composed of lipid bilayer(s).

In exemplary aspects, the nanoparticle has a diameter within the nanometer range. In exemplary aspects, the nanoparticle has a diameter between about 50 nm to about 500 nm, e.g., about 50 nm to about 450 nm, about 50 nm to about 400 nm, about 50 nm to about 350 nm, about 50 nm to about 300 nm, about 50 nm to about 250 nm, about 50 nm to about 200 nm, about 50 nm to about 150 nm, about 50 nm to about 100 nm, about 100 nm to about 500 nm, about 150 nm to about 500 nm, about 200 nm to about 500 nm, about 250 nm to about 500 nm, about 300 nm to about 500 nm, about 350 nm to about 500 nm, or about 400 nm to about 500 nm. In exemplary aspects, the nanoparticle has a diameter between about 50 nm to about 300 nm, e.g., about 100 nm to about 250 nm, about 110 nm±5 nm, about 115 nm±5 nm, about 120 nm±5 nm, about 125 nm±5 nm, about 130 nm±5 nm, about 135 nm±5 nm, about 140 nm±5 nm, about 145 nm±5 nm, about 150 nm±5 nm, about 155 nm±5 nm, about 160 nm±5 nm, about 165 nm±5 nm, about 170 nm±5 nm, about 175 nm±5 nm, about 180 nm±5 nm, about 190 nm±5 nm, about 200 nm±5 nm, about 210 nm±5 nm, about 220 nm±5 nm, about 230 nm±5 nm, about 240 nm±5 nm, about 250 nm±5 nm, about 260 nm±5 nm, about 270 nm±5 nm, about 280 nm±5 nm, about 290 nm±5 nm, or about 300 nm±5 nm. In exemplary aspects, the nanoparticle is about 50 nm to about 250 nm in diameter. In some aspects, the nanoparticle is about 70 nm to about 200 nm in diameter. In various aspects, the nanoparticle has a diameter of about 200 nm to about 500 nm. In exemplary aspects, the nanoparticle is present in a pharmaceutical composition comprising a heterogeneous mixture of nanoparticles ranging in diameter, e.g., about 50 nm to about 500 nm or about 50 nm to about 250 nm in diameter or about 200 nm to about 500 nm in diameter.

In exemplary embodiments, the nanoparticles comprise a cationic lipid. In some embodiments, the cationic lipid is a low molecular weight cationic lipid such as those described in U.S. patent application No. 20130090372, the contents of which are herein incorporated by reference in their entirety. The cationic lipid in exemplary instances is a cationic fatty acid, a cationic glycerolipid, a cationic glycerophospholipid, a cationic sphingolipid, a cationic sterol lipid, a cationic prenol lipid, a cationic saccharolipid, or a cationic polyketide. In exemplary aspects, the cationic lipid comprises two fatty acyl chains, each chain of which is independently saturated or unsaturated. In some instances, the cationic lipid is a diglyceride. In exemplary instances, the cationic lipid is DOTAP (1,2-dioleoyl-3-trimethylammonium-propane), or a derivative thereof. In exemplary instances, the cationic lipid is DOTMA (1,2-di-O-octadecenyl-3-trimethylammonium propane), or a derivative thereof.

In some embodiments, the nanoparticles are formed from 1,2-dioleyloxy-N,N-dimethylaminopropane (DODMA) liposomes, DiLa2 liposomes from Marina Biotech (Bothell, Wash.), 1,2-dilinoleyloxy-3-dimethylaminopropane (DLin-DMA), 2,2-dilinoleyl-4-(2-dimethylaminoethyl)-[1,3]-dioxolane (DLin-KC2-DMA), and MC3 (U.S. Patent Application Publication No. 20100324120; herein incorporated by reference in its entirety). In some embodiments, the nanoparticles comprise liposomes formed from the synthesis of stabilized plasmid-lipid particles (SPLP) or stabilized nucleic acid lipid particle (SNALP) that have been shown to be suitable for oligonucleotide delivery in vitro and in vivo. The nanoparticles in some aspects are composed of 3 to 4 lipid components in addition to the nucleic acid molecules (mRNA). In exemplary aspects, the nanoparticle comprises 55% cholesterol, 20% disteroylphosphatidyl choline (DSPC), 10% PEG-S-DSG, and 15% 1,2-dioleyloxy-N,N-dimethylaminopropane (DODMA), as described by Jeffs et al., Pharm Res. 2005; 22 (3): 362-72. In exemplary instances, the nanoparticle comprises 48% cholesterol, 20% DSPC, 2% PEG-c-DMA, and 30% cationic lipid, where the cationic lipid can be 1,2-distearloxy-N,N-dimethylaminopropane (DSDMA), DODMA, DLin-DMA, or 1,2-dilinolenyloxy-3-dimethylaminopropane (DLenDMA), as described by Heyes et al., J. Control Release 2005; 107(2): 276-87.

In some aspects, the nanoparticles comprise a neutral lipid, such as cholesterol. For instance, the nanoparticles may comprise from about 25.0% cholesterol to about 40.0% cholesterol, from about 30.0% cholesterol to about 45.0% cholesterol, from about 35.0% cholesterol to about 50.0% cholesterol and/or from about 48.5% cholesterol to about 60% cholesterol. In some aspects, the nanoparticles do not comprise a neutral lipid, such as cholesterol.

In various instances, the cationic lipid comprises 2,2-dilinoleyl-4-dimethylaminoethyl-[1,3]-dioxolane (DLin-KC2-DMA), dilinoleyl-methyl-4-dimethylaminobutyrate (DLin-MC3-DMA), or di((Z)-non-2-en-1-yl) 9-((4-(dimethylamino) butanoyl)oxy) heptadecanedioate (L319), and the particle further comprises a neutral lipid, a sterol and a molecule capable of reducing particle aggregation, for example, a PEG or PEG-modified lipid.

The nanoparticle in various aspects comprises DLin-DMA, DLin-K-DMA, 98N12-5, C12-200, DLin-MC3-DMA, DLin-KC2-DMA, DODMA, PLGA, PEG, PEG-DMG, PEGylated lipids and/or amino alcohol lipids. In some aspects, the nanoparticle comprises a cationic lipid such as, but not limited to, DLin-DMA, DLin-D-DMA, DLin-MC3-DMA, DLin-KC2-DMA, DODMA and amino alcohol lipids. The amino alcohol cationic lipid comprises in some aspects lipids described in and/or made by the methods described in U.S. Patent Publication No. 20130150625, herein incorporated by reference in its entirety. As a non-limiting example, the cationic lipid in certain aspects is 2-amino-3-[(9Z,12Z)-octadeca-9,12-dien-1-yloxy]-2-{[(9Z,2Z)-octadeca-9,12-dien-1-yloxy]methyl}propan-1-ol (Compound 1 in U.S. Patent Publication No. 20130150625); 2-amino-3-[(9Z)-octadec-9-en-1-yloxy]-2-{[(9Z)-octadec-9-en-1-yloxy]methyl}propan-1-ol (Compound 2 in US Patent Publication No. 20130150625); 2-amino-3-[(9Z,12Z)-octadeca-9,12-dien-1-yloxy]-2-[(octyloxy)methyl]propan-1-ol (Compound 3 in US20130150625); 2-(dimethylamino)-3-[(9Z,12Z)-octadeca-9,12-dien-1-yloxy]-2-{[(9Z,12Z)-octadeca-9,12-dien-1-yloxy]methyl}propan-1-ol (Compound 4 in US Patent Publication No. 20130150625); or any pharmaceutically acceptable salt or stereoisomer thereof.

In various embodiments, the lipid nanoparticle comprises at least one lipid selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino) butanoyl)oxy) heptadecanedioate (L319). The lipid nanoparticle may comprise a neutral lipid, such as a neutral lipid selected from DSPC, DPPC, POPC, DOPE and SM. The lipid nanoparticle may comprise a sterol, e.g., cholesterol. The lipid nanoparticle may comprise a PEG-lipid, e.g., PEG-DMG or PEG-CDMA.

In exemplary aspects, the lipid may be selected from (20Z,23Z)—N,N-dimethylnonacosa-20,23-dien-10-amine, (17Z,20Z)—N,N-dimemylhexacosa-17,20-dien-9-amine, (1Z,19Z)—N,N-dimethylpentacosa-16, 19-dien-8-amine, (13Z,16Z)—N,N-dimethyldocosa-13, 16-dien-5-amine, (12Z,15Z)—N,N-dimethylhenicosa-12, 15-dien-4-amine, (14Z,17Z)—N,N-dimethyltricosa-14, 17-dien-6-amine, (15Z,18Z)—N,N-dimethyltetracosa-15, 18-dien-7-amine, (18Z,21Z)—N,N-dimethylheptacosa-18,21-dien-10-amine, (15Z,18Z)—N,N-dimethyltetracosa-15, 18-dien-5-amine, (14Z,17Z)—N,N-dimethyltricosa-14,17-dien-4-amine, (19Z,22Z)—N,N-dimeihyloctacosa-19,22-dien-9-amine, (18Z,21 Z)—N,N-dimethylheptacosa-18,21-dien-8-amine, (17Z,20Z)—N,N-dimethylhexacosa-17,20-dien-7-amine, (16Z,19Z)—N,N-dimethylpentacosa-16, 19-dien-6-amine, (22Z,25Z)—N,N-dimethylhentriaconta-22,25-dien-10-amine, (21 Z,24Z)—N,N-dimethyltriaconta-21,24-dien-9-amine, (18Z)—N,N-dimetylheptacos-18-en-10-amine, (17Z)—N,N-dimethylhexacos-17-en-9-amine, (19Z,22Z)—N,N-dimethyloctacosa-19,22-dien-7-amine, N,N-dimethylheptacosan-10-amine, (20Z,23Z)-N-ethyl-N-methylnonacosa-20,23-dien-10-amine, 1-[(11Z,14Z)-1-nonylicosa-11,14-dien-1-yl]pyrrolidine, (20Z)—N,N-dimethylheptacos-20-en-10-amine, (15Z)—N,N-dimethyl eptacos-15-en-10-amine, (14Z)—N,N-dimethylnonacos-14-en-10-amine, (17Z)—N,N-dimethylnonacos-17-en-10-amine, (24Z)—N,N-dimethyltritriacont-24-en-10-amine, (20Z)—N,N-dimethylnonacos-20-en-10-amine, (22Z)—N,N-dimethylhentriacont-22-en-10-amine, (16Z)—N,N-dimethylpentacos-16-en-8-amine, (12Z,15Z)—N,N-dimethyl-2-nonylhenicosa-12,15-dien-1-amine, (13Z,16Z)—N,N-dimethyl-3-nonyldocosa-13, 16-dien-1-amine, N,N-dimethyl-1-[(1S,2R)-2-octylcyclopropyl]eptadecan-8-amine, 1-[(1S,2R)-2-hexylcyclopropyl]-N,N-dimethylnonadecan-10-amine, N,N-dimethyl-1-[(1S,2R)-2-octylcyclopropyl]nonadecan-10-amine, N,N-dimethyl-21-[(1S,2R)-2-octylcyclopropyl]henicosan-10-amine, N,N-dimethyl-1-[(1S,2S)-2-{[(1R,2R)-2-pentylcyclopropyl]methyl}cyclopropyl]nonadecan-10-amine, N,N-dimethyl-1-[(1S,2R)-2-octylcyclopropyl]hexadecan-8-amine, N,N-dimethyl-[(1R,2S)-2-undecylcyclopropyl]tetradecan-5-amine, N,N-dimethyl-3-{7-[(1S,2R)-2-octylcyclopropyl]heptyl}dodecan-1-amine, 1-[(1R,2S)-2-heptylcyclopropyl]-N,N-dimethyloctadecan-9-amine, 1-[(1S,2R)-2-decylcyclopropyl]-N,N-dimethylpentadecan-6-amine, N,N-dimethyl-1-[(1S,2R)-2-octylcyclopropyl]pentadecan-8-amine, R-N,N-dimethyl-1-[(9Z,12Z)-octadeca-9,12-dien-1-yloxy]-3-(octyloxy)propan-2-amine, S-N,N-dimethyl-1-[(9Z,12Z)-octadeca-9,12-dien-1-yloxy]-3-(octyloxy)propan-2-amine, 1-{2-[(9Z,12Z)-octadeca-9,12-dien-1-yloxy]-1-[(octyloxy)methyl]ethyl}pyrrolidine, (2S)—N,N-dimethyl-1-[(9Z,12Z)-octadeca-9,12-dien-1-yloxy]-3-[(5Z)-oct-5-en-1-yloxy]propan-2-amine, 1-{2-[(9Z,12Z)-octadeca-9,12-dien-1-yloxy]-1-[(octyloxy)methyl]ethyl}azetidine, (2S)-1-(hexyloxy)-N,N-dimethyl-3-[(9Z,12Z)-octadeca-9,12-dien-1-yloxy]propan-2-amine, (2S)-1-(heptyloxy)-N,N-dimethyl-3-[(9Z,12Z)-octadeca-9,12-dien-1-yloxy]propan-2-amine, N,N-dimethyl-1-(nonyloxy)-3-[(9Z,12Z)-octadeca-9,12-dien-1-yloxy]propan-2-amine, N,N-dimethyl-1-[(9Z)-octadec-9-en-1-yloxy]-3-(octyloxy)propan-2-amine; (2S)—N,N-dimethyl-1-[(6Z,9Z,12Z)-octadeca-6,9,12-trien-1-yloxy]-3-(octyloxy)propan-2-amine, (2S)-1-[(11Z,14Z)-icosa-11,14-dien-1-yloxy]-N,N-dimethyl-3-(pentyloxy)propan-2-amine, (2S)-1-(hexyloxy)-3-[(11Z,14Z)-icosa-11,14-dien-1-yloxy]-N,N-dimethylpropan-2-amine, 1-[(11Z,14Z)-icosa-11,14-dien-1-yloxy]-N,N-dimethyl-3-(octyloxy)propan-2-amine, 1-[(13Z,16Z)-docosa-13, 16-dien-1-yloxy]-N,N-dimethyl-3-(octyloxy)propan-2-amine, (2S)-1-[(13Z,16Z)-docosa-13, 16-dien-1-yloxy]-3-(hexyloxy)-N,N-dimethylpropan-2-amine, (2S)-1-[(13Z)-docos-13-en-1-yloxy]-3-(hexyloxy)-N,N-dimethylpropan-2-amine, 1-[(13Z)-docos-13-en-1-yloxy]-N,N-dimethyl-3-(octyloxy)propan-2-amine, 1-[(9Z)-hexadec-9-en-1-yloxy]-N,N-dimethyl-3-(octyloxy)propan-2-amine, (2R)—N,N-dimethyl-H (1-metoyloctyl)oxyl-3-[(9Z,12Z)-octadeca-9,12-dien-1-yloxy]propan-2-amine, (2R)-1-[(3,7-dimethyloctyl)oxy]-N,N-dimethyl-3-[(9Z,12Z)-octadeca-9,12-dien-1-yloxy]propan-2-amine, N,N-dimethyl-1-(octyloxy)-3-({8-[(1S,2S)-2-{[(1R,2R)-2-pentylcyclopropyl]methyl}cyclopropyl]octyl}oxy)propan-2-amine, N,N-dimethyl-1-{[8-(2-oclylcyclopropyl)octyl]oxy}-3-(octyloxy)propan-2-amine and (11E,20Z,23Z)—N,N-dimethylnonacosa-11,20,2-trien-10-amine, or a pharmaceutically acceptable salt or stereoisomer thereof.

In some embodiments, the nanoparticle comprises a lipid-polycation complex. The formation of the lipid-polycation complex may be accomplished by methods known in the art and/or as described in U.S. Patent Publication No. 20120178702, herein incorporated by reference in its entirety. As a non-limiting example, the polycation may include a cationic peptide or a polypeptide such as, but not limited to, polylysine, polyornithine and/or polyarginine. In some embodiments, the composition may comprise a lipid-polycation complex, which may further include a non-cationic lipid such as, but not limited to, cholesterol or dioleoyl phosphatidylethanolamine (DOPE).

In exemplary aspects, the nanoparticles comprise cationic lipids, and nucleic acid molecules are complexed with the cationic lipid via electrostatic interactions. In exemplary instances, the RNA are mRNA In exemplary aspects, the liposomes are prepared by mixing RNA and the cationic lipid at a RNA: cationic lipid ratio of about 1 to about 10 to about 1 to about 20 (e.g., about 1 to about 19, about 1 to about 18, about 1 to about 17, about 1 to about 16, about 1 to about 15, about 1 to about 14, about 1 to about 13, about 1 to about 12, about 1 to about 11). In exemplary instances, the liposomes are prepared by mixing RNA and the cationic lipid at a RNA: cationic lipid ratio of about 1 to about 15. As used herein, the term “nucleic acid molecule: cationic lipid ratio” is meant a mass ratio, where the mass of the nucleic acid molecule is relative to the mass of the cationic lipid. Also, in exemplary aspects, the term “nucleic acid molecule: cationic lipid ratio” is meant the ratio of the mass of the nucleic acid molecule, e.g., RNA, added to the liposomes comprising cationic lipids during the process of manufacturing the RNA NPs of the present disclosure.

In exemplary embodiments, the nanoparticle comprises a surface and an interior comprising (i) a core and (ii) at least two nucleic acid layers, optionally, more than two nucleic acid layers. In exemplary instances, each nucleic acid layer is positioned between a lipid layer, e.g., a cationic lipid layer. In exemplary aspects, the nanoparticles are multilamellar comprising alternating layers of nucleic acid and lipid. In exemplary embodiments, the nanoparticle comprises at least three nucleic acid layers, each of which is positioned between a cationic lipid bilayer. In exemplary aspects, the nanoparticle comprises at least four or five nucleic acid layers, each of which is positioned between a cationic lipid bilayer. In exemplary aspects, the nanoparticle comprises at least more than five (e.g., 6, 7, 8, 9, 10, 11, 12, 13, 14, 15, 16, 17, 18, 19, 20, or more) nucleic acid layers, each of which is positioned between a cationic lipid bilayer. As used herein the term “cationic lipid bilayer” is meant a lipid bilayer comprising, consisting essentially of, or consisting of a cationic lipid or a mixture thereof. Suitable cationic lipids are described herein. As used herein the term “nucleic acid layer” is meant a layer of the presently disclosed nanoparticle comprising, consisting essentially of, or consisting of a nucleic acid, e.g., RNA.

In various aspects, the presently disclosed nanoparticle comprises a positively-charged surface. In some instances, the positively-charged surface comprises a lipid layer, e.g., a cationic lipid layer. In various aspects, the outermost layer of the nanoparticle comprises a cationic lipid bilayer. Optionally, the cationic lipid bilayer comprises, consists essentially of, or consists of DOTAP. In various instances, the surface comprises a plurality of hydrophilic moieties of the cationic lipid of the cationic lipid bilayer. In some aspects, the core comprises a cationic lipid bilayer. In various instances, the core lacks nucleic acids, optionally, the core comprises less than about 0.5 wt % nucleic acid.

In exemplary instances, the nanoparticle is characterized by a zeta potential of about +40 mV to about +60 mV, e.g., about +40 mV to about +55 mV, about +40 mV to about +50 mV, about +40 mV to about +50 mV, about +40 mV to about +45 mV, about +45 mV to about +60 mV, about +50 mV to about +60 mV, about +55 mV to about +60 mV. In exemplary aspects, the nanoparticle has a zeta potential of about +45 mV to about +55 mV. The nanoparticle in various instances, has a zeta potential of about +50 mV. In various aspects, the zeta potential is greater than +30 mV or +35 mV. The zeta potential is one parameter which distinguishes the nanoparticles of the present disclosure and those described in Sayour et al., Oncoimmunology 6(1): e1256527 (2016).

In various aspects, the nucleic acid molecules incorporated into the lipid nanoparticles are RNA molecules, e.g., transfer RNA (tRNA), ribosomal RNA (rRNA), messenger RNA (mRNA). The RNA molecules are optionally mRNA. The RNA may be modified, e.g., nucleoside modified, or unmodified. In various aspects, the RNA is modified to reduce the innate immune response, optionally leading to improved mRNA stability (i.e., “silenced”). In some aspects, 100% of the RNA is modified or silenced. In some aspects, about 90% of the RNA is modified or silenced, about 80% of the RNA is modified or silenced, about 70% of the RNA is modified or silenced, about 60% of the RNA is modified or silenced, about 50% of the RNA is modified or silenced, about 40% of the RNA is modified or silenced, about 30% of the RNA is modified or silenced, about 20% of the RNA is modified or silenced, about 10% of the RNA is modified or silenced, or about 5% of the RNA is modified or silenced. In some aspects, the silenced mRNA comprises one or more signal peptides.

In some aspects, the RNA comprises pseudouridine, such as N1-methyl-pseudouridine. In other aspects, the RNA does not comprise pseudouridine, e.g., N1-methyl-pseudouridine. For instance, the disclosure contemplates use of RNA wherein all or part of the uracils are substituted with N1-methyl-pseudouridine. In other aspects, the RNA comprises uridine. In some aspects, 100% of the uracils may be substituted with N1-methyl-pseudouridine. In some aspects, about 90% of the uracils may be substituted with N1-methyl-pseudouridine, about 80% of the uracils may be substituted with N1-methyl-pseudouridine, about 70% of the uracils may be substituted with N1-methyl-pseudouridine, about 60% of the uracils may be substituted with N1-methyl-pseudouridine, about 50% of the uracils may be substituted with N1-methyl-pseudouridine, about 40% of the uracils may be substituted with N1-methyl-pseudouridine, about 30% of the uracils may be substituted with N1-methyl-pseudouridine, about 20% of the uracils may be substituted with N1-methyl-pseudouridine, about 10% of the uracils may be substituted with N1-methyl-pseudouridine, or about 5% of the uracils may be substituted with N1-methyl-pseudouridine.

In various aspects, the RNA comprises polyinosinic: polycytidylic acid (poly I:C).

In some aspects, the RNA molecules may comprise a 5′ cap, optionally including modified guanine nucleotides on the 5′ end of the RNA molecule (i.e., the RNA is “capped”). In some aspects, the RNA molecules may not comprise a 5′ cap (i.e., are not capped). In some aspects, the RNA molecules may be capped but not silenced. In some aspects, the RNA molecules may be silenced but not capped. In some aspects, the RNA molecules may be capped and silenced. In some aspects, the RNA molecules may be partially capped, wherein some but not all of the RNA molecules comprise a 5′ cap. For example, in some aspects, at least about 5% of the RNA molecules may comprise a 5′ cap. In some aspects, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of the RNA molecules may comprise a 5′ cap.

In some aspects, the RNA molecules may comprise a higher molecular weight (HMW) structure, although this is not required. In some aspects, the RNA molecules may form HMW structures within the liposomes or LNPs. In some aspects, HMW may comprise RNA structures greater than about 1.0 kb, including at least about 1.1 kb, at least about 1.2 kb, at least about 1.3 kb, at least about 1.4 kb, at least about 1.5 kb, at least about 1.6 kb, at least about 1.7 kb, at least about 1.8 kb, at least about 1.9 kb, or at least about 2.0 kb. In some aspects, the RNA molecules may comprise double stranded or single stranded RNA.

The RNA (e.g., mRNA) in exemplary aspects encodes a peptide or a protein. Optionally, the protein is selected from the group consisting of an antigen, a cytokine, and a co-stimulatory molecule. Indeed, the protein is, in some aspects, selected from the group consisting of an antigen, a co-stimulatory molecule, a cytokine, a growth factor, a hematopoietic factor, or a lymphokine, including, e.g., cytokines and growth factors that are effective in inhibiting tumor metastasis, and cytokines or growth factors that have been shown to have an antiproliferative effect on at least one cell population. In some aspects, the protein may be a tumor protein, such as a tumor protein derived from the subject undergoing treatment (a personalized tumor protein) or a tumor protein which is not derived from the subject undergoing treatment.

In some aspects, the protein is not expressed by a tumor cell or by a human. For instance, the protein is optionally not related to a tumor antigen or cancer antigen (i.e., the protein is not a tumor antigen). In this regard, the mRNA may encode a protein that is non-specific relative to a tumor or cancer. For example, the protein may be green fluorescence protein (GFP) or ovalbumin (OVA). The protein (i.e., non-tumor protein) may be an antigen which triggers an immune response against a non-cancer target, such as a bacteria or virus. In some aspects, the protein may comprise a viral antigen. Viral antigens are well known in the art and may be derived from, e.g., noroviruses, rotaviruses, adenoviruses, astroviruses, herpes viruses, retroviruses, flavivirid viruses, picoviruses, polyoma viruses, toroviruses, coronaviruses (including severe acute respiratory syndrome virus (SARS), such as SARS-CoV-2, or middle east respiratory syndrome-related coronavirus (MERS)), exanthematous poxviruses (e.g., chickenpox, smallpox, or varicella-zoster), hemorrhagic viruses, neurological viruses (e.g., polio, viral meningitis, viral encephalitis, and rabies) and the like. In various aspects, the viral antigen is a coronavirus antigen, an influenza antigen, HIV antigen, malaria antigen, and/or tuberculosis antigen. In various aspects, the mRNA encodes a SARS-CoV-2 protein, such as spike protein. Alternatively, the mRNA may encode pp65 from human cytomegalovirus (CMV).

The disclosure provides a method wherein the lipid nanoparticle comprises nucleoside-modified messenger RNA (mRNA) encoding the pre-fusion stabilized Spike glycoprotein(S) of the SARS-CoV-2 Omicron variant lineage KP.2, SM-102, polyethylene glycol [PEG] 2000 dimyristoyl glycerol [DMG], cholesterol, and 1,2-distearoyl-sn-glycero-3-phosphocholine [DSPC]. For instance, the RNA lipid nanoparticle is, in various aspects SPIKEVAX™. The RNA nanoparticle may alternatively comprise nucleoside-modified messenger RNA encoding the viral spike (S) glycoprotein of SARS-CoV-2 Omicron variant lineage KP.2, ((4-hydroxybutyl)azanediyl)bis(hexane-6,1-diyl)bis(2-hexyldecanoate), 2-(polyethylene glycol 2000)-N,N-ditetradecylacetamide, 1,2-distearoyl-sn-glycero-3-phosphocholine, and cholesterol. For instance, the RNA lipid nanoparticle is, in various aspects COMIRNATY™.

The disclosure also contemplates aspects wherein the RNA is tumor RNA, i.e., the sequence of RNA mirrors that found in human tumors or encodes a protein found in human tumors. In various aspects, the nanoparticle comprises a mixture of RNA which is RNA isolated from a tumor of a human. Optionally, the RNA is in vitro transcribed mRNA, wherein the in vitro transcription template is cDNA made from RNA extracted from a tumor cell. In exemplary aspects, the mRNA encodes a tumor antigen, such as an antigen derived from a viral protein, an antigen derived from point mutations, or an antigen encoded by a cancer-germline gene. In exemplary aspects, the tumor antigen is p53, KRAS, NRAS, MAGEA, MAGEB, MAGEC, BAGE, GAGE, LAGE/NY-ESO1, SSX, tyrosinase, gp100/pmel17, Melan-A/MART-1, gp75/TRP1, TRP2, CEA, RAGE-1, HER2/NEU, or WT1.

The nanoparticles described herein are optionally provided in a composition, such as a pharmaceutical composition. In exemplary aspects, the composition is a pharmaceutical composition comprising a plurality of nanoparticles according to the present disclosure and a pharmaceutically acceptable carrier, diluent, or excipient and intended for administration to a human. In exemplary aspects, the composition is a sterile composition.

In various aspects, the composition comprising the nanoparticles does not comprise cells, e.g., dendritic cells. In various aspects, the composition comprising the nanoparticles does not comprise microorganisms, such as bacteria or viruses.

In exemplary aspects, the composition of the present disclosure may comprise additional components other than the nanoparticle, a cell comprising the nanoparticle, or population of cells. The composition, in various aspects, comprises any pharmaceutically acceptable ingredient, including, for example, acidifying agents, additives, adsorbents, aerosol propellants, air displacement agents, alkalizing agents, anticaking agents, anticoagulants, antimicrobial preservatives, antioxidants, antiseptics, bases, binders, buffering agents, chelating agents, coating agents, coloring agents, desiccants, detergents, diluents, disinfectants, disintegrants, dispersing agents, dissolution enhancing agents, dyes, emollients, emulsifying agents, emulsion stabilizers, fillers, film forming agents, flavor enhancers, flavoring agents, flow enhancers, gelling agents, granulating agents, humectants, lubricants, mucoadhesives, ointment bases, ointments, oleaginous vehicles, organic bases, pastille bases, pigments, plasticizers, polishing agents, preservatives, sequestering agents, skin penetrants, solubilizing agents, solvents, stabilizing agents, suppository bases, surface active agents, surfactants, suspending agents, sweetening agents, therapeutic agents, thickening agents, tonicity agents, toxicity agents, viscosity-increasing agents, water-absorbing agents, water-miscible cosolvents, water softeners, or wetting agents. See, e.g., the Handbook of Pharmaceutical Excipients, Third Edition, A. H. Kibbe (Pharmaceutical Press, London, U K, 2000), which is incorporated by reference in its entirety. Remington's Pharmaceutical Sciences, Sixteenth Edition, E. W. Martin (Mack Publishing Co., Easton, Pa., 1980), which is incorporated by reference in its entirety.

The composition of the present disclosure (e.g., the nanoparticle composition and/or an ICI composition) can be suitable for administration by any acceptable route. Systemic routes of administration are contemplated, while localized delivery may alternatively (or additionally) be performed. Exemplary routes of administration of an active agent to a subject include, but are not limited to, intravenous, intradermal, intramuscular, subcutaneous, intraperitoneal, intranodal, and intrasplenic administration. Optionally, the nanoparticle is administered intramuscularly. In various aspects, the lipid nanoparticle composition is administered to the subject on the same side of the body as the tumor (i.e., the lipid nanoparticle composition is administered ipsilaterally with respect to the tumor). The disclosure also contemplates contralateral administration.

Without being bound to any particular theory, the data provided herein support the use of the RNA nanoparticles for increasing an immune response against a tumor in a subject. Accordingly, a method of increasing an immune response against a tumor in a subject is provided by the present disclosure. In exemplary embodiments, the method comprises administering to the subject a pharmaceutical composition comprising RNA nanoparticles. Optionally, the composition is systemically administered to the subject. For example, the composition is administered intravenously or intramuscularly. The method also optionally comprises administering an ICI to the subject. In this regard, the disclosure also provides a method of treating a tumor in a subject, the method comprising administering to the subject a pharmaceutical composition comprising RNA nanoparticles and also administering to the subject an ICI.

In various aspects, the tumor is refractory to immune checkpoint therapy prior to administration of the composition comprising RNA nanoparticles, i.e., one or more ICIs has reduced efficacy in eliciting an immune response against the tumor prior to administration of the nanoparticles described herein. Alternatively, the tumor is not refractory, but the method further enhances sensitivity to the immune response such that enhanced tumor cell death is achieved.

The present disclosure provides a method of increasing sensitivity of a tumor to treatment with an immune checkpoint inhibitor (ICI) in a subject. In exemplary embodiments, the method comprises administering to the subject a composition comprising a nanoparticle described herein. Optionally, the method further comprises administering an ICI to the subject.

The present disclosure further provides a method of treating a subject with an immune checkpoint inhibitor (ICI)-resistant tumor. In exemplary embodiments, the method comprises administering to the subject (a) a composition comprising a nanoparticle described herein and (b) an ICI. Optionally, the nanoparticle composition is systemically administered to the subject (e.g., via intramuscular injection).

An “immune checkpoint inhibitor” or “ICI” is any agent (e.g., compound or molecule) that that decreases, blocks, inhibits, abrogates or interferes with the function of a protein of an immune checkpoint pathway. Proteins of the immune checkpoint pathway regulate immune responses and, in some instances, prevent T cells from attacking cancer cells. In various aspects, the protein of the immune checkpoint pathway is, for example, CTLA-4, PD-1, PD-L1, PD-L2, B7-H3, B7-H4, TIGIT, VISTA, LAG3, CD112 TIM3, BTLA, or co-stimulatory receptor ICOS, OX40, 41BB, or GITR. In various aspects, the ICI is a small molecule, an inhibitory nucleic acid, or an inhibitor polypeptide. In various aspects, the ICI is an antibody, antigen-binding antibody fragment, or an antibody protein product that binds to and inhibits the function of the protein of the immune checkpoint pathway.

Suitable ICIs which are antibodies, antigen-binding antibody fragments, or an antibody protein products are known in the art and include, but are not limited to, ipilimumab (CTLA-4; Bristol Meyers Squibb), nivolumab (PD-1; Bristol Meyers Squibb), pembrolizumab (PD-1; Merck), atezolizumab (PD-L1; Genentech), avelumab (PD-L1; Merck), and durvalumab (PD-L1; Medimmune) (Wei et al., Cancer Discovery 8:1069-1086 (2018)). Other examples of ICIs include, but are not limited to, IMP321 (LAG3: Immuntep); BMS-986016 (LAG3; Bristol Meyers Squibb); IPH2101 (KIR; Innate Pharma); tremelimumab (CTLA-4; Medimmune); pidilizumab (PD-1; Medivation); MPDL3280A (PD-L1; Roche); MEDI4736 (PD-L1; AstraZeneca); MSB0010718C (PD-L1; EMD Serono); AUNP12 (PD-1; Aurigene); MGA271 (B7-H3: MacroGenics); and TSR-022 (TIM3; Tesaro).

In various aspects, the ICI is a PD-L1 inhibitor. Programmed death-ligand 1 (PD-L1; also known as cluster of differentiation 274 (CD274) or B7 homolog 1 (B7-H1)) is a transmembrane protein that functions to suppress the immune system in, e.g., pregnancy, tissue allografts, and autoimmune disease. Binding of PD-L1 to its receptor PD-1 transmits an inhibitory signal that reduces the proliferation and function of T cells and can induce apoptosis. For example, the PD-L1 inhibitor binds to and inhibits the function of PD-L1. In various aspects, the PD-L1 inhibitor is an anti-PD-L1 antibody, antigen binding antibody fragment, or an antibody-like molecule.

In various aspects, the ICI is a PD-1 inhibitor. “Programmed Death-1” (PD-1), also known as cluster of differentiation 279 (CD279), refers to an immunoinhibitory receptor belonging to the CD28 family. PD-1 is expressed on previously activated T cells in vivo, and binds to two ligands, PD-L1 and PD-L2. The human PD-1 sequence can be found under GenBank Accession No. U64863. For example, the PD-1 inhibitor binds to and inhibits the function of PD-1, e.g., an anti-PD-1 antibody, antigen binding antibody fragment, or an antibody-like molecule. In various aspects, the PD-1 inhibitor is durvalumab, atezolizumab, or avelumab. In various aspects, the ICI is a PD-L2 inhibitor. For example, the PD-L2 inhibitor binds to and inhibits the function of PD-L2, e.g., an anti-PD-L2 antibody, antigen binding antibody fragment, or an antibody-like molecule.

Examples of PD-1 and PD-L1 inhibitors are described in, e.g., U.S. Pat. Nos. 7,488,802; 7,943,743; 8,008,449; 8,168,757; 8,217,149; and PCT Patent Publication Nos. WO03042402, WO2008156712, WO2010089411, WO2010036959, WO2011066342, WO2011159877, WO2011082400, and WO2011161699; which are incorporated by reference herein in their entireties.

Cytotoxic T-lymphocyte-associated protein 4 (CTLA-4, also known as CD152), is a membrane protein expressed on T cells and regulatory T cells (Treg). CTLA-4 binds B7-1 (CD80) and B7-2 (CD86) on antigen-presenting cells (APC), which inhibits the adaptive immune response. In humans, CTLA-4 is encoded in various isoforms; an exemplary amino acid sequence is available as GenBank Accession No. NP_001032720. A representative anti-CTLA-4 antibody is ipilimumab (YERVOY®, Bristol-Myers Squibb).

The term “inhibit” and words stemming therefrom does not require a 100% or complete inhibition or abrogation. Rather, there are varying degrees of inhibition of which one of ordinary skill in the art recognizes as having a potential benefit or therapeutic effect. In exemplary embodiments, the inhibition provided by the methods is at least or about a 10% inhibition (e.g., at least or about a 20% inhibition, at least or about a 30% inhibition, at least or about a 40% inhibition, at least or about a 50% inhibition, at least or about a 60% inhibition, at least or about a 70% inhibition, at least or about a 80% inhibition, at least or about a 90% inhibition, at least or about a 95% inhibition, at least or about a 98% inhibition). The ICIs may inhibit the function of an immune checkpoint protein to any level.

As used herein “sensitivity” refers to the way a tumor reacts to a drug/compound, e.g., a ICI inhibitor (e.g., PD-L1 inhibitor). In exemplary aspects, “sensitivity” means “responsive to treatment” and the concepts of “sensitivity” and “responsiveness” are positively associated in that a tumor or cancer cell that is responsive to a drug/compound treatment is said to be sensitive to that drug. “Sensitivity” in exemplary instances is defined according to Pelikan, Edward, Glossary of Terms and Symbols used in Pharmacology (Pharmacology and Experimental Therapeutics Department Glossary at Boston University School of Medicine), as the ability of a population, an individual or a tissue, relative to the abilities of others, to respond in a qualitatively normal fashion to a particular drug dose. The smaller the dose required producing an effect, the more sensitive is the responding system. In exemplary aspects, “sensitivity” is opposite to “resistant” and the concept of “resistance” is negatively associated with “sensitivity”. For example, a tumor that is resistant to a drug treatment is neither sensitive nor responsive to that drug, and that drug is not an effective treatment for that tumor or cancer cell. In the context of ICI's, a tumor which is insensitive to ICIs is one which does not respond to ICI therapy in a clinically significant way. Improving the sensitivity of a tumor to an ICI encompasses, e.g., any improvement in the clinical responsiveness to ICI therapy, which may be detected by a reduction in tumor volume or increase in tumor cell death, a reduction in the dose of ICI required to achieve a clinically detectable response, an increase in the time interval between ICI doses (requiring less frequent dosing) while maintaining a clinically detectable response, and the like. “Sensitivity” also is used herein with respect to a host immune response. In this respect, a tumor which evades a host immune response is “resistant” (or refractory). A tumor that is “sensitive” to a host immune response is recognized by the host immune system and subject to attack by immune effector cells. A tumor that is “sensitive” to a host immune response is recognized by the host immune system and subject to attack by immune effector cells.

As used herein, the term “increase” and words stemming therefrom may not be a 100% or complete increase. Rather, there are varying degrees of increasing of which one of ordinary skill in the art recognizes as having a potential benefit or therapeutic effect. In exemplary embodiments, the increase provided by the methods is at least or about a 10% increase (e.g., at least or about a 20% increase, at least or about a 30% increase, at least or about a 40% increase, at least or about a 50% increase, at least or about a 60% increase, at least or about a 70% increase, at least or about a 80% increase, at least or about a 90% increase, at least or about a 95% increase, at least or about a 98% increase). In various aspects, the “increase” is in reference to baseline measurements (e.g., baseline immunity, sensitivity, or activation) in the absence of (e.g., prior to) administering the nanoparticles of the instant disclosure.

In the context of the disclosure, administration of the nanoparticles of the disclosure sensitizes a tumor to an ICI, and together the two active agents increase the sensitivity of the tumor to a host immune response.

Increased sensitivity to an ICI or increased sensitivity to host immune response may be determined in any of a number of ways. For example, administration of the RNA nanoparticles and ICI may increase the number of cytotoxic T cells in a tumor and/or enhance cytotoxic T cell activity. For example, in various embodiments, the method may increase perforin, IFN-gamma, and/or granzyme production by cytotoxic T cells and increase cytolytic activity. Further, the method described herein may enhance T cell survival, promote T cell longevity, and/or restrict loss of replicative potential. Methods of measuring T cell activity and immune responses are known in the art. T cell activity can be measured by, for example, a cytotoxicity assay, such as those described in Fu et al., PLoS ONE 5 (7): e11867 (2010). Other T cell activity assays are described in Bercovici et al., Clin Diagn Lab Immunol. 7 (6): 859-864 (2000). Methods of measuring immune responses are described in e.g., Macatangay et al., Clin Vaccine Immunol 17 (9): 1452-1459 (2010), and Clay et al., Clin Cancer Res.7 (5): 1127-35 (2001). In various aspects, the method of the disclosure enhances cytotoxic T cell mediated killing of cancer cells within the tumor.

The methods of the present disclosure may comprise the above described step(s) alone or in combination with other steps. The methods may comprise repeating any one of the above-described step(s) and/or may comprise additional steps, aside from those described above. For instance, the presently disclosed methods may further comprise obtaining a sample of the tumor of the subject, optionally, via a biopsy. In various aspects, the disclosure provides a method comprising administering to a subject a lipid nanoparticle described herein (which optionally comprises mRNA that encodes a protein which is not a tumor antigen), and performing a biopsy. In various aspects, the lipid nanoparticle described herein administered prior to biopsy, e.g., within about 100 days prior to biopsy (such as 51-100 days prior to biopsy or 30-100 days prior to biopsy or within 30 days of biopsy).

In exemplary aspects, the method comprises administering an ICI to the subject. In this regard, the present disclosure further provides a method of treating a subject with an immune checkpoint inhibitor (ICI)-resistant tumor, although this is not required. In exemplary aspects, the method comprises administering to the subject (a) an RNA nanoparticle, and (b) a PD-L1 inhibitor. Optionally, the RNA nanoparticle is systemically administered to the subject (e.g., via intramuscular administration). In exemplary aspects, the PD-L1 inhibitor is a PD-L1 antibody. PD-L1 inhibitors are known in the art and include, but are not limited to, atezolizumab, avelumab, and durvalumab.

As used herein, the term “treat,” as well as words related thereto, do not necessarily imply 100% or complete treatment or remission. Rather, there are varying degrees of treatment of which one of ordinary skill in the art recognizes as having a potential benefit or therapeutic effect. In this respect, the methods of treating a disease of the present disclosure can provide any amount or any level of treatment. Furthermore, the treatment provided by the method may include treatment of one or more conditions or symptoms or signs of the disease being treated. For instance, the treatment method of the presently disclosure may inhibit one or more symptoms of the disease. Also, the treatment provided by the methods of the present disclosure may encompass slowing the progression of the disease. For example, the methods can treat cancer by virtue of enhancing the T cell activity or an immune response against the cancer, thereby reducing tumor or cancer growth, reducing metastasis of tumor cells, increasing cell death of tumor or cancer cells, and the like

The term “treat” also encompasses prophylactic treatment of the disease. Accordingly, the treatment provided by the presently disclosed method may delay the onset or reoccurrence/relapse of the disease being prophylactically treated. In exemplary aspects, the method delays the onset of the disease by 1 day, 2 days, 4 days, 6 days, 8 days, 10 days, 15 days, 30 days, two months, 4 months, 6 months, 1 year, 2 years, 4 years, or more. The prophylactic treatment encompasses reducing the risk of the disease being treated. In exemplary aspects, the method reduces the risk of the disease 2-fold, 5-fold, 10-fold, 20-fold, 50-fold, 100-fold, or more.

In certain aspects, the method of treating the disease may be regarded as a method of inhibiting the disease, or a symptom thereof. As used herein, the term “inhibit” and words stemming therefrom may not be a 100% or complete inhibition or abrogation. Rather, there are varying degrees of inhibition of which one of ordinary skill in the art recognizes as having a potential benefit or therapeutic effect. The presently disclosed methods may inhibit the onset or re-occurrence of the disease or a symptom thereof to any amount or level. In exemplary embodiments, the inhibition provided by the methods is at least or about a 10% inhibition (e.g., at least or about a 20% inhibition, at least or about a 30% inhibition, at least or about a 40% inhibition, at least or about a 50% inhibition, at least or about a 60% inhibition, at least or about a 70% inhibition, at least or about a 80% inhibition, at least or about a 90% inhibition, at least or about a 95% inhibition, at least or about a 98% inhibition).

The susceptibility of a tumor to an immune response (or ICI) or, put another way, the effectiveness of an immune response (or ICI) against a tumor, can be determined in a variety of ways. Similarly, treatment a subject for cancer may be determined by any of a number of ways. Any improvement in the subject's well being is contemplated (e.g., at least or about a 10% reduction, at least or about a 20% reduction, at least or about a 30% reduction, at least or about a 40% reduction, at least or about a 50% reduction, at least or about a 60% reduction, at least or about a 70% reduction, at least or about a 80% reduction, at least or about a 90% reduction, or at least or about a 95% reduction of any parameter described herein). For example, a therapeutic response would refer to one or more of the following improvements in the disease: (1) a reduction in the number of neoplastic cells; (2) an increase in neoplastic cell death; (3) inhibition of neoplastic cell survival; (4) inhibition (i.e., slowing to some extent, preferably halting) of tumor growth or appearance of new lesions; (5) decrease in tumor size or burden; (6) absence of clinically detectable disease, (7) decrease in levels of cancer markers; (8) an increased patient survival rate; and/or (9) some relief from one or more symptoms associated with the disease or condition (e.g., pain). For example, the efficacy of treatment may be determined by detecting of a change in tumor mass and/or volume after treatment. The size of a tumor may be compared to the initial size and dimensions as measured by CT, PET, mammogram, ultrasound, or palpation, as well as by caliper measurement or pathological examination of the tumor after biopsy or surgical resection. Response may be characterized quantitatively using, e.g., percentage change in tumor volume (e.g., the method of the disclosure results in a reduction of tumor volume by at least 10%, at least 20%, at least 30%, at least 40%, at least 50%, at least 60%, at least 70%, at least 80%, or at least 90%). Alternatively, tumor response or cancer response may be characterized in a qualitative fashion like “pathological complete response” (pCR), “clinical complete remission” (cCR), “clinical partial remission” (cPR), “clinical stable disease” (cSD), “clinical progressive disease” (cPD), or other qualitative criteria. In addition, treatment efficacy also can be characterized in terms of responsiveness to other immunotherapy treatment or chemotherapy. In various aspects, the methods of the disclosure further comprise monitoring treatment in the subject.

The amount or dose of an active agent (i.e., the “effective amount”) administered should be sufficient to achieve a desired biological effect, e.g., a therapeutic or prophylactic response, in the subject over a reasonable time frame. For example, one or more doses of the nanoparticles described herein and ICI should be sufficient to, e.g., sensitize a tumor to an immune response (and optionally treat a cancer) in a clinically acceptable period of time e.g., 1 to 20 or more weeks, from the time of first administration. In certain embodiments, the time period could be even longer. By way of example and not intending to limit the present disclosure, the dose of the active agents of the present disclosure can be about 0.0001 to about 1 g/kg body weight of the subject being treated/day, from about 0.0001 to about 0.001 g/kg body weight, or about 0.01 mg to about 1 g/kg body weight.

In instances wherein the method comprises administering a nanoparticle of the disclosure and an ICI to a subject, the nanoparticle composition and ICI may be administered together (in the same formulation or separate formulations administered close in time) or may be administered sequentially (i.e., the nanoparticle composition is administered and the ICI is administered separately at different time points (e.g., hours or days apart)).

The nanoparticle composition of the disclosure is optionally administered prior to the ICI, e.g., at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, at least about 30 days, at least about 35 days, at least about 42 days, at least about 50 days, at least about 60 days, at least about 70 days, at least about 80 days, at least about 90 days, or at least about 100 days prior to ICI administration. In various aspects, the nanoparticle composition of the disclosure is administered no more than 100 days prior to the ICI, e.g., no more than 90 days, no more than 80 days, no more than 70 days, or no more than 60 days prior to the ICI, no more than 50 days prior to the ICI, no more than 40 days prior to the ICI, no more than 30 days prior to the ICI, no more than 20 days prior to the ICI, or no more than 10 days prior to the ICI. To illustrate, the nanoparticle composition may be administered within about 100 days of ICI administration, such as administration about 51-100 days prior to ICI administration. In various aspects, the ICI may be administered within about 30 days of nanoparticle administration. For example, the method may, in various instances, comprise a first period of nanoparticle treatment followed by a second period of ICI treatment. The second period of ICI treatment may also entail treatment with the nanoparticles to enhance the immune response (e.g., the second period may comprise both ICI administration and nanoparticle administration, separately or in combination). The first period of nanoparticle administration may entail multiple doses of nanoparticles administered to the subject over time, e.g., two, three, four, five, or more doses.

Alternatively, the ICI is optionally administered prior to the nanoparticle composition of the disclosure, e.g., at least about 21 days, at least about 28 days, at least about 30 days, at least about 35 days, at least about 42 days, at least about 50 days, at least about 60 days, at least about 70 days, at least about 80 days, at least about 90 days, or at least about 100 days prior to administration of the nanoparticle composition of the disclosure. In various aspects, the ICI is administered no more than 100 days prior to the nanoparticle composition of the disclosure, e.g., no more than 90 days, no more than 80 days, no more than 70 days, no more than 60 days prior to the nanoparticle composition of the disclosure, no more than 50 days, no more than 40 days, no more than 30 days, no more than 20 days, or no more than 10 days prior to the ICI. To illustrate, the ICI may be administered within about 100 days of nanoparticle composition administration, such as ICI administration about 1-30 days, about 20-50 days, or 51-100 days prior to nanoparticle composition administration. For example, the method may, in various instances, comprise a first period of ICI treatment followed by a second period of lipid nanoparticle treatment. The second period of nanoparticle treatment may also entail treatment with the ICI.

The subject of the instant disclosure is a mammal, including, but not limited to, mammals of the order Rodentia, such as mice and hamsters, and mammals of the order Logomorpha, such as rabbits, mammals from the order Carnivora, including Felines (cats) and Canines (dogs), mammals from the order Artiodactyla, including Bovines (cows) and Swines (pigs) or of the order Perssodactyla, including Equines (horses). In some aspects, the mammals are of the order Primates, Ceboids, or Simoids (monkeys) or of the order Anthropoids (humans and apes). In some aspects, the mammal is a human.

The subject may be one who has been previously diagnosed with or identified as suffering from or having a condition in need of treatment (e.g., cancer) or one or more complications related to such a condition, and optionally, have already undergone treatment for the condition or the one or more complications related to the condition. Alternatively, a subject can also be one who has not been previously diagnosed as having such condition or related complications. For example, a subject can be one who exhibits one or more risk factors for the condition or one or more complications related to the condition. The subject, in various aspects, has previously received a treatment or therapy for the condition (e.g., previously been administered an anti-cancer therapy, such as an ICI). In some aspects, the subject may have low expression of PD-L1 pre-treatment (e.g., at baseline) including less than about 10%, less than about 9%, less than about 8%, less than about 7%, less than about 6%, less than about 5%, less than about 4%, less than about 3%, less than about 2%, or less than about 1%. In some aspects, the subject may have PD-L1 expression between 1% and 50% pre-treatment. In some aspects, the subject may have PD-L1 expression no more than 50% pre-treatment. In some aspects, the subject may have recurrent disease. In some aspects, the subject may not have received previously received a COVID vaccine (or other type of non-cancer specific vaccine) but may have recurrent disease.

The cancer treatable by the methods disclosed herein may be any cancer, e.g., any malignant growth or tumor caused by abnormal and uncontrolled cell division that may spread to other parts of the body through the lymphatic system or the blood stream.

The cancer in some aspects is one selected from the group consisting of acute lymphocytic cancer, acute myeloid leukemia, alveolar rhabdomyosarcoma, bone cancer, brain cancer (e.g., glioma), breast cancer (e.g., triple negative breast cancer), cancer of the anus, anal canal, or anorectum, cancer of the eye, cancer of the intrahepatic bile duct, cancer of the joints, cancer of the head, neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, gastrointestinal cancer (e.g., gastrointestinal carcinoid tumor), Hodgkin lymphoma, endometrial or hepatocellular carcinoma, hypopharynx cancer, kidney cancer, larynx cancer, liver cancer, lung cancer (e.g., non-small cell lung cancer, bronchioloalveolar carcinoma), malignant mesothelioma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer (e.g., renal cell carcinoma (RCC)), small intestine cancer, soft tissue cancer, stomach cancer, testicular cancer, thyroid cancer, ureter cancer, and urinary bladder cancer. In particular aspects, the cancer is selected from the group consisting of head and neck, ovarian, cervical, bladder and oesophageal cancers, pancreatic, gastrointestinal cancer, gastric, breast, endometrial and colorectal cancers, hepatocellular carcinoma, glioblastoma, bladder, and lung cancer (e.g., non-small cell lung cancer (NSCLC), bronchioloalveolar carcinoma). In various aspects, the subject has a solid tumor. Optionally, the subject suffers from a malignant brain tumor, such as a glioblastoma, medulloblastoma, diffuse intrinsic pontine glioma, or a peripheral tumor with metastatic infiltration into the central nervous system. In various aspects, the subject suffers from non-small cell lung cancer (NSCLC). In various aspects, the subject suffers from melanoma.

The following examples are given merely to illustrate the invention and not in any way to limit its scope.

### EXAMPLE

This example describes studies establishing that mRNA nanoparticle vaccines (e.g., SARS-CoV-2 mRNA vaccines) sensitize tumors to immune checkpoint blockade.

### Methods

Retrospective Studies: A non-interventional, retrospective review of patient data was completed using the MD Anderson Cancer Center (MDACC) electronic health record (EHR) system, which contains a record of the patients who are treated at the primary campus of MDACC, a large quaternary cancer hospital in Houston, Texas. The chart review for this study involved three groups of patients: (a) patients diagnosed or treated with Stage III or Stage IV non-small cell lung cancer (NSCLC) between 11/2019 and 11/2023 who did or did not receive immune checkpoint inhibition, (b) patients diagnosed or treated with melanoma of any stage between January 2019 and November 2023 who received frontline single or multi-agent immune checkpoint blockade (nivolumab, ipilimumab, pembrolizumab, nivolumab+ipilimumab etc.), and (c) a tissue agnostic cohort, which included all patients with pathology results for PD-L1 since August 2020 at our institution across a wide range of histologies. MD Anderson Cancer Center institutional review board approval for this study was obtained for each group of patients individually, and informed consent was waived due to the retrospective and de-identified nature of the data. The data collection cutoff was May 1, 2024; data analysis and retrospective chart review were performed from Mar. 1, 2024 to May 1, 2024.

In the non-small cell lung cancer dataset, patient information was collected regarding patient demographics (age at immunotherapy start, sex, ethnicity, etc.), primary tumor histology, clinical stage, tumor mutations (EGFR, KRAS, HER2, ALK, MET, p53, RET, etc.), metastatic burden at immunotherapy start (brain, bone, liver, overall metastases), Eastern Cooperative Oncology Group (ECOG) performance status (PS; range, 0-5) at the initiation of immunotherapy, radiation therapy in the time around immunotherapy start, surgical and chemotherapy history, immunodeficiency, comorbidities (heart disease, kidney disease, liver disease, respiratory disease), other primary tumors, steroid usage around immunotherapy initiation, date of last follow-up, date of death, date of first recurrence/progression, immune checkpoint inhibition (ICI) agent names and start dates, COVID-19, influenza, pneumococcal vaccination dates, and tumor proportion score (TPS).

In the melanoma dataset, patient information was collected regarding patient demographics (age at immunotherapy start, sex, ethnicity, etc), primary tumor histology, clinical stage, tumor mutations (EGFR, KRAS, HER2, ALK, MET, p53, RET, etc.), metastatic burden at immunotherapy start (brain, bone, liver, overall metastases), performance status (PS; range, 0-5) at the initiation of immunotherapy, radiation therapy in the time around immunotherapy start, surgical and chemotherapy history, immunodeficiency, comorbidity (heart disease, kidney disease, liver disease, respiratory disease), other primary tumor data, steroid usage around COVID vaccination and immunotherapy initiation, date of last follow-up, date of death, date of first recurrence/progression, immune checkpoint inhibition (ICI) agent names and start dates, and COVID-19 vaccination dates.

Patients in both the NSCLC and melanoma datasets were separated into two groups: (i) patients who received a COVID mRNA vaccination within 100 days of ICI start, and (ii) patients who did not receive COVID mRNA vaccination. Survival analysis was performed using these groups, with sub-analysis involving staging of the tumor, brand of mRNA vaccine, number of doses of the COVID vaccine, location of metastases, and cycle of immunotherapy (first-line ICI, second-line ICI, etc.)

For patients who received COVID-19 mRNA vaccination in both the NSCLC and melanoma datasets, overall survival was calculated as the time between the date of immunotherapy start closest to mRNA vaccination date, and the last follow-up date (for patients who are living) or date of death. For patients who did not receive COVID-19 mRNA vaccination, overall survival was calculated as the time between the initiation of their first ICI start and the date of death or last follow-up. For patients who received COVID mRNA vaccination in both datasets, progression-free survival was calculated as the time between the initiation of ICI closest to mRNA vaccination and the first incidence of either pathology-confirmed recurrence or imaging-confirmed progression, whichever occurred earlier, that was declared progression in their primary medical oncologist's clinical notes. For patients who did not receive COVID mRNA vaccination, progression-free survival was calculated as the time between their first ICI start date and clinician-confirmed progression as described above. Patients who progressed prior to the receipt of mRNA vaccination were included in the non-vaccination group for this analysis, even if they ultimately received an mRNA vaccine within 100 days of initiation of ICI but after progression. Kaplan-Meier curves were generated using GraphPad Prism.

Cox Proportional Hazards Regression: For Cox proportional hazards regression, time-dependent variables were defined as follows: “Steroid use within 1 month of ICI start” indicated whether a patient received dexamethasone, prednisone, or methylprednisolone within 1 month before or after immunotherapy start date. “Radiation immediately before ICI” describes whether a patient received radiation within 90 days before ICI, while “Radiation with ICI” describes receipt of radiation within 60 days after the start of ICI. “Surgery immediately before ICI” describes any tumor resection surgery prior to the initiation of ICI. “Gender” was described as a binary variable between 0 (Female) and 1 (Male). “Race” was described as a binary variable between White, 1, and other, 0 in this dataset, due to low quantities of non-white populations. Statistical analysis was completed using GraphPad Prism.

Quantification of PD-L1 Expression in Biopsy Samples: For patients in the tissue agnostic cohort, patient PD-L1 biopsy date, pathology reports, histologic information, and diagnosis codes were collected for patients who have received a PD-L1 biopsy at MD Anderson. From the biopsy report, PD-L1 CPS and TPS were manually entered into a large dataset, and data was curated according to the following principles: (i) simple quantitative TPS and CPS values were replicated exactly, (ii) TPS or CPS values of “<1%” were reported as 0%, (iii) for patients with a CPS of 0% with no TPS value, TPS was reported as 0%, as CPS of 0 implies a lack of expression of PD-L1 on both tumor cells and myeloid cells, (iv) TPS and CPS values reported as “>x %” were excluded. For the tissue agnostic cohort, patients were separated into groups by organ system (i.e., thoracic, gastrointestinal, gynecological, etc.) according to ICD-10 codes. Patient PD-L1 data was also evaluated for patients depending on metastatic vs primary tumor biopsy, location of biopsy site, and histology. For group-wise analysis, patients in this cohort were grouped into (i) those who received a COVID mRNA vaccination within 100 days before tissue biopsy, (ii) patients who received a COVID mRNA vaccine more than 100 days before tissue biopsy, and (iii) patients who did not receive a COVID mRNA vaccination prior to PD-L1 biopsy.

Healthy Human Subjects: Healthy subject blood was collected and stored as serum and PBMCs at baseline, 6 hours, 24 hours, 7 days, and 14 days after COVID-19 mRNA vaccination (Spikevax, 2023-2024 formulation, Moderna). Frozen serum was thawed and analyzed with NULISA Inflammation Panel 250 by Alamar Biosciences. Statistical analysis was completed with Alamar Biosciences NULISA Analysis Software. Heatmaps and graphs of significantly changed cytokines were generated using GraphPad Prism.

Flow Cytometry: PBMCs were thawed in a 37° C. water bath and immediately transferred into a 50 mL tube containing 9 mL of complete RPMI media (RPMI+10% FBS) in a ratio of 1 part cells to 9 parts media. Cells were centrifuged at 400 g for 10 minutes, and the supernatant was decanted. The cell pellet was resuspended in 1 mL of PBS, and incubated with live/dead marker (1 μL per 1000 μL of cell suspension, containing 1-10 million cells per mL) for 30 minutes at 4° C. After incubation, 3 mL of PBS+2% FBS was added, and the cells were centrifuged again at 400 g for 8 minutes and the supernatant was decanted. Cells were then pre-incubated with 5 μL of Human TruStain FcX™ (BioLegend, cat. 422302) per 100 μL of cell suspension for 5 minutes at room temperature. The cells were then washed with PBS and centrifuged at 400 g for 6 minutes. After decanting the supernatant, 10 μL of Brilliant Buffer was added to each tube, and the mixture was allowed to sit for 5 minutes before addition of antibodies targeting extracellular markers and the cells were incubated for 20 minutes at room temperature in the dark. After incubation, the cells were washed and fixed in the dark for 45 minutes at 4° C. with 500 μL of Fixation solution (BD). Cells were then permeabilized with 2 mL permeabilization buffer. Intracellular stains were added in permeabilization buffer, and the cells were incubated for approximately 40 minutes at room temperature. After incubation, cells were washed with permeabilization buffer and resuspended in 200 μL of PBS+2% FBS. Analysis was then conducted using flow cytometry.

Preclinical Experiments: All murine experiments and procedures were approved by the University of Florida's Institutional Animal Care and Use Committee (IACUC). Naive C57Bl/6 female mice were purchased from The Jackson Laboratory. All mice were implanted subcutaneously with 50,000 B16F0 melanoma cells in the right flank. All mice were vaccinated intramuscularly with 25 ug/dose of RNA-LPA (mRNA fraction). Anti-mouse Interferon alpha receptor (alFNAR1, Bio X Cell, cat. BE0241) and anti-mouse Interluekin-1 receptor (aIL1R, Bio X Cell, BE0256) antibodies were administered intraperitoneally (i.p.) at an initial dose of 500 ug/mouse for the first dose, followed by a maintenance dose of 250 ug/mouse twice a week for the remaining treatment period. Anti-PD-L1 (Bio X Cell, cat. BE0101) checkpoint inhibitor was administered at an initial dose of 500 μg/mouse for the initial dose, followed by a maintenance dose of 250 ug/mouse twice a week for the remaining treatment period. Tumors were measured at a frequency of 3 times a week starting on day 8 until more than 25% of mice reached endpoint. Mice were euthanized upon reaching humane endpoint.

mRNA: BioNTech's published Sars-COV2 Spike mRNA sequence was inserted into a backbone with a T7 promoter and synthesized by Genscript. 5′ Spel sight was removed the sequence to allow for restriction of the Poly-A tail. The T7 promoter was changed to have an AGG initiator sequence via sight directed mutagenesis (NEB, cat. E0554) following the companies recommended protocol. Plasmids were grown in NEB5a competent E. coli and purified using RNeasy Maxi kit (Qiagen, cat. 75162) and sequenced via whole plasmid sequencing by Genewiz. Plasmids were restricted by using 2 units/ug Spel HF (NEB, cat. R3133L) for 2 hours at 37 C followed by DNA precipitation. mRNA was synthesized using mMESSAGE mMACHINE™ T7 mRNA Kit with CleanCap™ Reagent AG (Thermo A57620) with addition of CleanCap® Reagent AG (3′ OMe) (Trilink N-7413-5) and N1-Methylpseudouridine-5′-Triphosphate (Trilink N-1081-10). IVT reaction was performed at 20° C. for 10 hours. DNA was removed according to the kits instructions and RNA was purified using RNeasy maxi kit (Qiagen 75162). RNA was eluted in purified RNAse free water and stored at −80° C. until use.

RNA was checked for quality using an Agilent TapeStation 4150. 100 ug/mL of RNA and RNA ScreenTape ladder (Agilent, 5067-5578) was heated to 72 C for 3 minutes. 100 ng of RNA or 1 uL of ladder was added to 5 uL of RNA ScreenTape Sample Buffer (Agilent, cat. 5067-5577) and mixed at 2000 RPM for 1 minute. Samples were run using an RNA ScreenTape (Agilent, cat. 5067-5576).

Fabrication of Lipid Nanoparticles (LNPs): Prior to complexation, RNA 0.5M citrate buffer (pH 3.75) (Teknova, Custom order) was added to the RNA for a final concentration of 0.1M. ALC-0159 (Avanti 880155P), ALC-0315 (Avanti, 8909000), cholesterol (Sigma C8667) and DSPC (850365P) were reconstituted in 100% ethanol at a ratio of 1.7:47.5:40.8:10. Lipids and RNA were mixed at a 3:1 FRR on a Nanonsemblr™ Ignite™ (Precision NanoSystems, now part of Cytiva) for an N/P ratio of 6. RNA-LNPs were dialyzed overnight at 4 C with two buffer exchanges at 3- and 6-hour time points using a 3.5 kDA dialysis cassette (Thermo, cat. A52967). After dialysis, LNPs were filtered through a 0.2 uM Supor EX ECV filter (Pall KS2ECV2S). LNPs were concentrated using 30 kDA Amicon filters (Millipore UFC903024) by centrifugation at 2000G. Final LNP formulation had 48% sucrose PBS solution added at a 1:3 ratio for a final concentration of 12%.

RNA concentration was determined in LNPs using a RiboGreen assay (Invitrogen, cat. R11490) using a BioTek Cytation 3 plate reader. RNA standard was diluted to 20 μg/mL and used at a range from 2.5-0.1 ug/mL and RNA LNPs were diluted for a final dilution factor of 1000. Encapsulation efficiency was determined by comparing readings to LNPs in TE buffer vs LNPs in 1% triton-X100. LNPs were diluted up to 250 μg/mL with PBS prior to injection. Empty LNPs were used at a volume consistent with the dose of RNA-LNPs fabricated using the same steps and same amounts of lipids. Fluorescence values were plotted in a standard curve with an extrapolation factor set to 1.1. The values from wells with triton X (total mRNA) and TE buffer (free mRNA) was entered into the equation:

EE %=1−Free mRNATotal mRNA EE %=1−Free mRNATotal mRNA

RNA-LNPs were also loaded into a 1% agarose gel made in 1×TAE buffer (company, cat) with GelRed™ (Biotium, Cat #41003-T). 500 ng of RNA loaded into LNPs and free RNA was mixed with 6×SDS (NEB, cat. B7025S) free loading dye and ran on 90V for 45 minutes.

Particle size was determined using the Malvern Panalytical's Zetasizer Ultra and NanoSight's nanoparticle tracking analysis (NTA) equipment. Before analyzing each sample for particle size distribution and PDI using the Zetasizer equipment, LNPs were diluted 100 fold with HyClone HyPure water (Cytiva™, molecular biology grade), mixed 20 times and ran under Zetasizer Ultra (triplicate measurements) at 25° C. with 30 second incubation. Samples were further characterized for surface zeta potential measurements, ran for triplicate measurements and plotted as average mV with standard deviation exported from the ZS explorer software. Additionally, orthogonal particle size distribution and concentration measurements were carried out using Malvern's NanoSight NS300 NTA equipment. Each LNP sample was diluted 500-fold with PBS and then ran through the equipment for five captures using the optimum camera settings. All captures were analyzed at the optimum threshold of detection.

Tissue collection, Plasma from blood: Mice were bled via retroorbital route using heparinized capillaries (FisherScientific, cat.22-362566) collecting a maximum of 200 uL into EDTA coated tubes (FisherScientific, cat.NC9414041) Whole blood was centrifuged at 1200G for 15 minutes. Plasma was carefully collected and stored at −80° C. until use.

Tissue collection, Processing of tumors for flow cytometry: Tumors were carefully dissected from euthanized mice with the external fibrous sac left intact. Tumors were bisected using a scalpel and half of each sample was placed into a GentleMacs column GentleMACS C Tube (Cat #130-096-334 with) with an enzyme mix containing 10 mg/ml of Collagenase (Sigma-Aldrich, Cat #C5138-1G), 1 mg/ml of Hyaluronidase (STEMCELL Technologies, Cat #07461), and 200 mg/ml of DNase (Sigma-Aldrich, Cat #D5025-150KU). Samples were run on (cycle name and parameters) cycle and centrifuged at 500G for 5 minutes after completion. Pellets were resuspended in cold PBS and filtered through a 70 μm cell strainer. Samples were washed twice using cold PBS and then manually counted using a hemocytometer.

Tissue collection, Isolation of splenocytes: Whole spleens were collected from euthanized mice and placed in cold RPMI medium. Spleens were then teased through a 70 um filter and then lysed for 5 minutes at 37 C using 1×BD Pharmalyse buffer (BD, Cat #555899). Lysis buffer was quenched with media and centrifuged at 500G for 5 minutes. Splenocytes were resuspended in cold PBS and filtered through a 70 μm cell strainer and washed 1× with PBS prior to being counted using a Beckman Coulter Vi-cell XR.

Flow Cytometry: 1×10⁶ cells from both tumors and spleen were placed into 96 well v-bottom plates. Unless stated otherwise, wash steps were done by centrifugation steps were performed at 500 G for 5 minutes at 4 C and resuspension of cells with 200 uL of buffer with mixing done via pipetting. Cells were washed with cold PBS and stained with 100 uL of live/dead stain (Thermo, cat. L10119) for 30 minutes at 4 C. Live dead dye was quenched with 100 uL of cold PBS and washed one time with cold FACs buffer (PBS with 2% FBS). Cells were centrifuged and resuspended with 10 uL True stain FCX buffer (BioLegend, cat. 422302) diluted to 100 ug/mL with FACs buffer for 10 minutes. 90 uL of antibodies (Antibody table 9 and 10) and Brilliant Stain buffer (BD, cat. 563794) were added and incubated for 30 minutes at 4° C. 100 uL of FACs buffer was added to each well and washed two times with cold FACs buffer. Cells were fixed for 15 minutes with 100 uL of Cytofix buffer (BD, cat. 554655) at 4° C. 100 uL of PBS was added to each sample and cells were washed 2 times and stored in FACs buffer at 4° C. in the dark until analysis. Compensation was performed using Ultracomp eBeads (Thermo Fisher Scientific, cat. 01-2222-42) and ArC amine reactive compensation beads (Thermo Fisher Scientific, cat. A10346). Results were acquired using a BD Symphony A3 and analyzed using FlowJo v10.10.0.

Activation-induced marker (AIM) assay: Aim Assay was performed as previously described. 13 In this iteration, whole splenocytes were used rather than isolated T cells. In brief, 24 hours after the last RNA-LPA vaccine, splenocytes were collected for antigen recall assay. Spleens were collected and processed as described above. 1 million splenocytes were cultured in a 96 well round bottom plate in T cell medium containing RPMI 1640 (Gibco, cat. 11-875-119), 10% fetal bovine serum (ThermoFisher Scientific, cat. 35-011-CV), 1% penicillin/streptomycin (Gibco, cat. 30-002-CI), 1% MEM non-essential amino acids (NEAA, Gibco, cat. 11140050), 1% sodium pyruvate (Gibco, cat. 11360070), 0.1% beta-mercaptoethanol (BME, Gibco, cat. 21985-023) and 0.2 ug tumor associated peptides without cytokines. Peptides were chosen based on their suggested upregulation in melanoma tumors. 13 Selected peptides were purchased from JPT Peptide Technologies (Germany) and reconstituted according to manufacturer guidelines. Co-culture was maintained for 48 hours in an incubator at 37° C. and 5% CO₂. Next, cells were collected and stained for activation-induced marker (AIM) assay for evaluation of activation of antigen-specific T cells. Cells were centrifuged and resuspended in FACS buffer containing antibodies mix (Antibody table 11). Cells were washed three times and fixed using fixation medium (Thermo. cat. GAS001S100) for 15 minutes at room temperature. Cells were washed 1 time with PBS and were stored in FACs buffer at 4 C in the dark until analysis. Results were acquired using a BD Fortessa Ill and analyzed using FlowJo v10.10.0.

ELISA and multiplex analysis: Plasma was analyzed for cytokine concentration using ELISAs and multiplex cytokine arrays. A portion of the plasma samples were assessed for IFN-α through ELISA (Invitrogen, cat. BMS6027). Plasma was also sent for analysis by Eve Technologies for multiplex cytokine array (MD44).

For quantification of spike IgG, mice that received three vaccines/vehicle doses during tumor studies were bled once humane endpoint was reached. Spike specific IgG was determined using an Anti-mouse Sars-CoV-2 IgG titer assay (AcroBiosystems, cat. RAS-T023). Plasma was diluted 100,000-fold and ran according to the company recommended parameters for semi-quantitative analysis.

Immunofluorescence: Half of bisected tumors were fixed with 4% paraformaldehyde at 4° C. overnight. Samples were washed three times using PBS and then immersed successively into 10%, 20% and 30% sucrose for cryopreservation. Tissue was then embedded in O.C.T. (Tissue-Tek, Cat #4583) and stored at −80° C. Blocks of tissue were moved to −20° C. for 24 hours and were sectioned at 30 μm thickness using a Leica cryostat, and the sections were placed onto microscope slides. Prior to staining, slides were brought up to room temperature for 15 minutes and washed three times with PBS. Tissue was blocked at room temperature for 1 hour using a blocking buffer containing 2% goat or donkey serum, 1% bovine serum albumin, and 0.1% Triton X-100 in 1×PBS. Sections were then stained with primary antibodies (Antibody Table 12) in blocking buffer overnight at 4 C. Following incubation, sections were washed three times with PBS and stained with secondary antibodies (Antibody Table 12) diluted in blocking buffer for 1 hour at room temperature. After three 5 minute PBS washes, sections were incubated with DAPI (1:1000 in PBS) for 10 minutes at room temperature. Following a triple PBS wash, sections were mounted with Prolong Glass Antifade Mountant (Thermo Fisher Scientific, cat. P36984) and covered with a cover glass. Images were acquired using a Leica Stellaris 8 WLL Spectral Confocal Microscope. Image processing was performed with Fiji-ImageJ software (NIH) and Imaris.

### Results

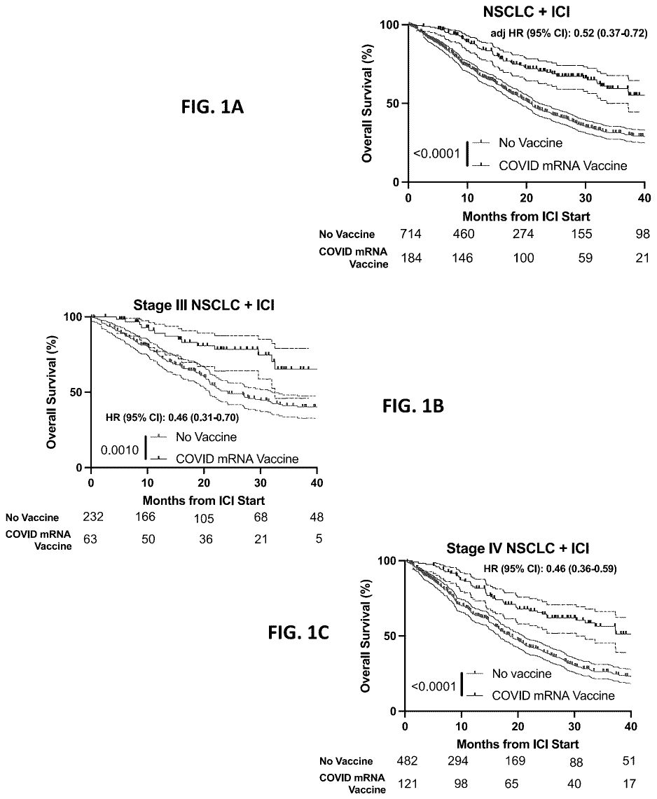

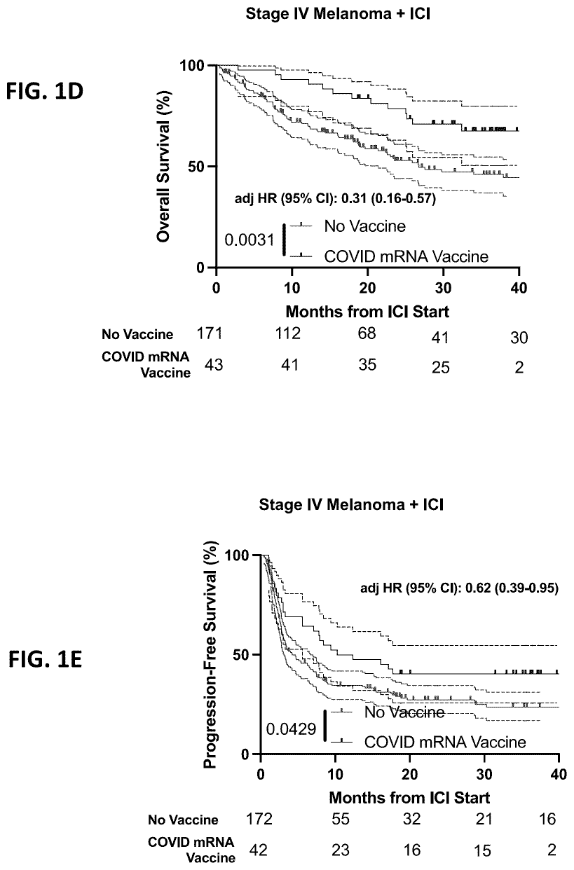

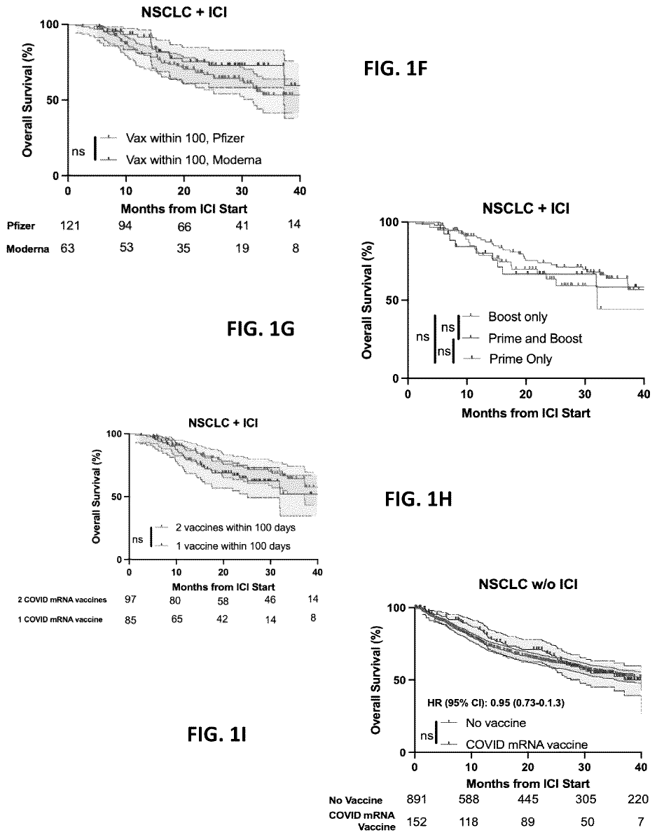

COVID mRNA vaccines are associated with improved survival outcomes: To determine whether COVID mRNA vaccines were associated with improved responses to immune checkpoint blockade in patients, we first compared overall survival among a cohort of patients with Stage III/IV non-small cell lung cancer (NSCLC) treated at MD Anderson Cancer Center (MADCC) between August 2019 and August 2023 (n=2406) (Table 1). After controlling for 34 covariates including performance status, clinical stage, steroid use, tumor proportion score (TPS) of PDL1, disease burden at presentation, cycle of therapy, mutation status, concurrent therapies, and comorbidities with multivariate Cox-proportional hazards regression (Tables 2-3), we found that receipt of a COVID mRNA vaccine within 100 days of initiation of treatment with ICI was associated with a doubling of median OS (20.7 months vs 42.43 months) and a doubling of three year overall survival (OS) (31.0% vs 59.5%, adj HR 0.52, 95% Cl 0.37-0.72, p<0.0001) (FIG. 1A). This survival advantage was similar for: patients with Stage III (HR 0.46, 95% Cl 0.31-0.70) (FIG. 1B) and Stage IV NSCLC (HR 0.46, 95% Cl 0.36-0.59) (FIG. 1C); patients who received mRNA vaccines from either vaccine manufacturer (FIG. 1F); and patients who had or had not received a previous COVID mRNA vaccine (FIG. 1G). In addition, patients who received two vaccines in the 100 days surrounding initiation of ICI experienced numerically improved but statistically insignificant improvements in overall survival (FIG. 1H). In contrast, patients who received COVID mRNA vaccines but did not receive ICI experienced no survival benefit, suggesting that COVID mRNA vaccines require ICI for antitumor efficacy (FIG. 1I).

We then repeated this analysis in a separate cohort of patients treated with front-line ICI for metastatic melanoma. After accounting for 26 covariates with Cox Proportional Hazards Regression (Tables 4-8), we found that receipt of a COVID mRNA vaccine within 100 days of initiating ICI was associated with substantially improved OS (median OS 27 months vs unmet, 36-month OS 46% vs 68%, adj HR 0.31, 95% Cl 0.16-0.57, p=0.003) and progression free survival (PFS) (median PFS 4 months vs 12 months, 36-month OS 23% vs 41%, adj HR 0.62, 95% Cl 0.39-0.95, p=0.043) (FIG. 1D-E).

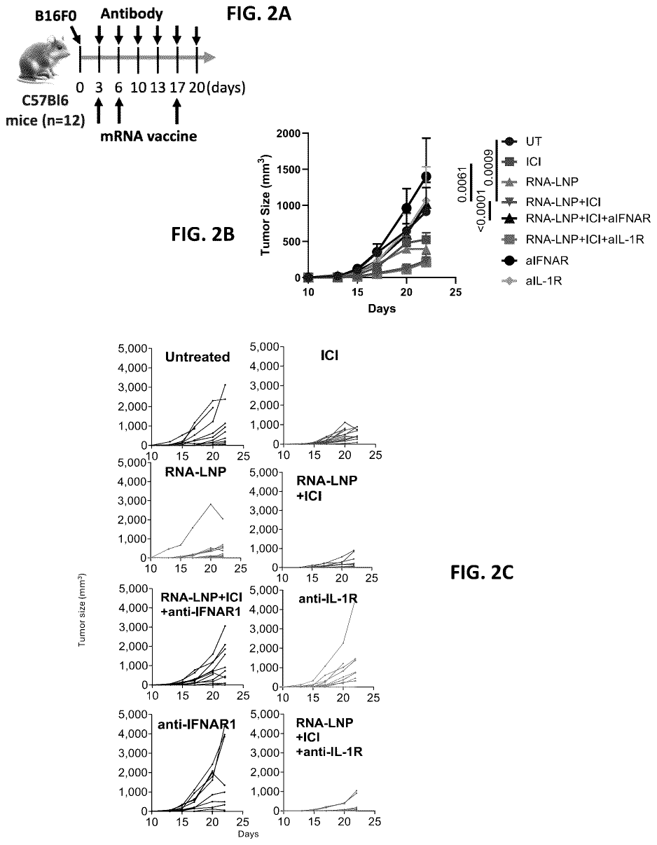

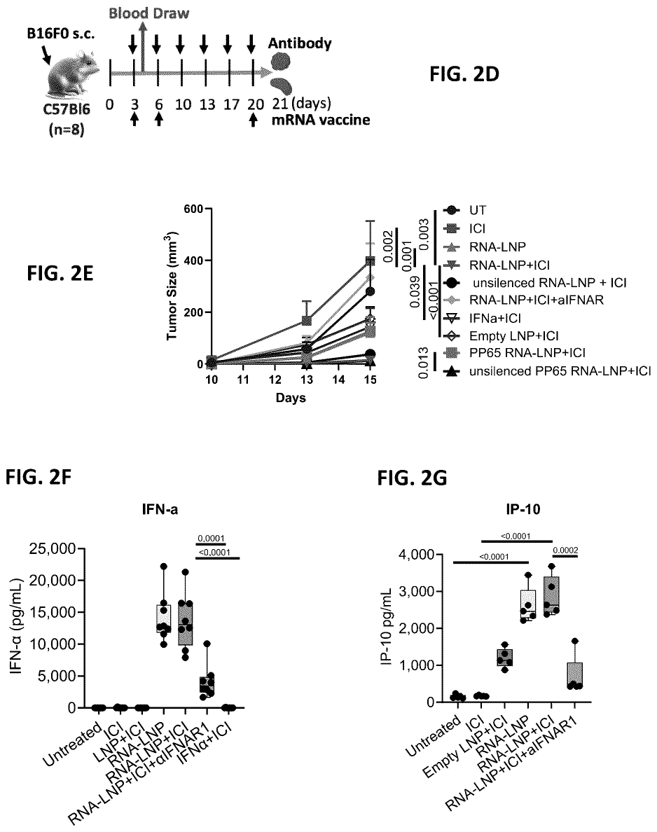

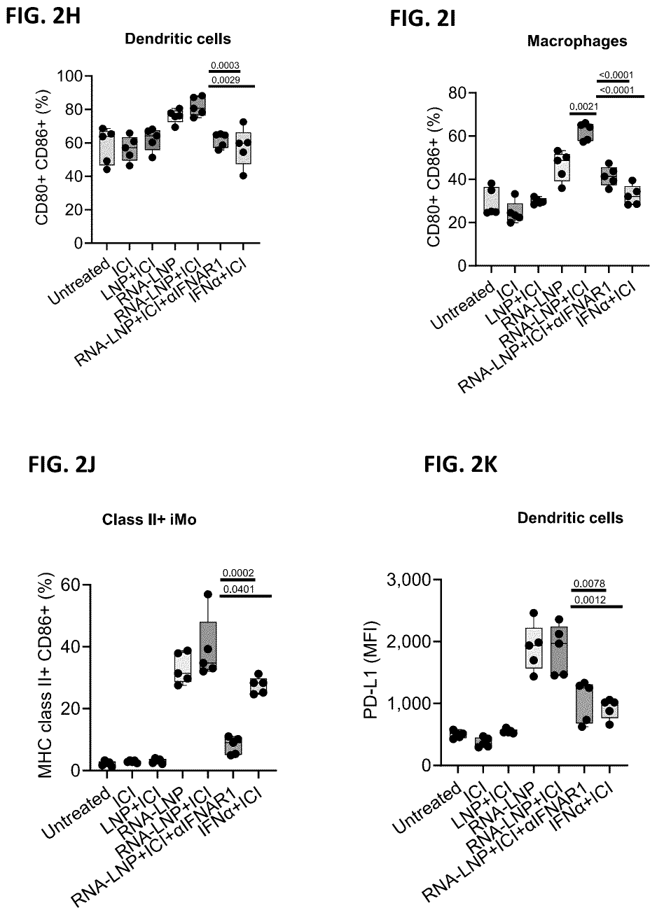

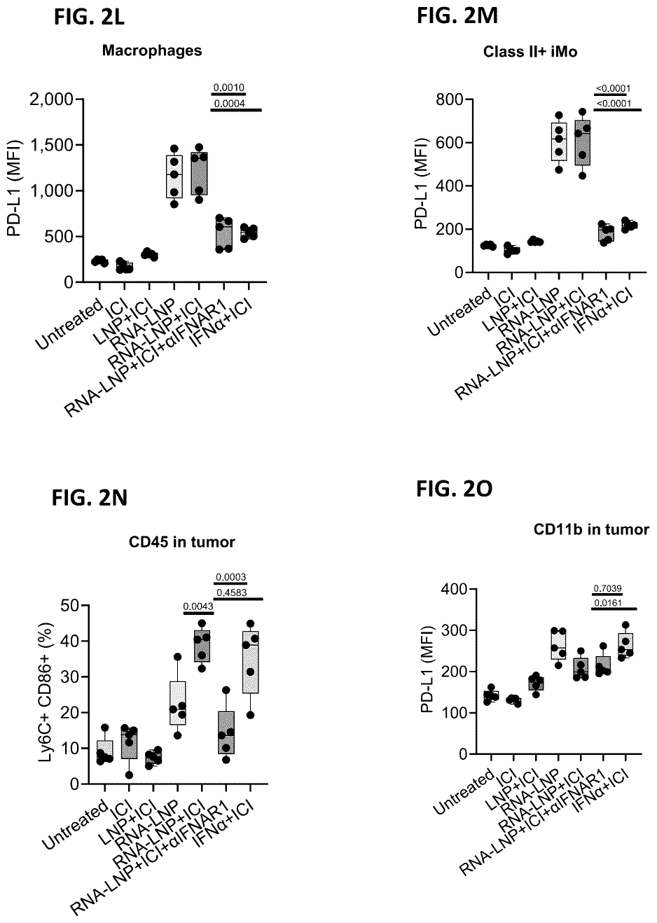

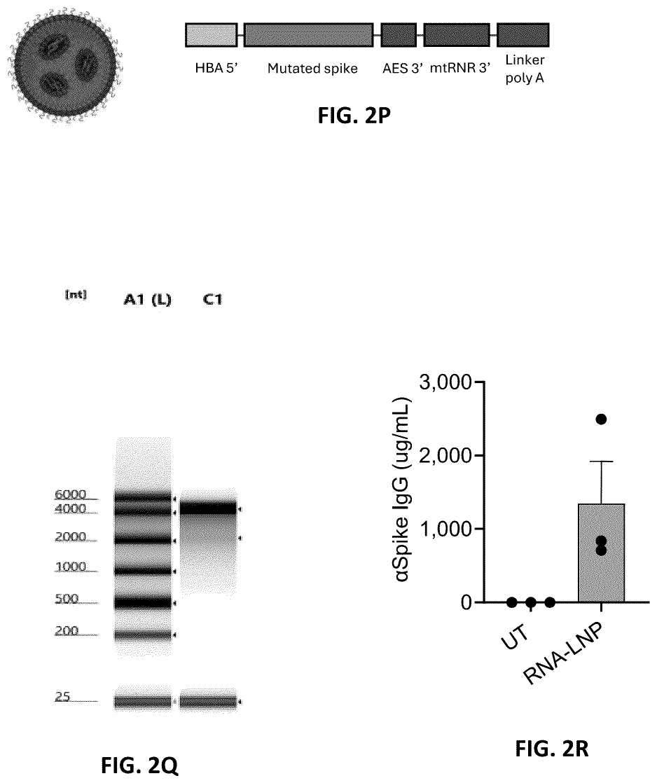

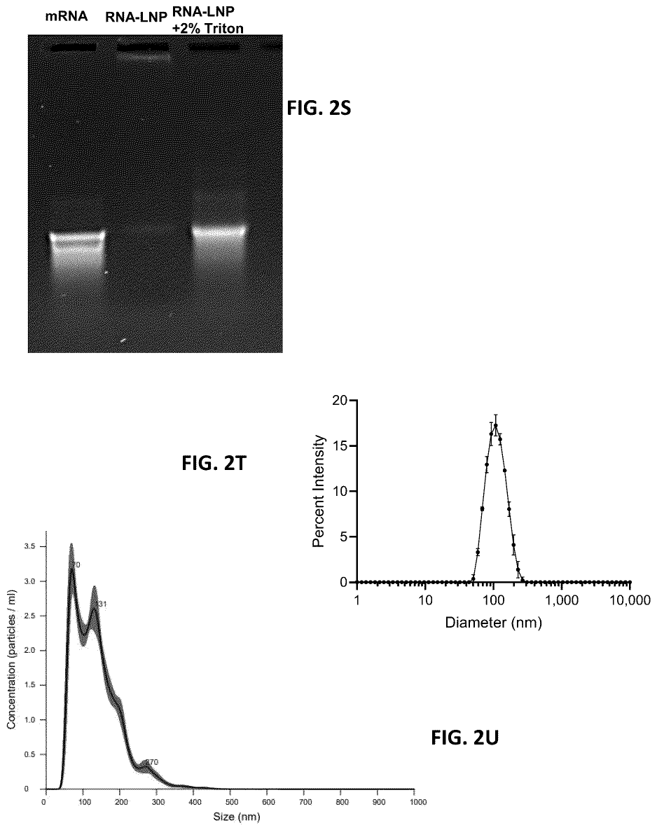

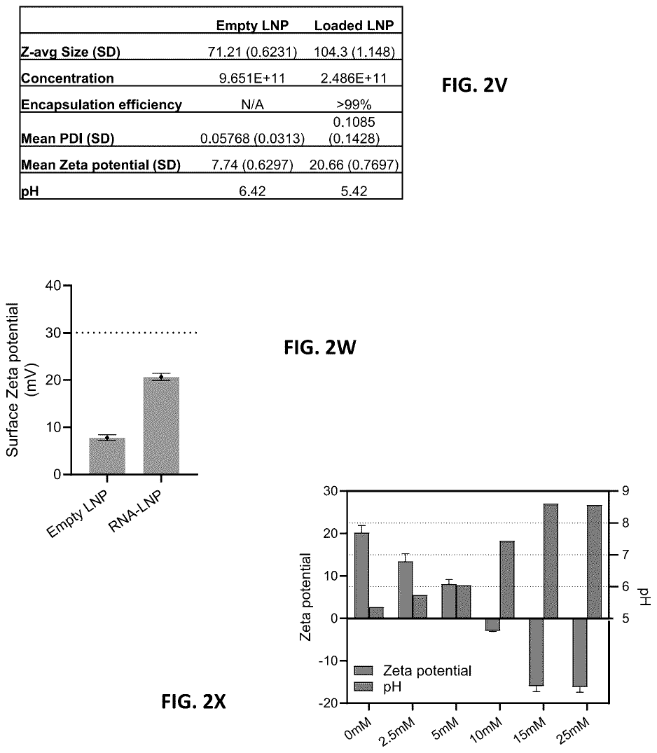

Improved survival with COVID mRNA vaccination is dependent on type 1 interferon: To demonstrate whether effects observed in humans could be modeled in animals, we recreated formulations in commercial preparations for administration to tumor bearing animals in conjunction with ICI. Since SARS-CoV-2 spike protein has been fully sequenced, an mRNA construct was synthesized from the original Wuhan strain and validated fidelity of synthesis based on mRNA size (FIGS. 2P-2Q) and ability to elicit neutralizing antibodies following in vivo administration to animals (FIG. 2R). Administrations were formulated in lipid-nanoparticles (LNPs) with 100% efficiency (FIG. 2S) and met specifications delineated in COVID-19 mRNA vaccine preparations for size distribution, polydispersity and charge (FIG. 2T-2X), the latter of which we found could be disproportionately affected by buffer conditions leading to net positive charge (FIG. 2X). Murine B16F0 melanoma was chosen as a model to test immunogenicity and efficacy of spike RNA-LNP vaccines due to effects of the vaccine in the clinical setting and because B16F0 is a poorly ICI responsive tumor model. Tumor bearing animals were administered three vaccine doses in conjunction with ICI treatment alone or in combination with cytokine-blocking antibodies (FIG. 2A). While RNA-LNPs and PD-L1 blockade provided no benefit as single agents, combination therapy with RNA-LNPs and PD-L1 blockade strongly inhibited tumor growth (FIG. 2B-2C). Although we previously demonstrated a role for IFN-α in the response to mRNA vaccines, recent evidence suggests a dominant role for IL-1 signaling in responses to the specific RNA-LNPs targeting the COVID Spike protein.¹⁴ The importance of each pathway was evaluated with antibodies blocking the IL-1 and IFN-α receptors. While blockade of IL-1R had no effect on tumor growth, anti-tumor responses were completely abrogated with pre-administration of type I interferon signaling with IFNAR1 monoclonal antibodies (FIG. 2B-2C). These results highlight the importance of interferon in driving innate and adaptive immunity, and the ability to manipulate and reset the immune setpoint away from tolerance toward effector immune responses in the presence of ICI.

Antitumor effects of COVID mRNA vaccines are driven by innate immune sensing of mRNA: Next, the importance of mRNA species included in the vaccine was assessed (FIG. 2D-2E). The protein encoded by the mRNA was modified by replacing the Spike mRNA with mRNA encoding the cytomegalovirus antigen pp65, which is overexpressed in glioma but not expressed in B16F0. No significant difference in antitumor activity was observed between these two groups, suggesting that innate immune sensing of the mRNA itself, not the protein encoded by the mRNA, is the primary driver of antitumor activity from mRNA vaccines (FIG. 2E).

Since N1-methyl-pseudouridine is used in COVID mRNA vaccines to “silence” innate immune activation, we hypothesized that “unmodified” mRNA with fully intact motifs to activate pattern recognition receptors would provide even more robust innate immune activation and antitumor activity. Replacing “silenced” pp65 mRNA with “unsilenced” pp65 mRNA resulted in further synergy with ICI, again suggesting that innate sensing of mRNA is a critical driver of antitumor responses (FIG. 2E). Although it was demonstrated that blockade of IFN-α abrogated the antitumor effects of RNA-LNPs, exogenous IFN-α was insufficient for synergy with ICI (FIG. 2E).

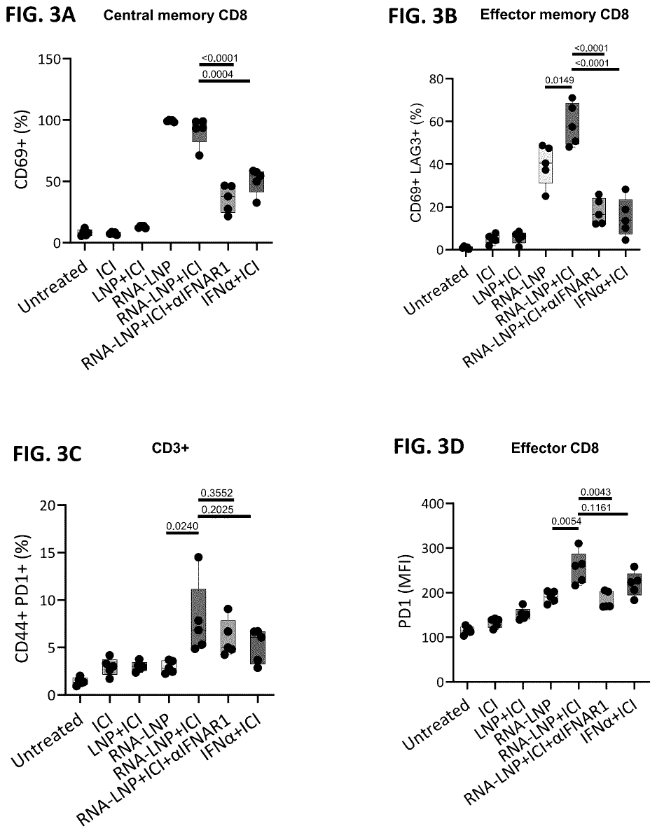

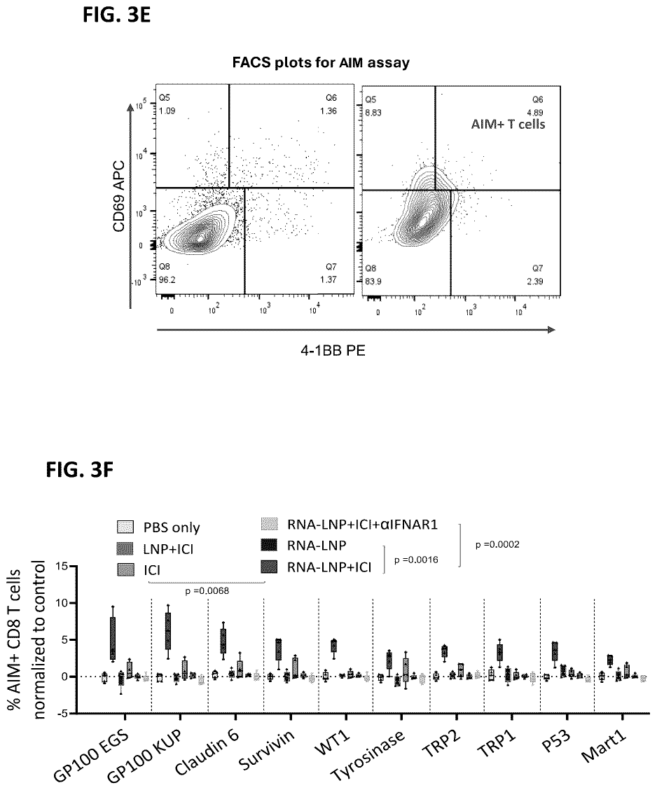

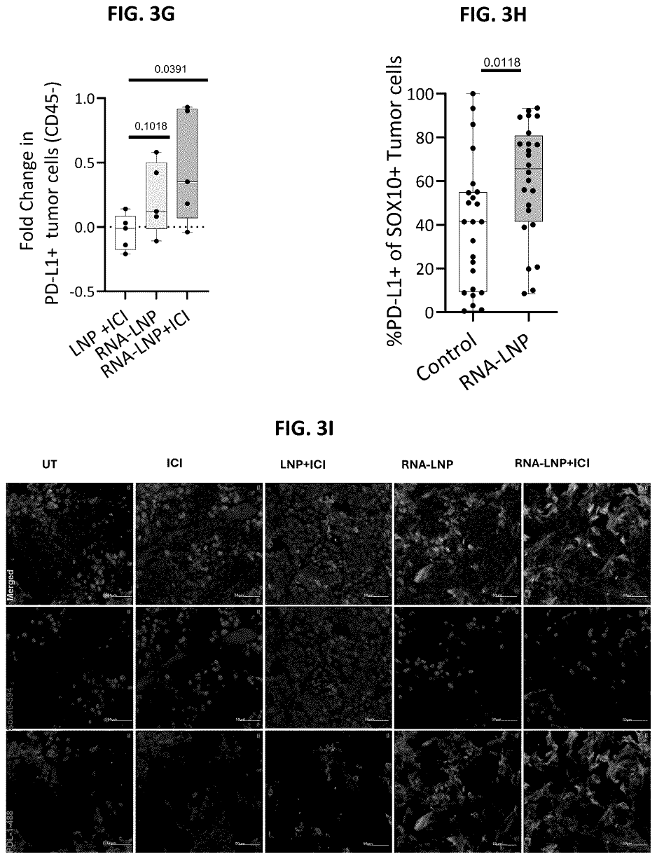

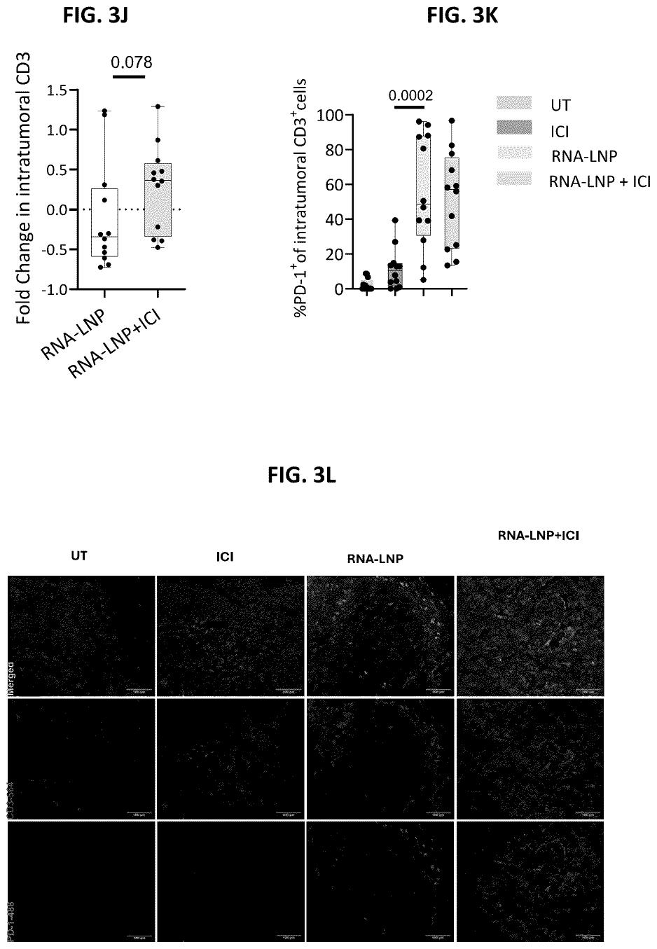

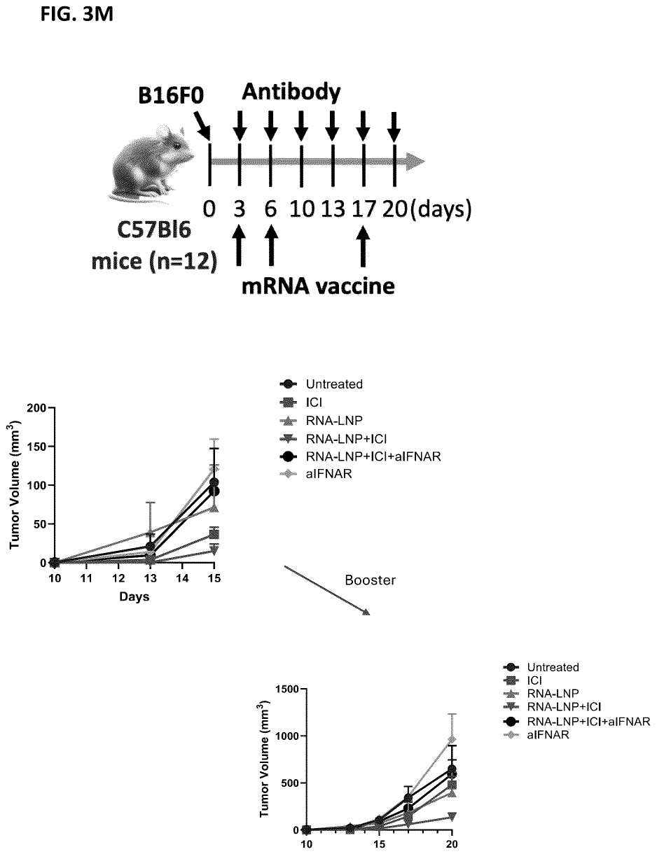

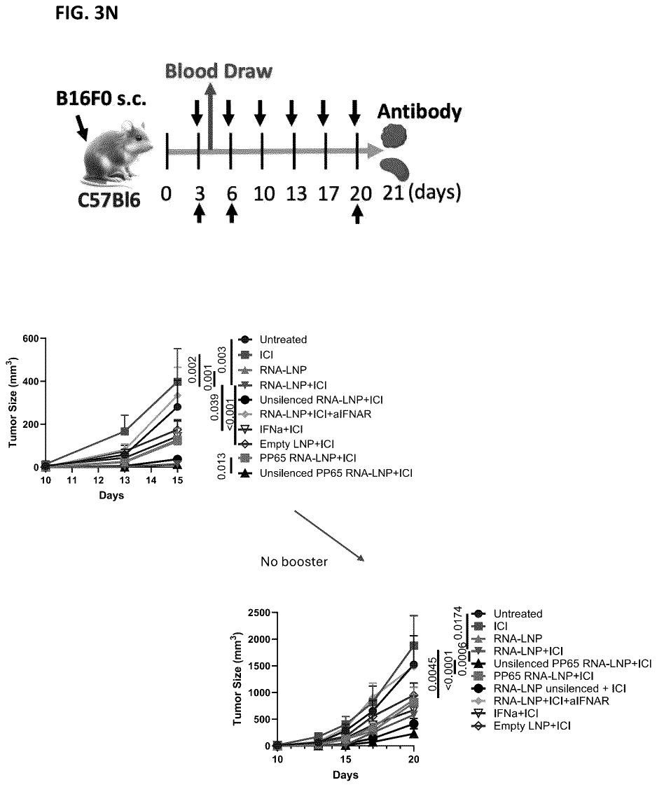

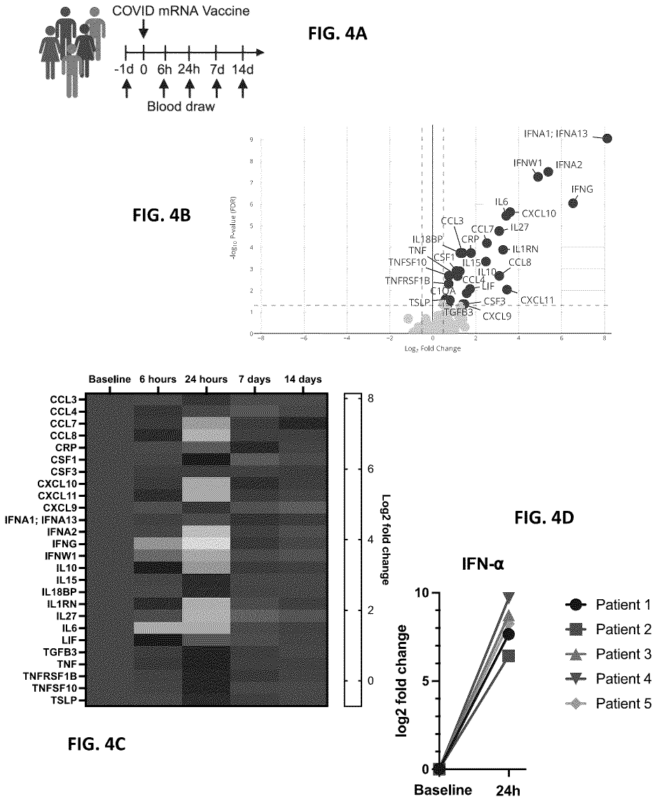

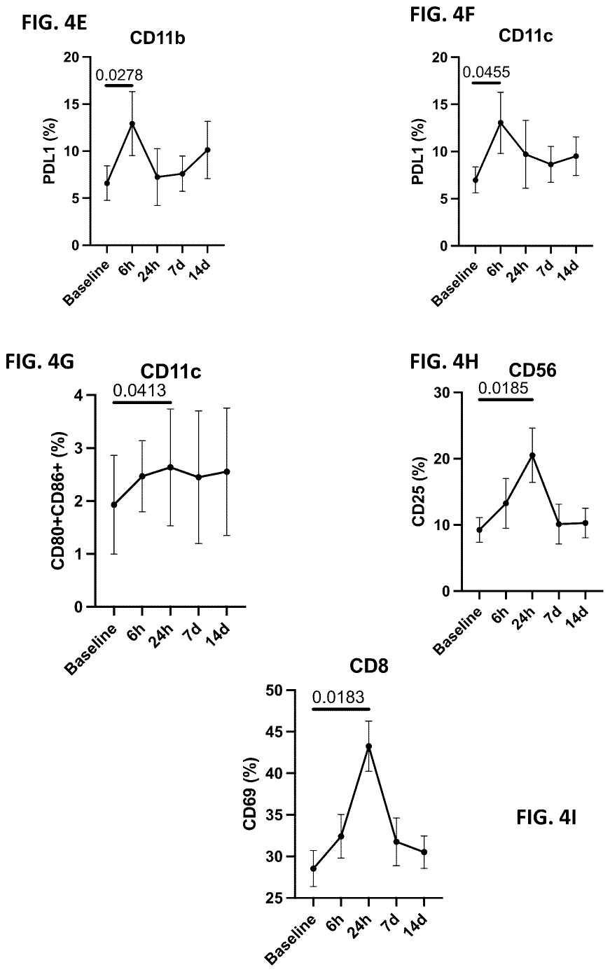

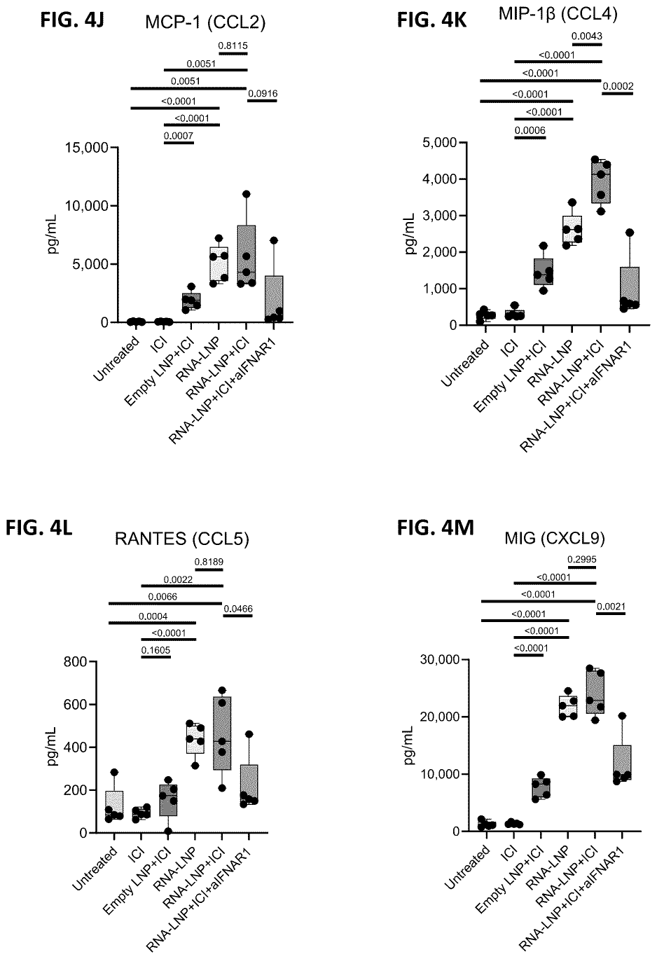

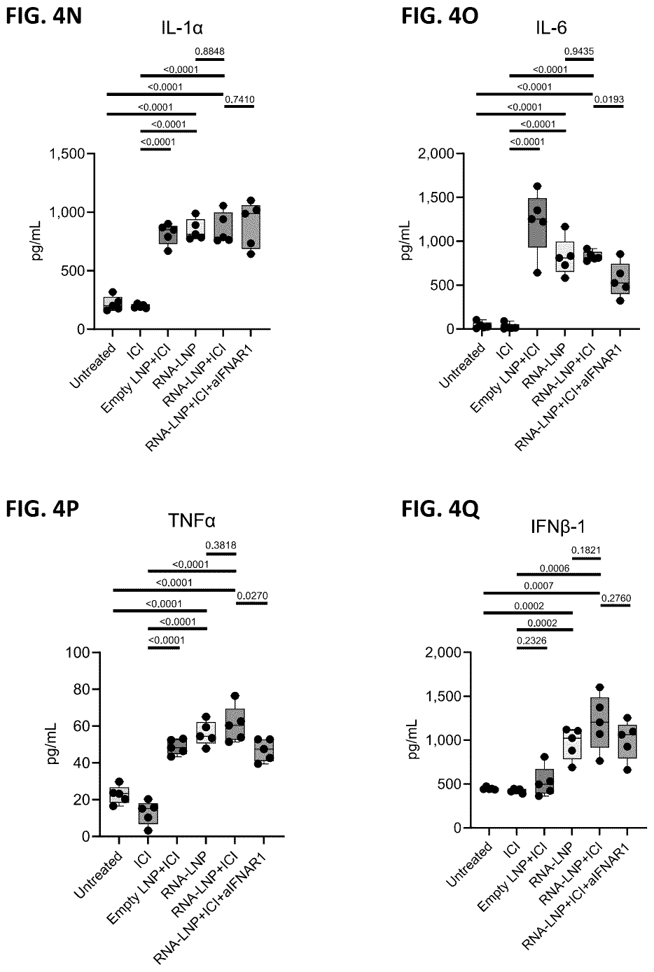

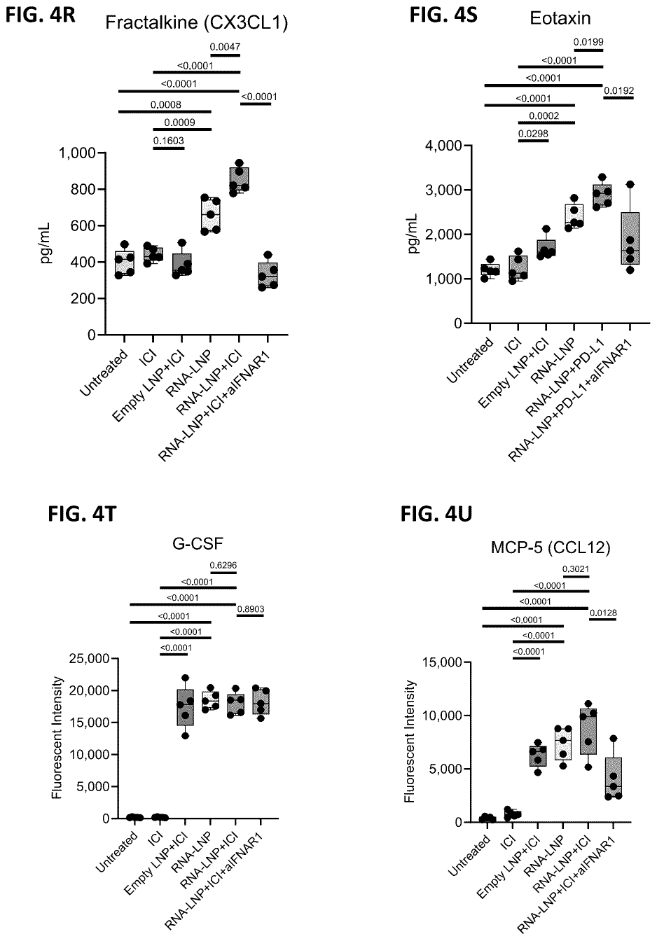

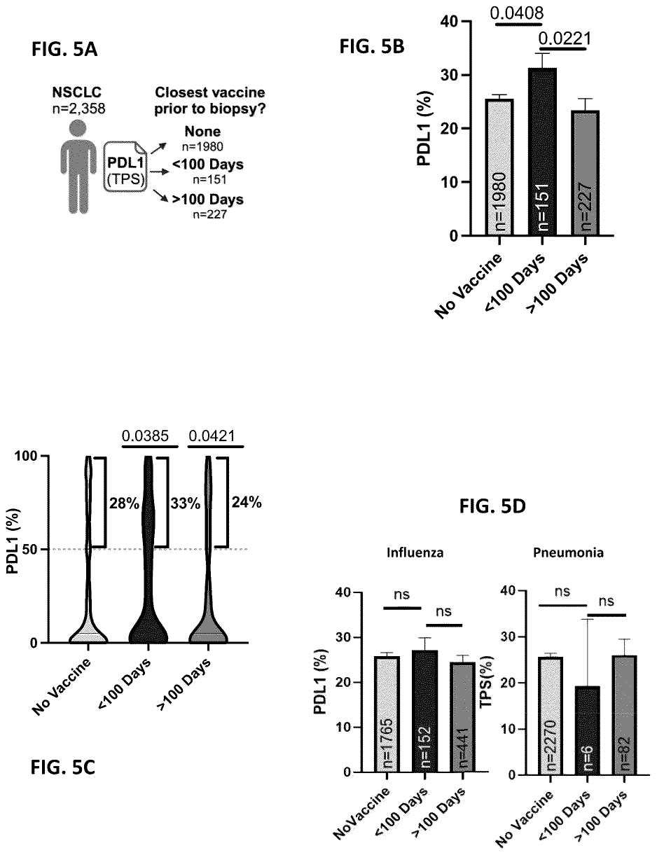

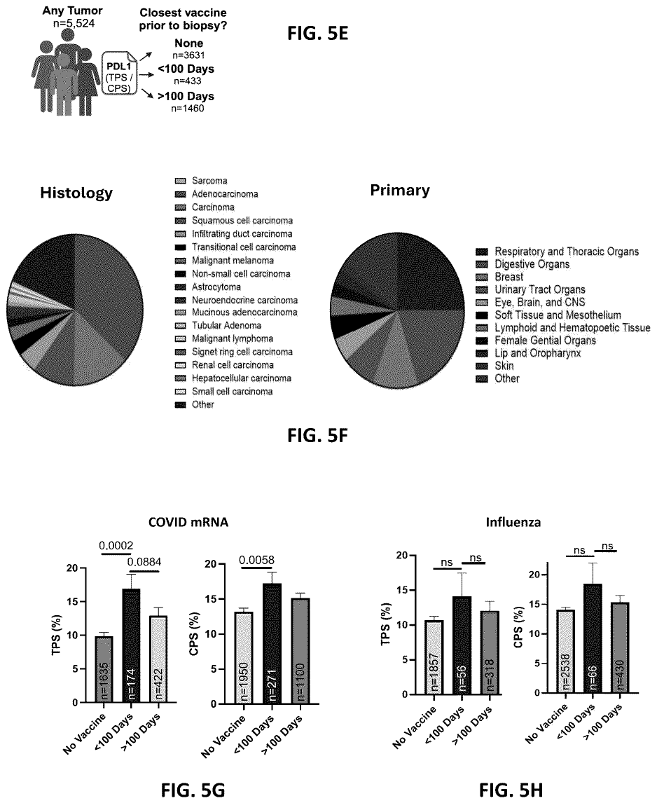

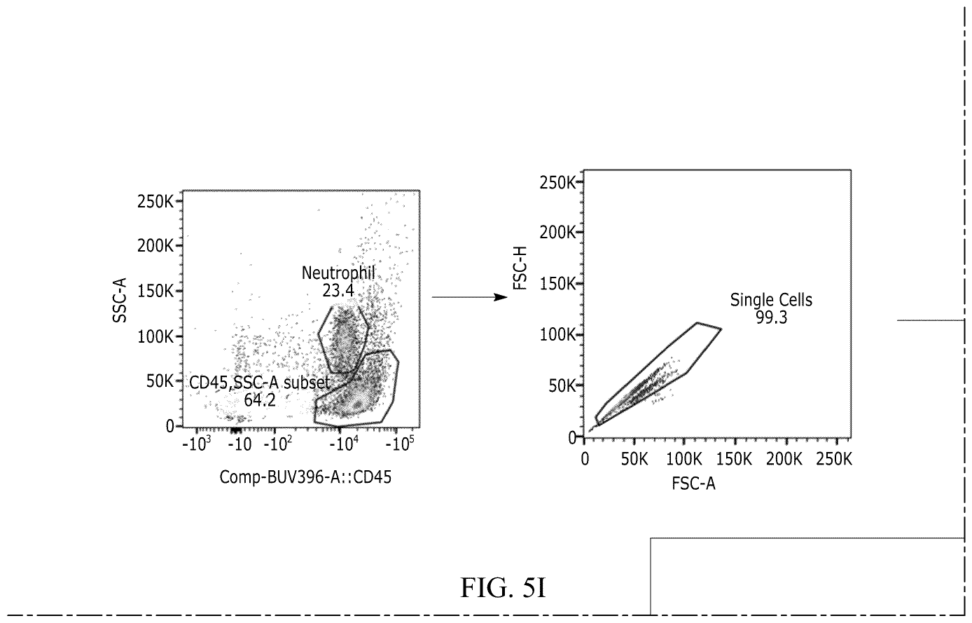

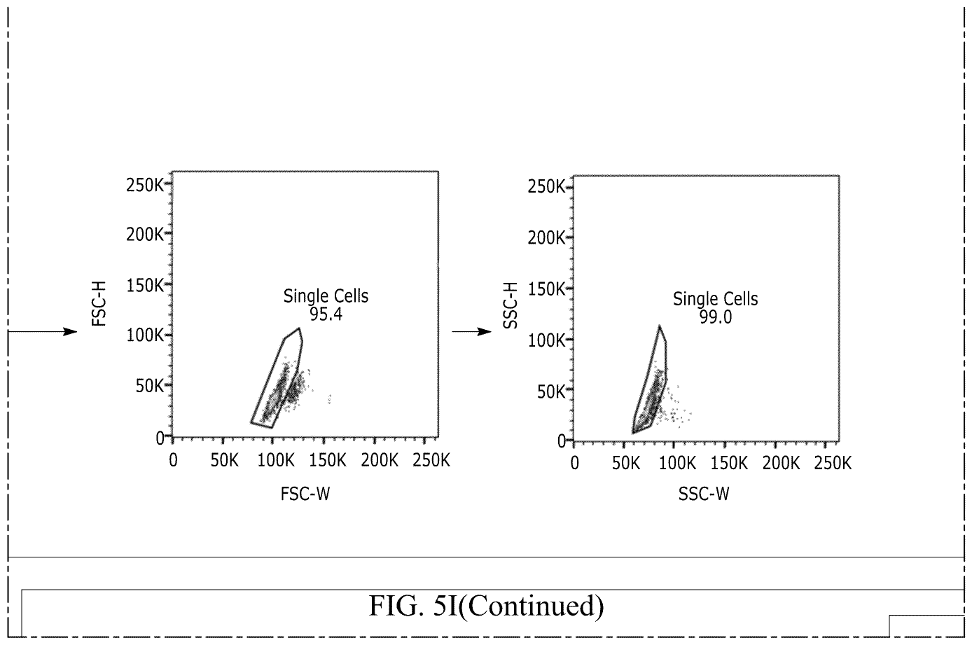

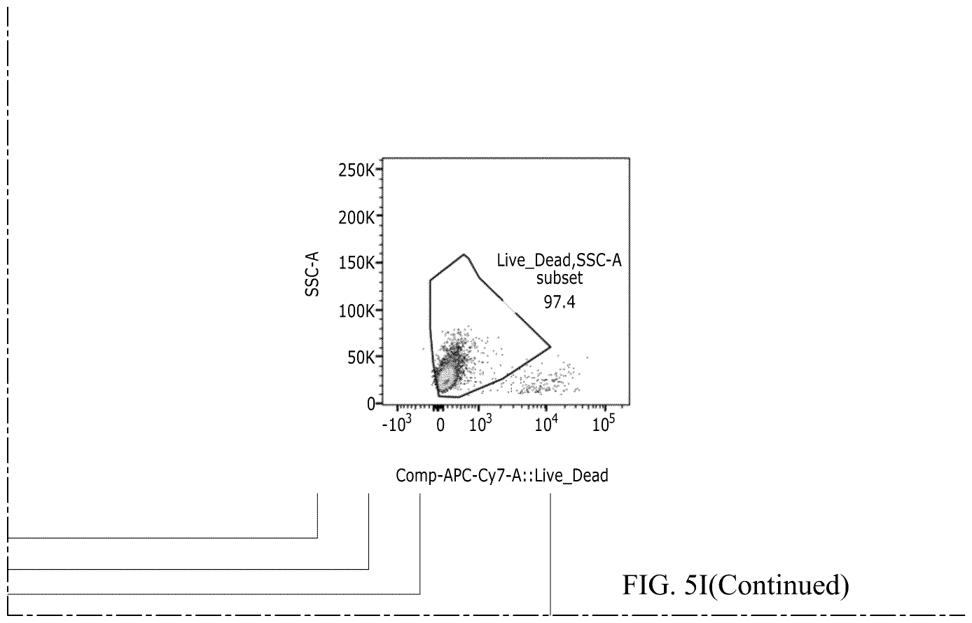

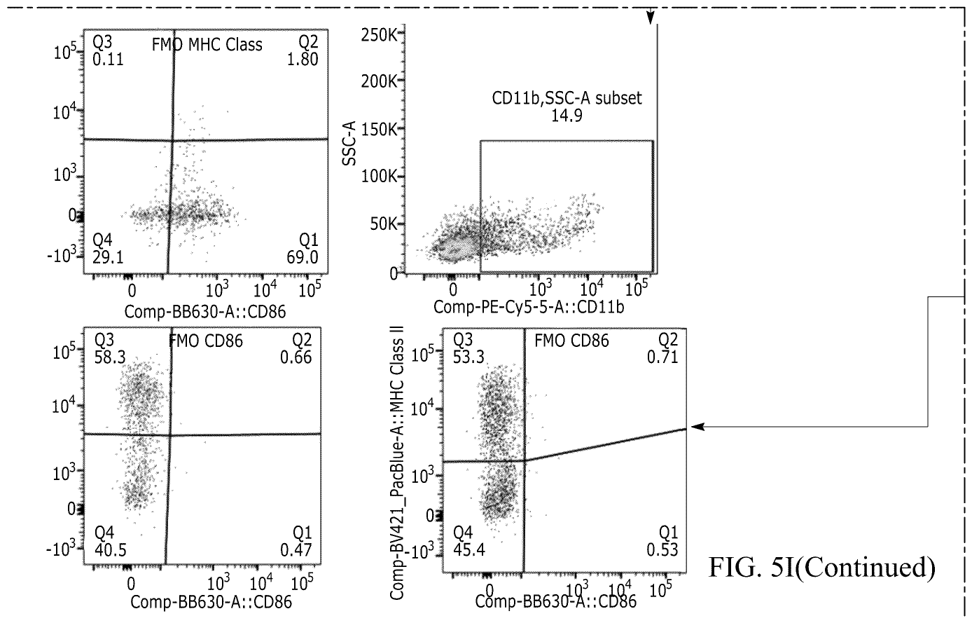

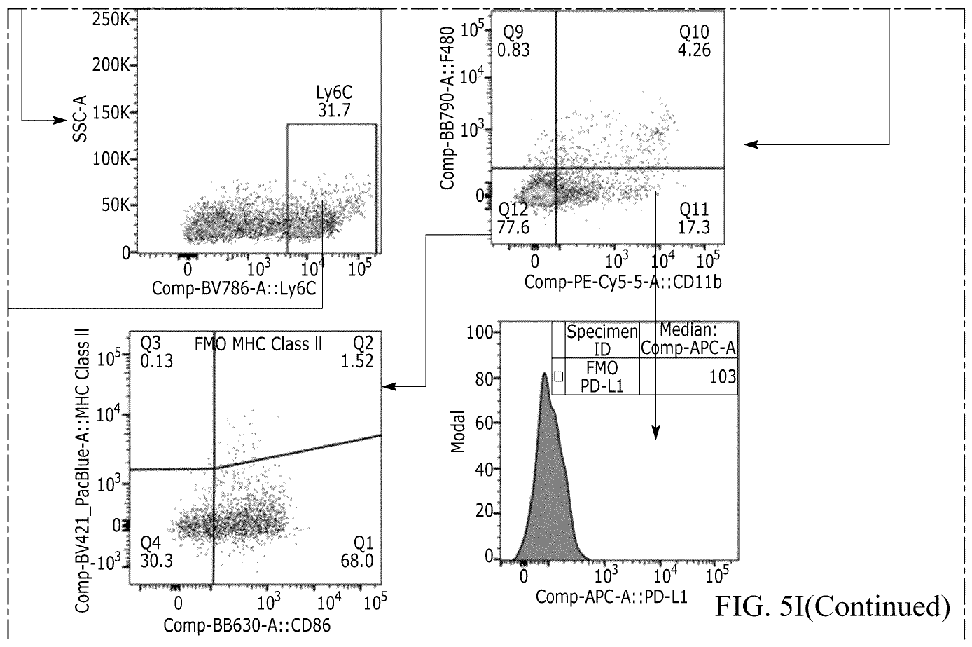

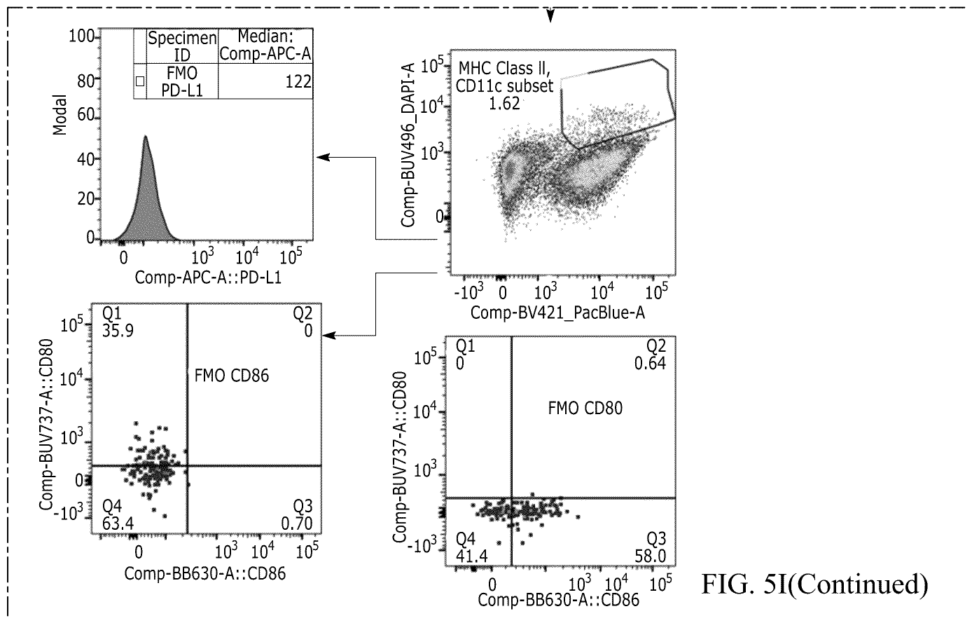

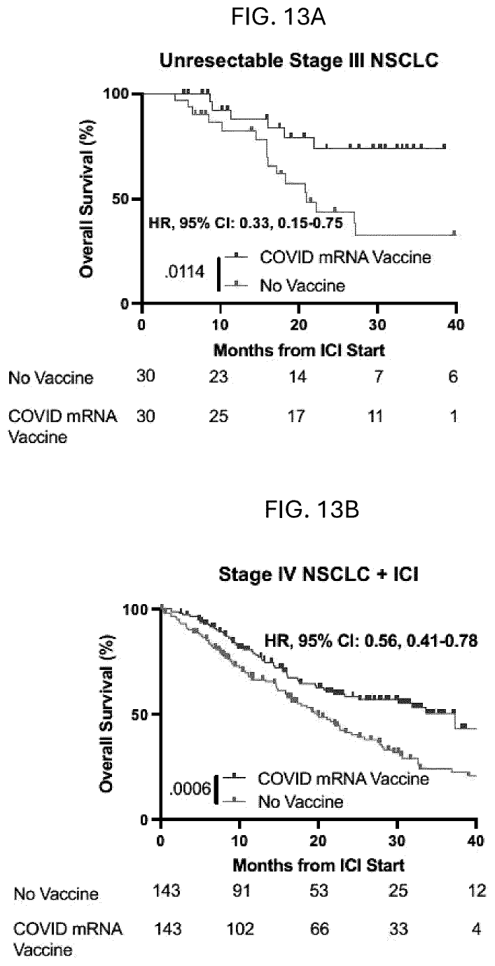

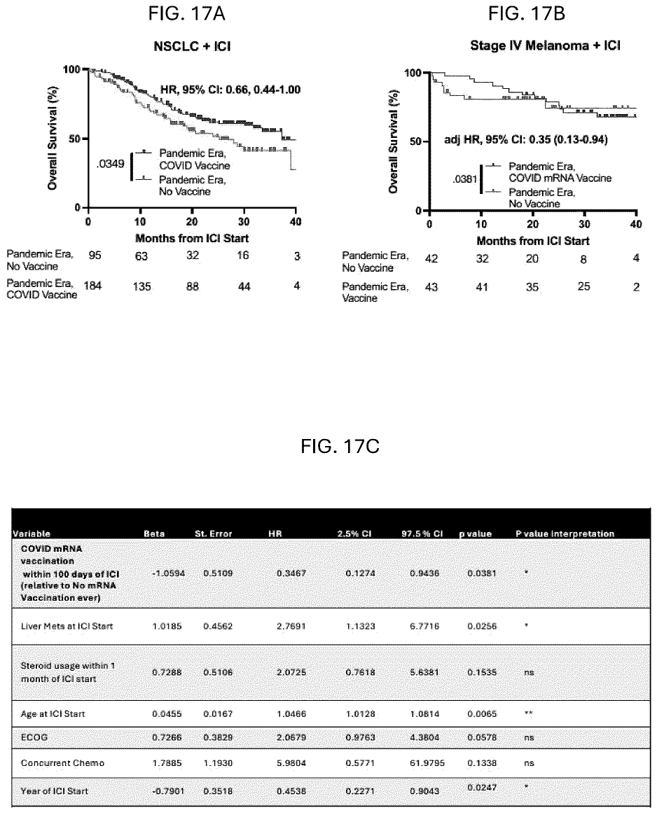

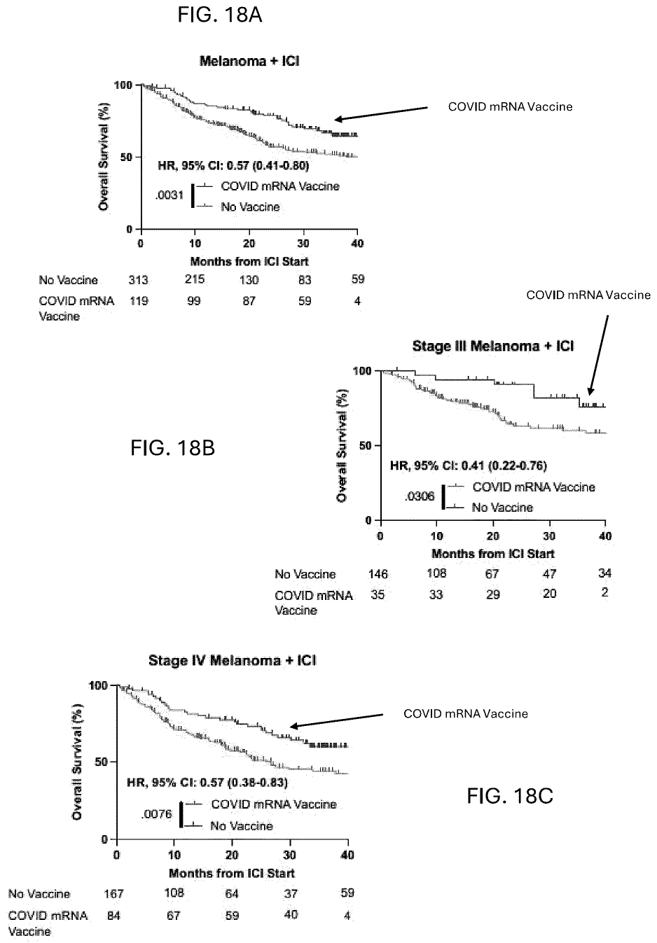

COVID mRNA vaccines reprogram innate immune cells: The impact of RNA-LNPs on innate immune cells was further examined. To allow growth of tumors in all treatment groups, the second “booster” vaccine was delayed to Day 20, and tumors and spleens were harvested from vaccinated animals on Day 21 (FIG. 2D, FIGS. 3M-3N). IFN-α was dramatically elevated in both RNA-LNP and combination groups in conjunction with other Th1 chemokines (FIG. 2F, FIGS. 4J-4Y). This surge in cytokine/chemokine response correlated with an increase in myeloid cell recruitment and activation in the spleens of animals treated with RNA-LNP alone or in combination with ICI. Following spike encoding RNA-LNP, there was precipitous activation and trafficking of antigen presenting cells (APCs) such as dendritic cells, macrophages and inflammatory monocytes expressing MHC class-II (FIG. 2G-2I, FIG. 5I) to lymphoreticular organs in an IFN-α-receptor (IFNAR1) dependent manner. While this response was dependent on interferon signaling, the magnitude of the response could not be achieved by simply administering systemic IFN-α in combination with ICI (FIG. 2G-21). In tandem, myeloid activation extended to the tumor, where receipt of RNA-LNP vaccines was associated with IFNAR1 dependent increases in activated Ly6C+ monocytes (FIG. 2M) and CD11b+ myeloid cells (FIG. 2N). Interestingly, combination therapy with ICIs increases CD45+ cells but decreases CD11b cells suggesting demonstrative of a shift in intratumoral phenotype from immunosuppressive to pro-inflammatory.

COVID mRNA vaccines reprogram adaptive immunity: In addition to the precipitous activation of APCs, spike encoding RNA-LNPs also elicited massive activation and expansion of effector and central memory CD8 T lymphocytes (FIGS. 3A-3B), highlighting the ability of these vaccines to rapidly prime the adaptive immune response and boost memory cell subsets. While RNA-LNPs alone were sufficient for T cell activation characterized by co-expression of CD69, increased expression of PD-1 was only observed with the combination of RNA-LNPs and ICI. Overall, these results illustrate the importance of systemic immunomodulation necessary for recruitment of myeloid cells into lymphoreticular organs and the TME necessary for enhancing likelihood of tumor antigen presentation to cognate T cells. Once activated, T cells can penetrate TME but only survive and persist in the presence of ICIs.

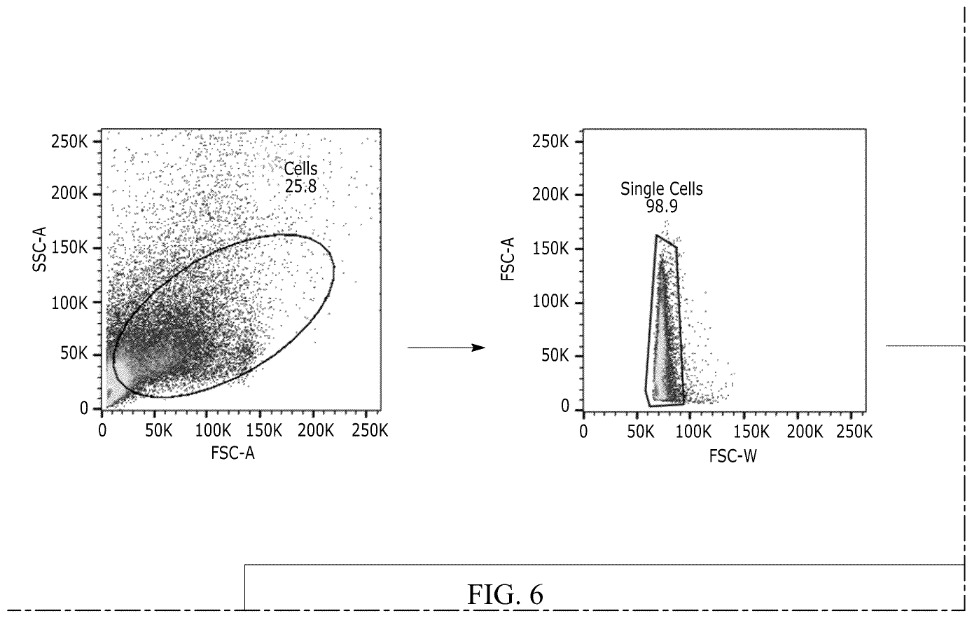

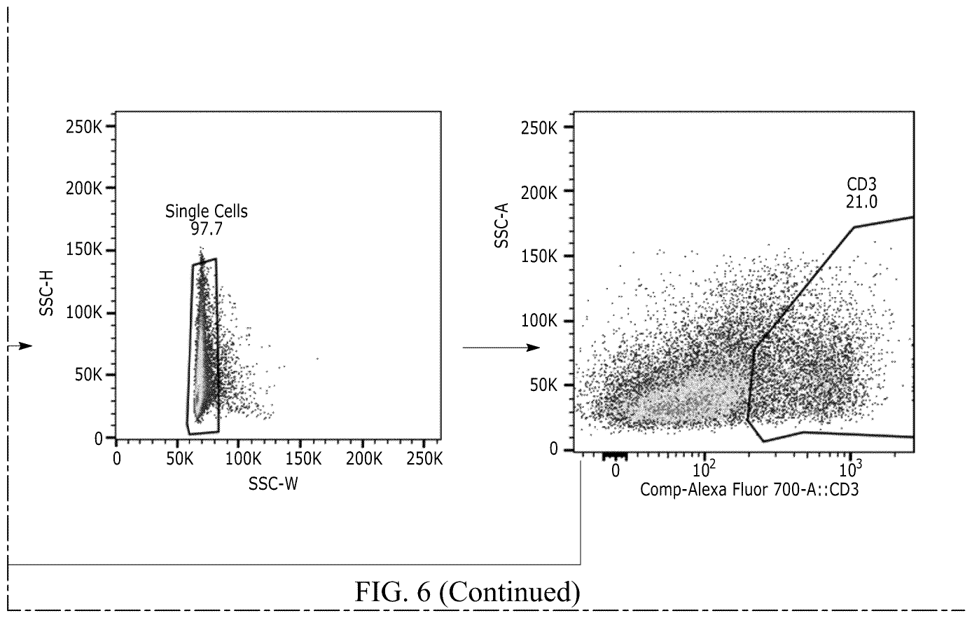

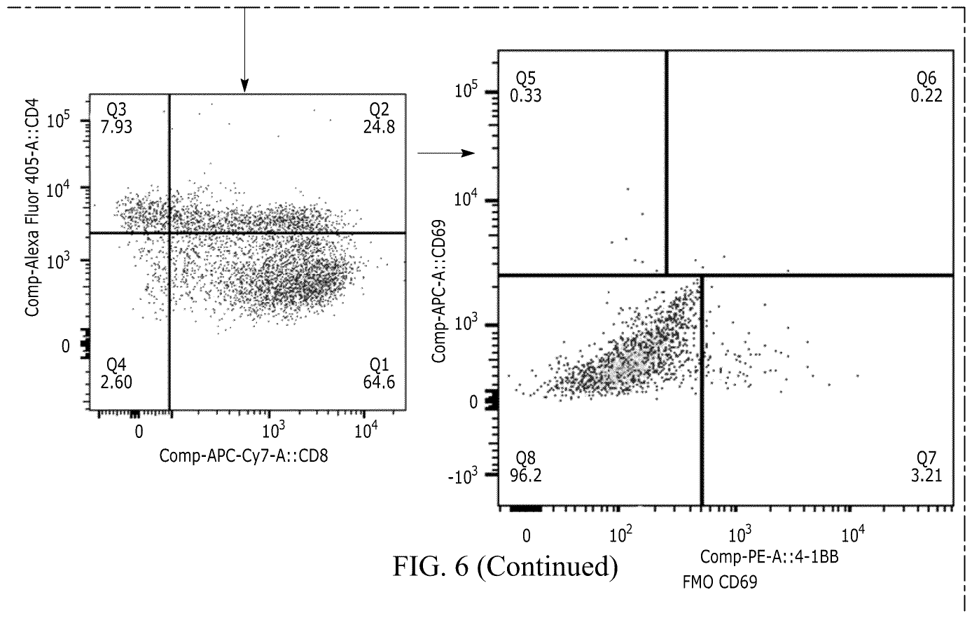

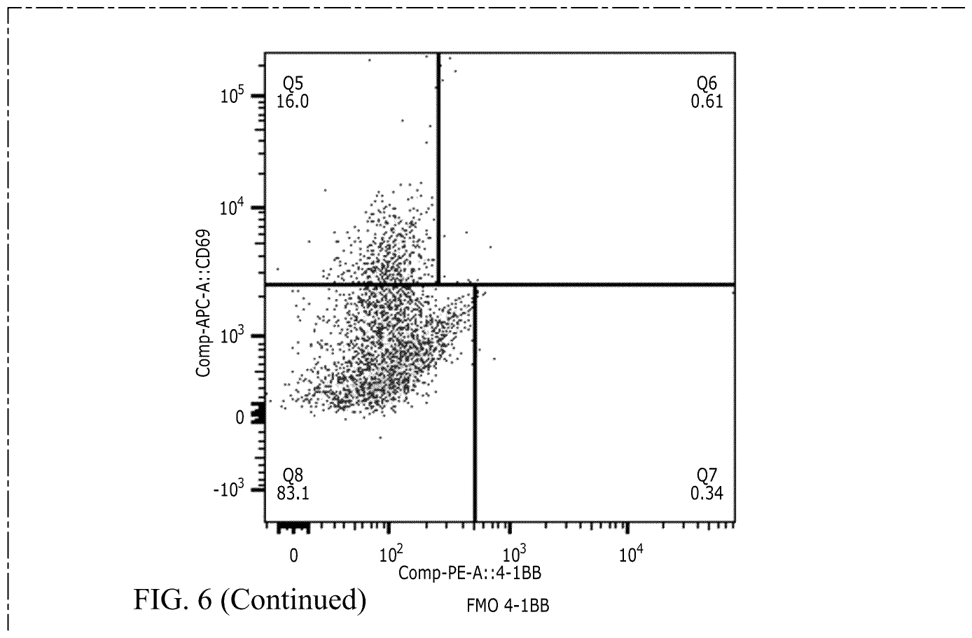

COVID mRNA vaccines are associated with generation of tumor-reactive T cells: Increases in myeloid activating cells and highly activated central memory and effector memory T cells suggested the expansion of tumor specific immune responses. However, to confirm that spike RNA-LNPs were mediating expansion of tumor reactive T cells, we leveraged an activation inducible marker (AIM) assay.¹⁸ In this assay, antigen reactive T cells cultured with tumor antigens ex vivo are identified through tandem expression of co-stimulatory molecules CD69 and 4-1BB (FIG. 3E, FIG. 6). Here, we co-cultured splenocytes from treated animals with overlapping peptide pools for melanoma associated peptide antigens gp-100, claudin 6, survivin, WT1, tyrosinase, Trp1, Trp2 and p53 and detected an increase in AIM+ CD8 T cells from animals treated with RNA-LNPs and ICI (FIG. 3F). These data confirm that spike mRNA LNPs prime the immune response for activation, presentation and recognition of tumor associated antigens in a manner that can be significantly expanded in through concomitant treatment with ICI.

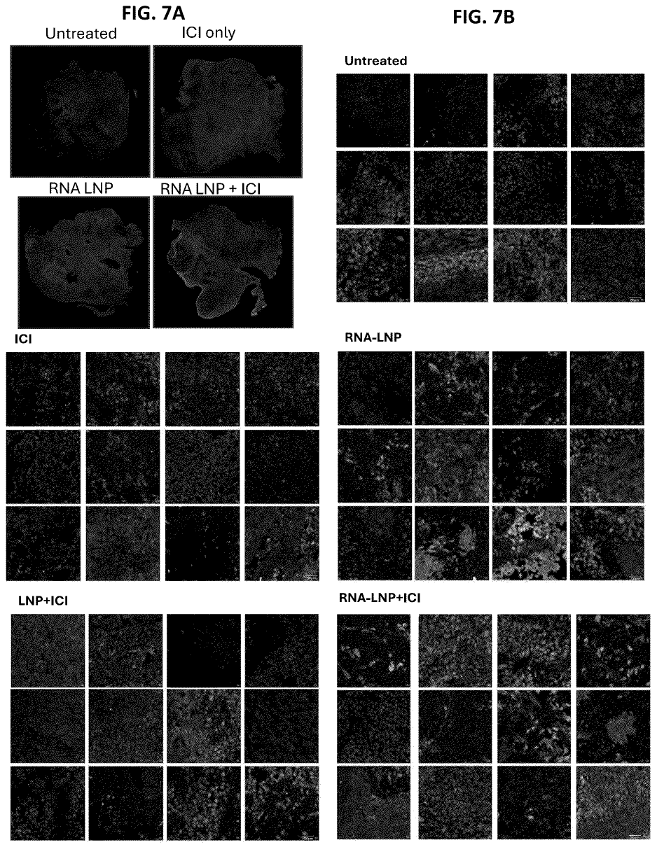

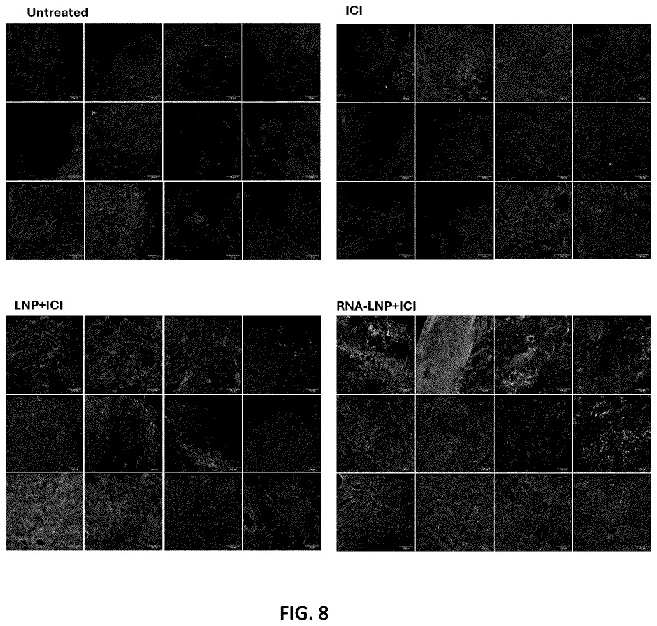

COVID mRNA vaccines increase intratumoral PD-L1 and expand PD-1+ TILs: To confirm whether these tumor-reactive cells entered tumors, we evaluated their direct impact on tumor cells. Upon recognition of tumors, tumor-reactive CD8 T cells secrete IFN-γ, which drives upregulation of PD-L1 expression on tumor cells. We found that spike encoding RNA-LNPs, especially when combined with PD-L1 blockade, significantly increased PD-L1 expression on tumor cells, supporting our hypothesis that antigen-specific CD8 T cells engaged tumor cells more frequently after mRNA immunization (FIG. 3G-3I, FIGS. 7A-7B). Moreover, the presence of ICI increases the expansion of tumor infiltrating lymphocytes (TILs) denoted by expression of CD3 and PD-L1 (FIGS. 3J-3I, FIG. 8).

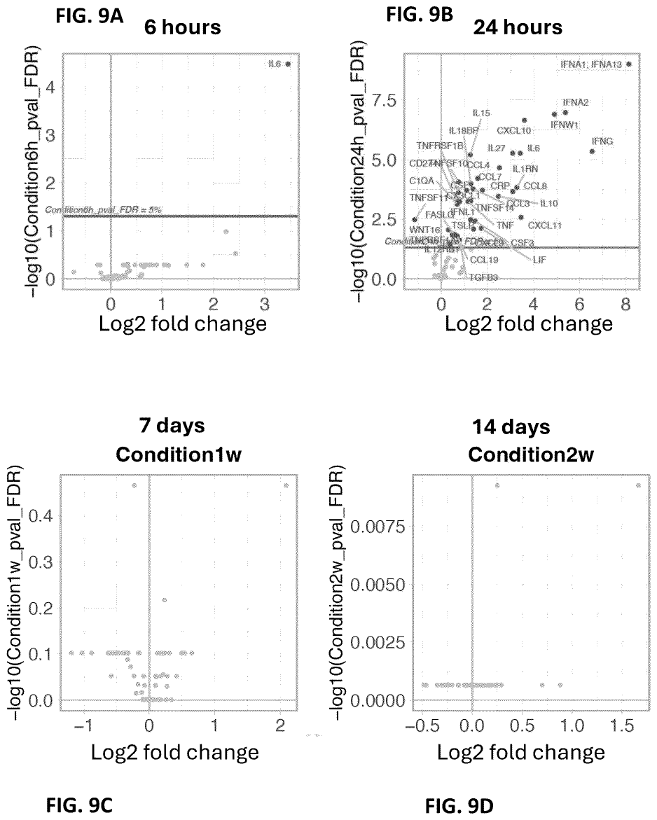

COVID mRNA vaccines produce broad cytokine responses in humans: Since there are species-specific differences in how humans and mice respond to mRNA, we sought to confirm that the pathway identified in our murine models were relevant in human systems. To do this, we collected blood and serum samples from five healthy volunteers at baseline and 6 hours, 24 hours, 7 days, and 14 days after receipt of a Moderna mRNA vaccine (FIG. 4A). We first evaluated serum from these volunteers with the NULISA-Seq Inflammation Panel (Alamar Biosciences), which is a multiplex assay designed to sensitively detect over 250 immune related cytokines. Interestingly, the only cytokine that was significantly elevated six hours after immunization was IL-6 (FIGS. 9A-9D). In contrast, a multitude of inflammatory cytokines surged at 24 hours after immunization, including IFN-α, IFN-γ, IFN-w, IFN-γ-inducible protein-10 (CXCL10), CXCL11, and IL-27 (FIG. 4B). Since IFN-α was found to be essential for antitumor activity in mice, we were particularly interested in the kinetics of this protein. Interestingly, not only was IFN-α upregulated, but it was the most upregulated cytokine at any timepoint, increasing by at least 6-fold relative to baseline in all five subjects (FIG. 4B-4D). Interestingly, although IL-1 was not found to be significantly increased at any timepoint, IL-1R antagonist (IL1RN) was significantly elevated at 24 hours, suggesting a compensatory response to a surge in IL-1 and IL-1R. Cytokine responses associated with COVID mRNA vaccination were short-lived in healthy subjects. All 26 cytokines that were significantly upregulated, 24 hours after immunization, returned to baseline by 7 days (FIG. 4C).

COVID mRNA vaccines activate innate immune cells in humans: To evaluate the impact of COVID mRNA induced antiviral cytokines on immune cells, we then evaluated the phenotypes of circulating myeloid cells by flow cytometry. Consistent with our findings in murine models, we found that mRNA immunization drove innate immune activation exemplified by increased expression of PD-L1 on circulating CD11b+ myeloid cells and increased expression of PD-L1, CD80, and CD86 on CD11c+ dendritic cells (FIG. 4E-4G). Vaccination was also associated with activation of natural killer cells exemplified by a doubling of expression of IL-2-Rα (CD25) on CD56hi cells (FIG. 4H) and circulating T cells exhibited a 30% increase in CD69 (FIG. 4I). This T cell activation did not persist at the 7 or 14 days timepoint, which may be a result of the lack of target in these healthy subjects or secondary to lymphoreticular localization for polarization into memory cells.

COVID mRNA vaccines are associated with increased tumor PD-L1 expression in patients with NSCLC: Since infiltration of antigen specific T cells in tumors is known to be associated with increases in PD-L1 expression on tumor cells and was correlated with antitumor response in our preclinical models, we hypothesized that patients who received a COVID mRNA vaccine would exhibit higher PD-L1 expression on their tumors. To test this hypothesis, we assembled two cohorts of patients. We first evaluated 2,358 pathology reports from patients with NSCLC with biopsies reporting tumor proportion score (TPS) of PD-L1 at MDACC between August 2019 and November 2023, separating patients into three groups based on the timing between their biopsy and their most recent mRNA vaccine. Patients who had received a COVID mRNA vaccine within 100 days of biopsy exhibited a 25% increase in mean tumor proportion score (TPS) of PD-L1 compared to patients who had not received any COVID mRNA vaccines prior to biopsy (30% vs 24%, p=0.041) and a 36% increase in mean TPS relative to patients who received an mRNA vaccine more than 100 days prior to biopsy (30% vs 22%, p=0.02) (FIG. 5B). Since TPS of 50% is a clinically important threshold to determine whether patients with NSCLC are eligible for single agent immunotherapy instead of chemoimmunotherapy, we then evaluated these data as a binary outcome around this threshold. Strikingly, we found that patients who received a COVID mRNA vaccine were 18% more likely to exceed the 50% TPS threshold over unvaccinated patients (33% vs 28%, p=0.039) (FIG. 5C), suggesting that mRNA vaccines have sufficient impact on TPS to modify treatment decisions. In contrast, pre-biopsy influenza and pneumonia vaccines were not associated with TPS changes (FIG. 5D).

COVID mRNA vaccines are associated with increased tumor PD-L1 expression in a broad patient cohort: Clinically, PD-L1 is measured in tumors in two ways. Tumor proportion score (TPS) quantifies PD-L1 expression on tumor cells, disregarding expression of PD-L1 on tumor-resident immune and epithelial cells. In contrast, combined proportion score (CPS) is quantified as the sum of the total numbers of PD-L1 expressing tumor cells and PD-L1 expressing immune cells within the tumor divided by the total number of tumor cells, with an arbitrary upper limit of 100%. Although TPS is associated with response to ICI in NSCLC, CPS is a more sensitive indicator of response to ICI in most other cancers. To expand our findings on TPS beyond NSCLC and evaluate the impact of mRNA vaccines on CPS, we assembled a separate cohort of patients including all patients at a quaternary referral center with pathology reports including the term “PD-L1” in a four-year period including the pandemic era (FIG. 5E). Altogether, we identified 5,524 pathology reports with the term “PD-L1” including 2,231 reporting TPS and 3,309 reporting CPS from a diverse array of patients representing a variety of primary sites and histologies (FIG. 5f). In this cohort, receipt of a COVID mRNA vaccine within 100 days prior to biopsy was associated with a 70% increase in TPS (17% vs 10%, p<0.001) (FIG. 5G), twice the effect seen in NSCLC patients. In addition, CPS was elevated by 26% in patients who had received a vaccine within 100 days prior to biopsy relative to those who did not receive a vaccine (17% vs 13%, p=0.006) (FIG. 5g). As in the NSCLC cohort, influenza vaccines were not associated with similar increases in TPS and CPS (FIG. 5H).

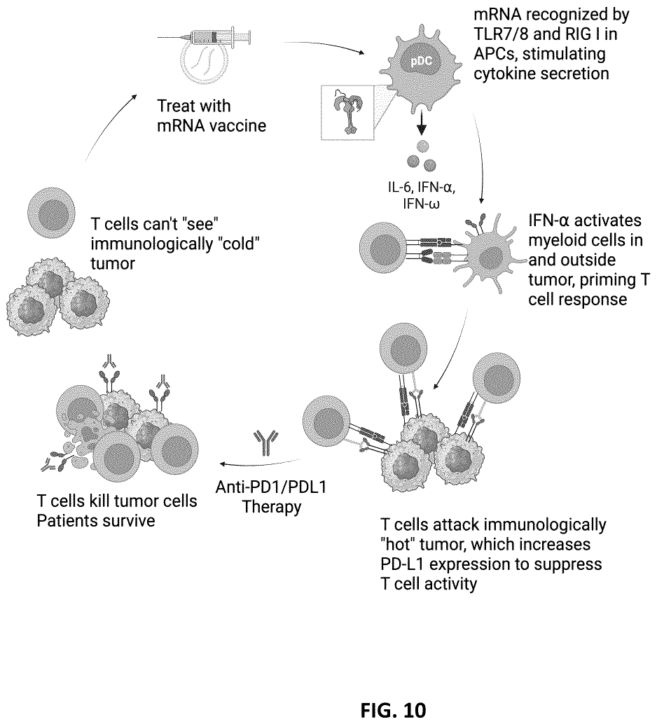

Together, this data suggests a model in which COVID mRNA vaccines generate a surge in antiviral cytokines including IFN-α that drive systemic innate immune activation (FIG. 10). Tumor resident innate immune cells present antigen to T cells, which then secrete IFN-γ in tumors leading to increased PD-L1 expression by those tumors and increased PD-1+ effector T cells following ICI treatment. In combination with ICI, COVID mRNA vaccines prime systemic immune response and overcome compensatory intratumoral PD-L1 expression eliciting tumor regression and improved survival.

### Example 2: Improved Survival Outcomes in NSCLC Patients

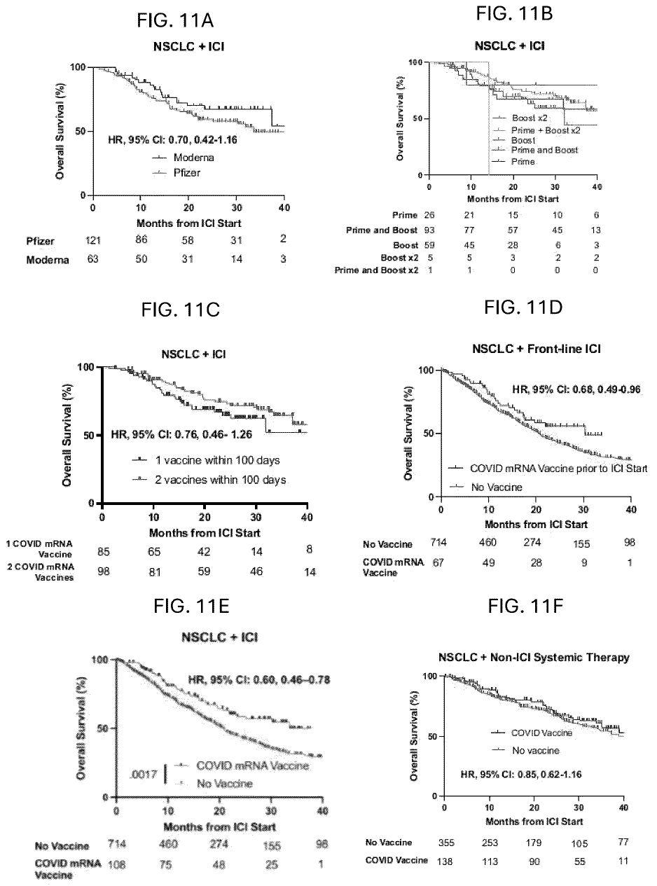

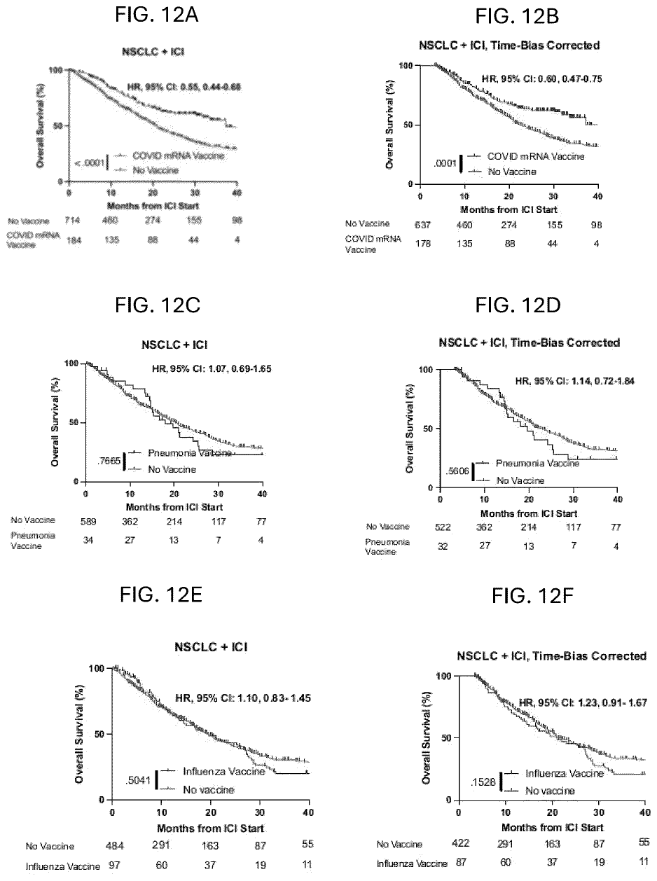

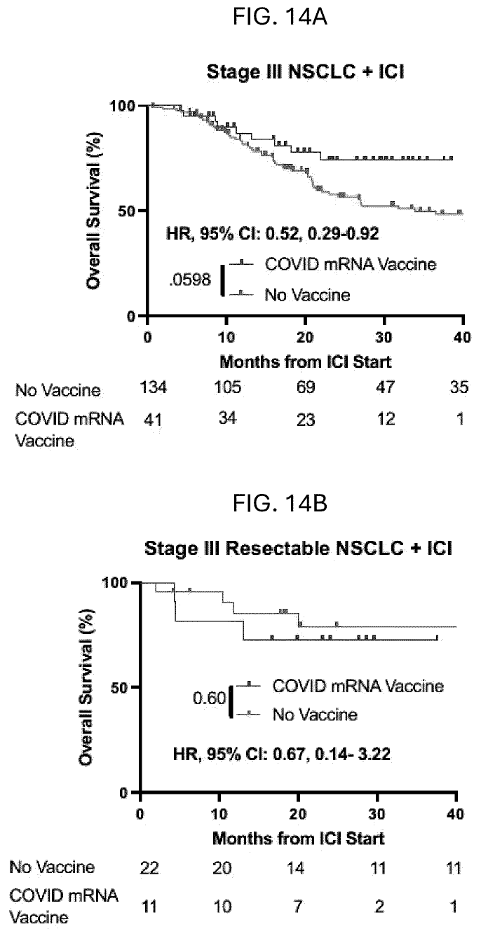

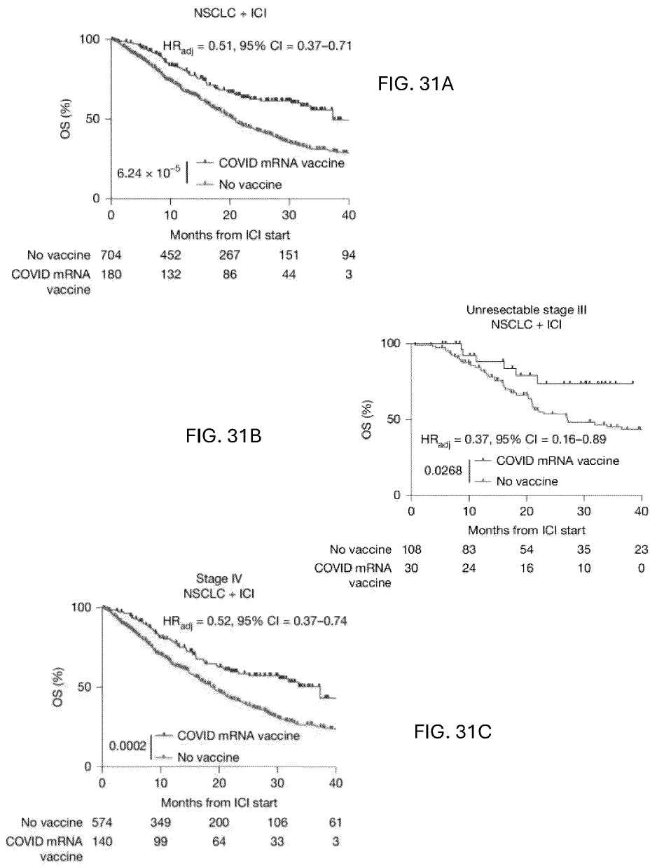

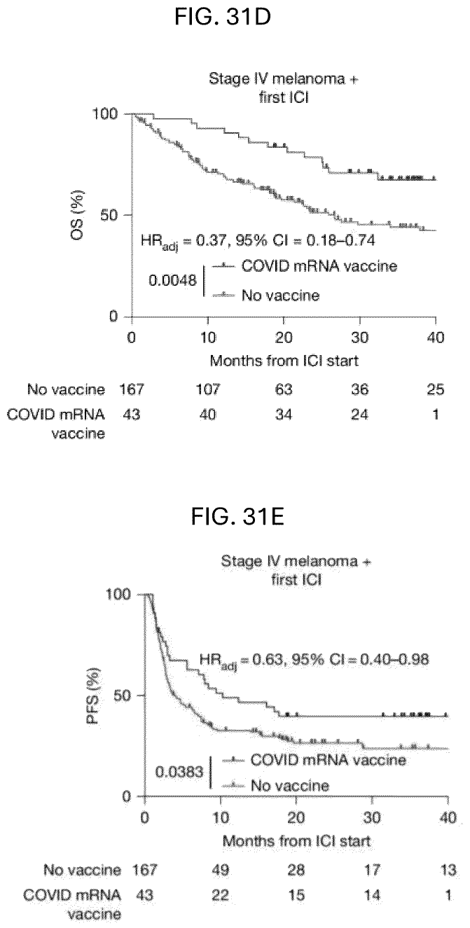

To determine whether COVID mRNA vaccines were associated with improved responses to immune checkpoint blockade, the overall survival was compared among a cohort of patients with Stage III/IV non-small cell lung cancer (NSCLC) treated at MD Anderson Cancer Center (MDACC) between August 2019 and August 2023 (Table 1). 180 patients were identified who received an mRNA vaccine within 100 days of ICI initiation, and 704 patients were identified who were treated with ICI and did not receive an mRNA vaccine (Table 1). Of these, 117 patients received the BNT162b2 vaccine and 63 received mRNA-1273 (FIG. 11A). 24 patients received a priming dose only, 57 patients received a “booster” only, 93 patients received both a prime and a boost dose. Five patients received two “booster” doses, and one patient received a priming dose and two booster doses (FIG. 11B). 81 patients received 1 dose of COVID mRNA vaccination within 100 days, 98 patients received 2 doses, and 1 received 3 doses (FIG. 11C).). After controlling for 39 covariables with Cox proportional hazards regression, including clinical stage, histology, steroid use, performance status, mutation status, comorbidities and treatment year, in this dataset, it was discovered that receipt of a COVID mRNA vaccine within 100 days of initiation of treatment with ICI was associated with significantly improved median OS (20.6 months versus 37.3 months) and 3 year OS (30.8% versus 55.7%, adjusted hazard ratio (HRadj=0.51, 95% confidence interval (Cl)=0.37-0.71, P<0.0001)) (FIG. 31A). This survival advantage was similar for patients with Stage III Unresectable NSCLC (HR_{adj}=0.37, 95% Cl=0.16-0.89, P=0.0268) (FIG. 31B) and Stage IV NSCLC (HR_{adj}=0.52, 95% Cl=0.37-0.74, P=0.0002) (FIG. 31C). This was true for patients who received mRNA vaccines from either vaccine manufacturer (FIG. 11A) and for patients who had or had not received a previous COVID mRNA vaccine (FIG. 11B). Patients who received two vaccines in the 100 days surrounding initiation of ICI experienced numerically improved but statistically insignificant improvements in overall survival compared to those who received only one vaccine (FIG. 11C). These results were also consistent when considering only those patients who received first-line ICI and received their COVID-19 vaccine prior to initiating ICI (HR 0.68, 95% Cl 0.49-0.96) (FIG. 11D), after correcting for immortal time bias (FIGS. 12A-12B), when narrowing the vaccination window to 50 instead of 100 days (FIG. 11E), and with propensity score matching (FIGS. 3A-3B). Patients who received a COVID-19 vaccine within 100 days of chemotherapy (a group that did not include targeted therapies owing to significant heterogeneity and limited patient numbers within different drug cohorts) but did not receive ICI had no detectable survival benefit. (Data not shown.) In contrast, patients who received pneumonia or influenza vaccines within 100 days of initiating ICI (FIGS. 12C-12F), those who received COVID vaccines but did not receive ICI, and those with resectable Stage III tumors experienced no detectable survival benefit (FIG. 11F, FIGS. 14A-14B).

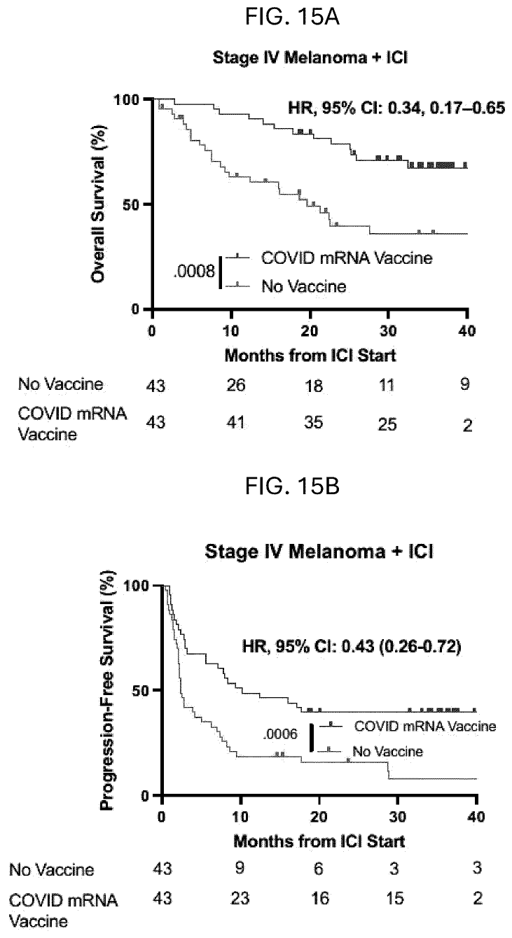

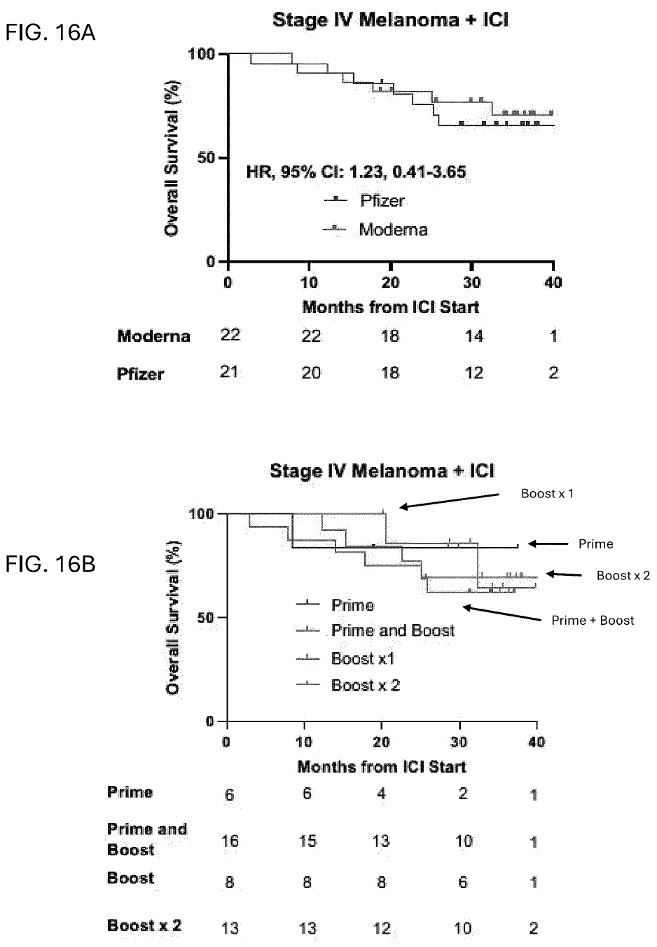

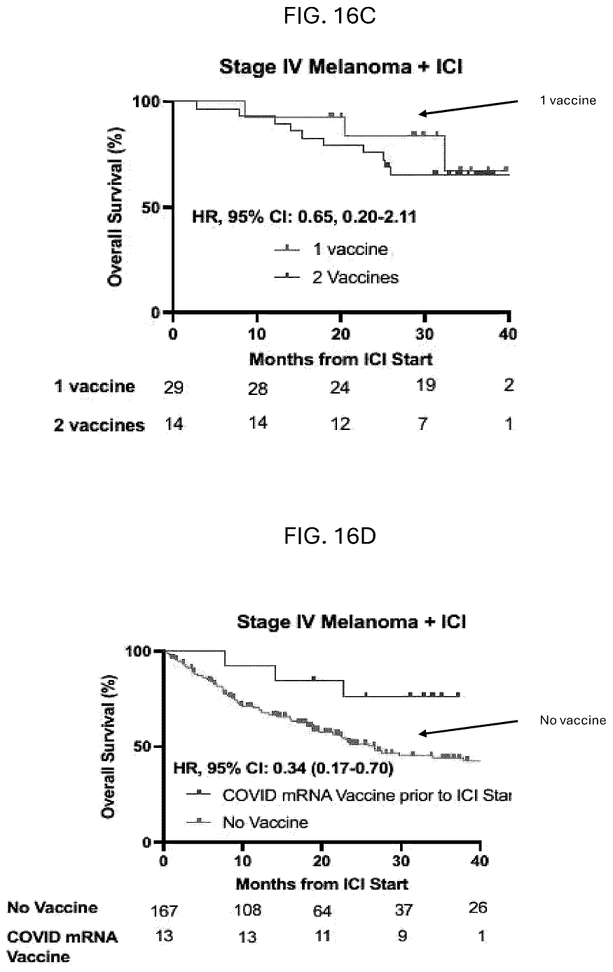

This analysis was repeated in a separate cohort of patients treated with front-line ICI for metastatic melanoma, including 43 patients who received an mRNA vaccine within 100 days of initiating ICI and 167 who did not receive an mRNA vaccine (Table 4). Of the patients who received mRNA vaccines, 21 patients received BNT162b2 and 22 patients received mRNA-1273 (FIG. 16A). Six patients received a priming dose only, 8 patients received a “booster” only, 16 patients received both a prime and a boost dose, and 13 patients received two “booster” doses, (FIG. 16B). 29 patients received 2 doses of COVID mRNA vaccination within 100 days, and 14 patients received 1 dose (FIG. 16C). After accounting for 28 covariates with Cox Proportional Hazards Regression (Tables 13-16), it was found that receipt of a COVID mRNA vaccine within 100 days of initiating ICI was associated with substantially improved OS (median OS, 26.67 months versus unmet; 36-month OS, 44.1% versus 67.6%; HR_{adj}=0.37, 95% Cl=0.18-0.74, P=0.0048) and numerically improved progression free survival (PFS) (median PFS, 4.0 months versus 10.3 months; 36-month PFS, 23.7% versus 39.5%; HR_{adj}=0.63, 95% Cl=0.40-0.98, P=0.0383). These effects were magnified with propensity score matching (OS: HR=0.44, 95% Cl=0.18-0.77, P=0.0063; PFS: HR=0.46, 95% Cl=0.27-0.77, P=0.0022) (FIGS. 15A-15B) and were again similar for both vaccine manufacturers, prime and boost vaccines, patients receiving single or multiple vaccine doses, and when limiting the analysis to only patients who received their vaccine prior to initiating ICI or only those treated during the pandemic, and when expanding the analysis to include patients on second or third line therapy and those without distant metastases (FIGS. 16A-18C).

The combination of SARS-CoV-2 mRNA vaccines and ICIs resulted in markedly improved overall survival (OS) in patients with Stage III/IV non-small cell lung cancer (NSCLC).

Patients who received the vaccine achieved a near doubling of median OS, improving from 20.7 months to 37.33 months. Three-year survival rates increased from 30.8% (unvaccinated) to 55.7% (vaccinated, (HR_{adj}=0.51, 95% confidence interval (Cl)=0.37-0.71, P<0.0001)) (FIG. 1A or FIG. 31A). Subgroup analyses revealed survival advantages across Stage III unresectable NSCLC (HR_{adj}=0.37, 95% Cl=0.16-0.89, P=0.0268) (FIG. 1B or FIG. 31B) and Stage IV NSCLC cohorts (HR_{adj}=0.52, 95% Cl=0.37-0.74, P=0.0002) (FIG. 1C or FIG. 31C). The benefits were independent of manufacturer (BNT162b2 or mRNA-1273) and vaccine timing relative to ICI initiation.

Additional analyses were performed using Cox proportional hazards regression, which accounted for covariates like tumor stage, vaccine timing, and ECOG performance score. Propensity score matching further validated the survival advantages by balancing confounding variables like steroid use, radiation therapy, and tumor mutation profiles between vaccinated and unvaccinated groups.

Survival benefits were specific to COVID mRNA vaccines, as patients who received influenza or pneumonia vaccines within comparable time frames did not exhibit improved survival outcomes (FIG. 12C-12F). This specificity underscores the immunomodulatory effect of nanoparticle-based mRNA vaccines (e.g., SARS-CoV-2 mRNA vaccines), offering a novel adjunct to ICI therapy for NSCLC patients.

### Example 3: Enhanced Survival in Metastatic Melanoma

The analysis was repeated in metastatic melanoma patients treated at MDACC between January 2019 and August 2023. Metastatic melanoma patients also showed substantial improvements in OS and progression-free survival (PFS) when COVID mRNA vaccines were administered within 100 days of ICI therapy. The retrospective cohort included 43 vaccinated patients, and 167 unvaccinated control patients treated with ICIs between January 2019 and August 2023 at MDACC.

Median OS for vaccinated patients reached 26.67 months compared to an unmet survival endpoint in the unvaccinated cohort, with three-year survival rates increasing from 43.7% (unvaccinated) to 67.2% (HR=0.329, 95% Cl 0.16-0.67, p=0.002) (FIG. 1D-1E or FIGS. 31D-31E). Although initial PFS analysis showed limited statistical significance (median PFS: 4 months vs. 10 months), comprehensive matching using propensity scores revealed significant improvements under vaccination (HR=0.42, p<0.001; FIGS. 15A-15B).

Data collection included patient demographics, mutations (e.g., BRAF, RAS), metastatic burden, treatment cycles (single-agent or combination ICIs), and vaccine administration specifics. These secondary analyses revealed survival enhancements independent of vaccine manufacturer (FIGS. 16A-16C) and irrespective of booster dose timing.

Parallel results in the NSCLC and melanoma cohorts underscore the utility of SARS-CoV-2 mRNA vaccines in increasing ICI efficacy through systemic immune modulation and tumor microenvironment priming.

### Example 4: Synergistic Effects in Murine Models

To demonstrate whether effects observed in humans could be modeled in animals, formulations were recreated in commercial preparations for administration to tumor bearing animals in conjunction with ICI. Since SARS-CoV-2 spike protein has been fully sequenced, the published mRNA construct utilized for the BioNTech vaccine (BNT162b2) was synthesized and the fidelity of synthesis was validated based on mRNA size and ability to elicit neutralizing antibodies following in vivo administration to animals (data not shown). Administrations were formulated in lipid-nanoparticles (LNPs) with 100% efficiency and met specifications delineated for clinical preparations of BNT162b2 based on size distribution, polydispersity and charge (data not shown)^{20,21}, the latter of which could be found to be disproportionately affected by buffer conditions leading to net positive charge.

Murine models provided preclinical validation for the immunological mechanisms underlying enhanced ICI sensitivity due to COVID mRNA vaccination. Experiments were conducted under protocols approved by the University of Florida and MD Anderson Institutional Animal Care and Use Committees (IACUC). C57Bl/6 mice were inoculated with B16F0 melanoma or Lewis Lung Carcinoma (LLC) cells and vaccinated using intramuscular doses of Spike RNA lipid nanoparticles (RNA-LNPs).

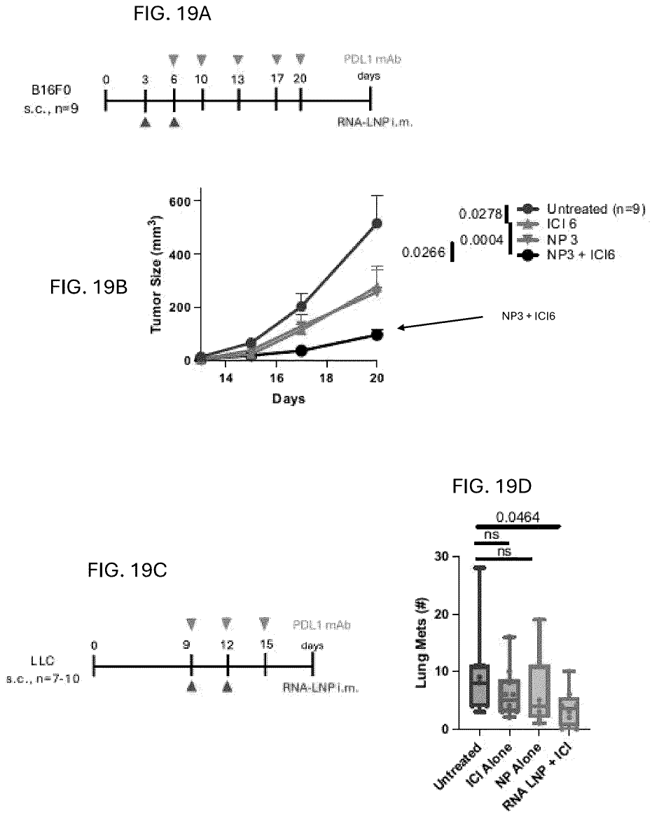

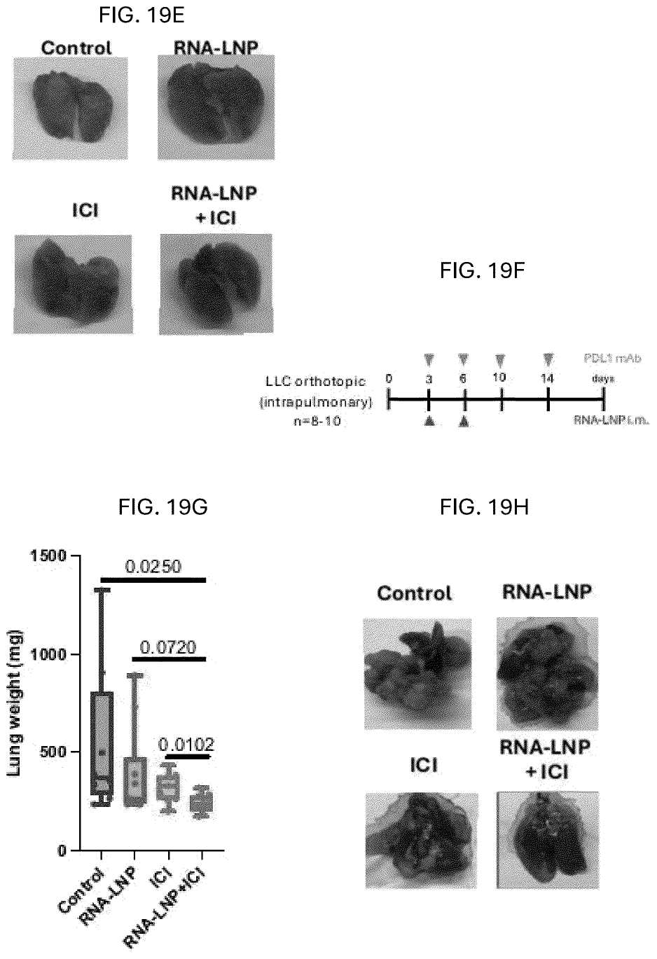

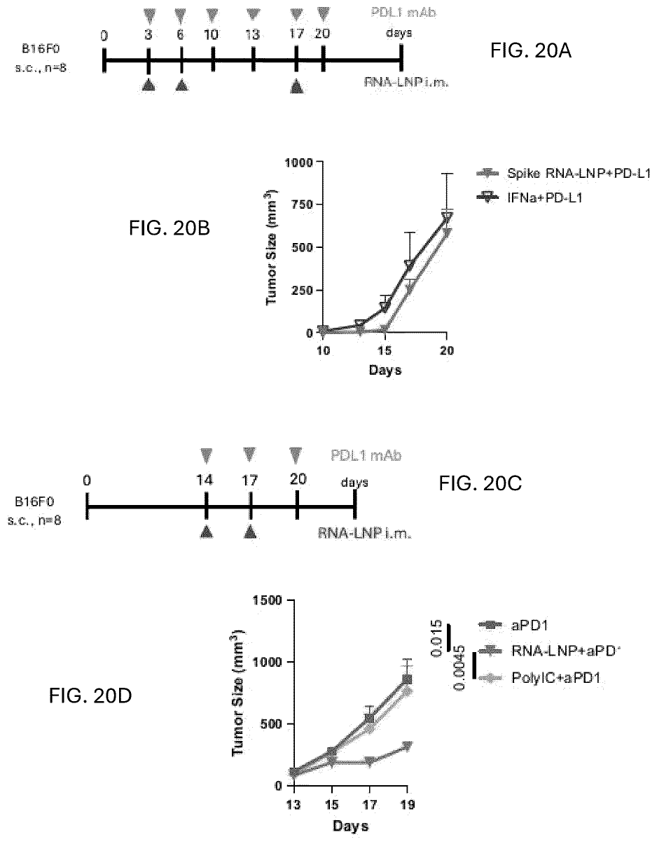

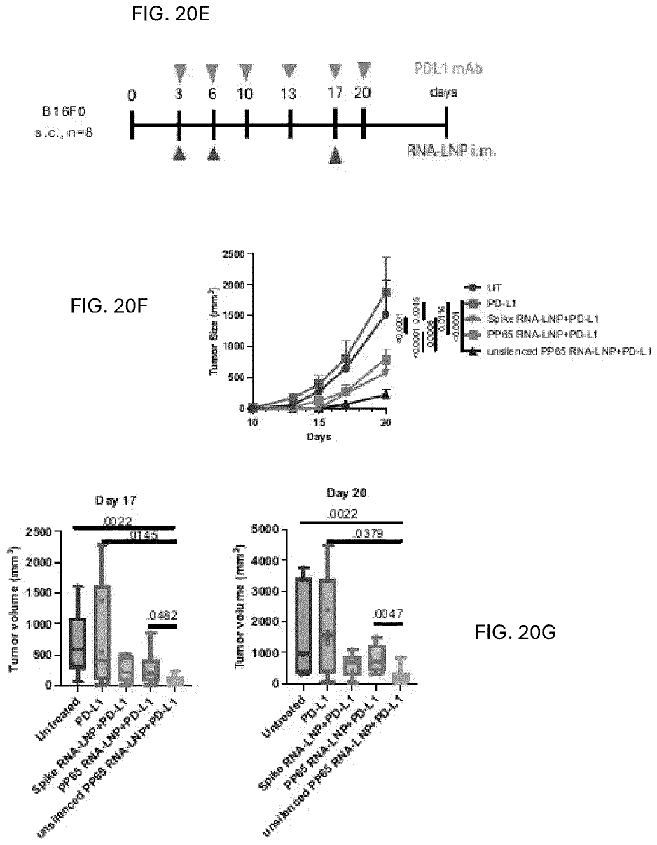

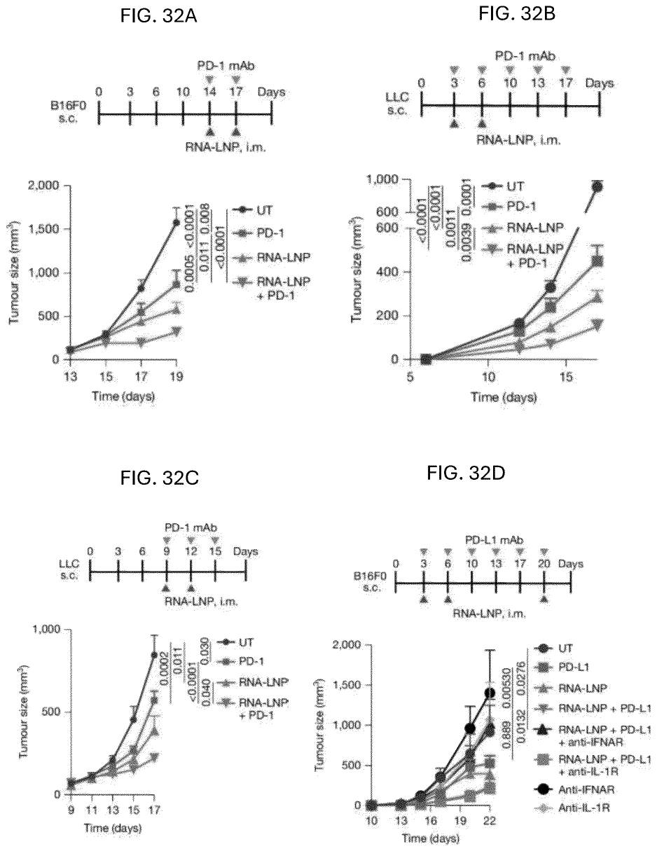

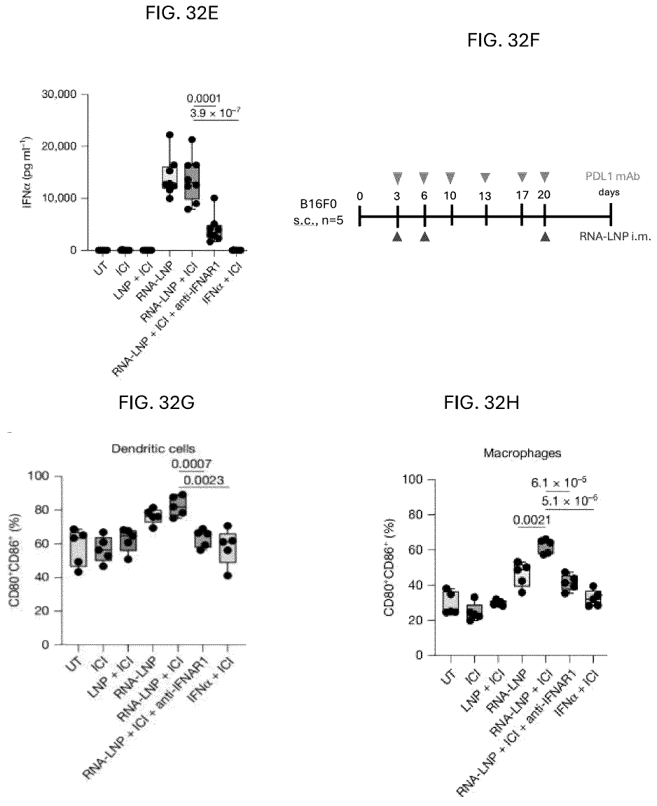

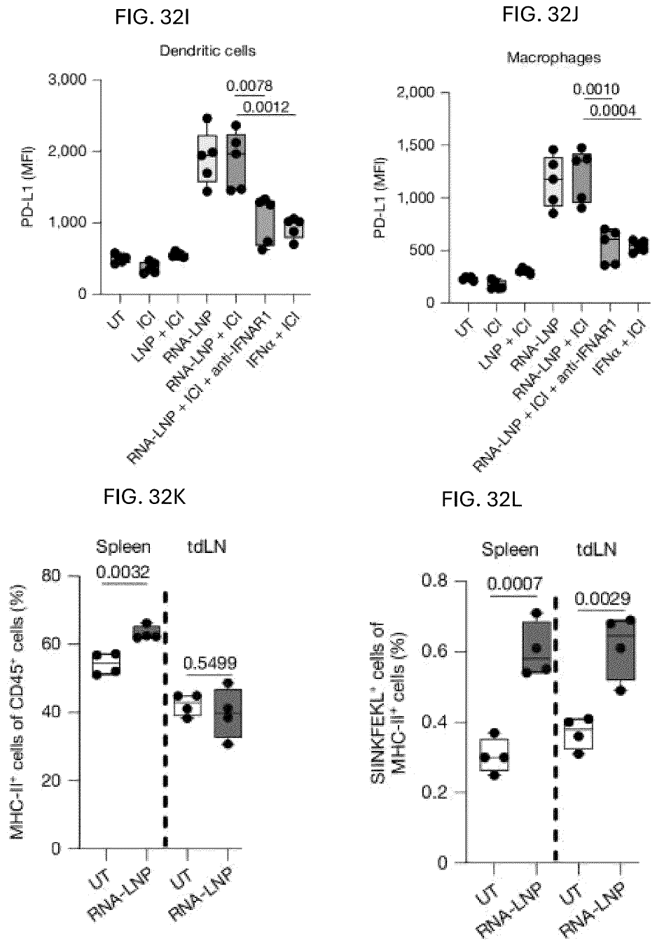

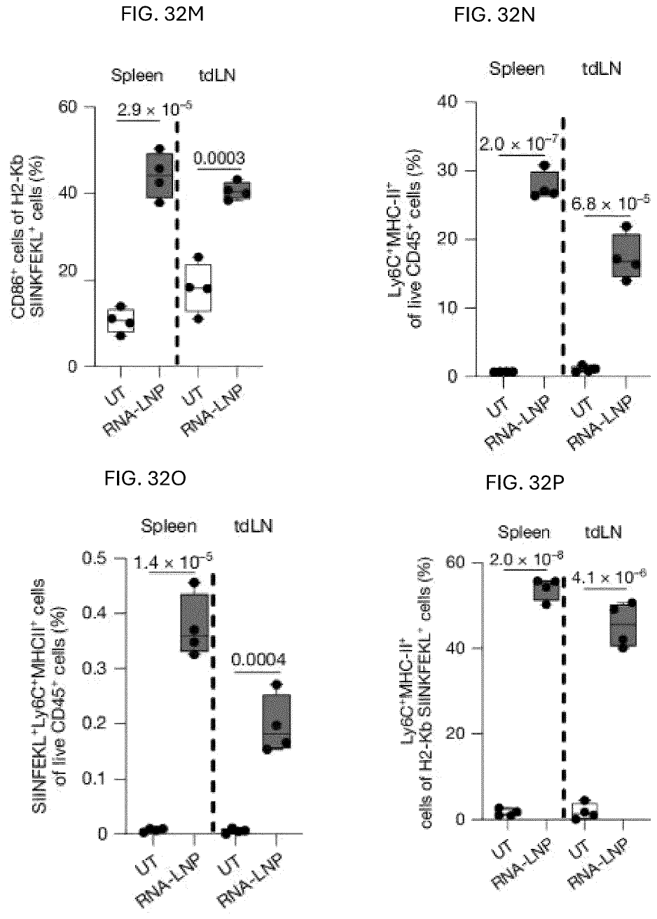

Murine B16F0 melanoma and Lewis Lung Carcinoma were chosen as models to test immunogenicity and efficacy of spike RNA-LNP vaccines due to effects of the vaccine in the clinical setting and because both tumor models are poorly responsive to ICIs. First, mice were treated with two vaccine doses in conjunction with ICI treatment. This regimen was found to be superior to either monotherapy alone in mice with established B16F0 tumors with tumor volumes ˜80 mm³ (FIGS. 32A-B), in mice with s.c. LLC (FIGS. 32C-32D), and in mice with established s.c. LLC with tumor volumes ˜100 mm³ (FIGS. 32E-32G). It was also found that starting RNA-LNPs prior to ICI produced similar effects relative to concomitant treatment (FIGS. 19A-19B). In mice with established LLC, metastatic lesions were identified in the lungs of untreated mice that were similar for monotherapy, but significantly reduced with the combination treatment (FIGS. 19C-19E). Based on this observation, it was then tested whether RNA-LNPs could reduce the growth of intrapulmonary tumors. LLC cells were implanted orthotopically and treatment was administered starting on Day 3. In this model, the combination of RNA-LNPs and ICI resulted in superior inhibition of tumor growth as measured by lung weights (FIGS. 19F-19H). To understand the mechanism by which RNA-LNPs targeting the Spike protein mediate these antitumor effects, an early treatment model of B16F0 was utilized with or without cytokine-blocking antibodies. In this model, RNA-LNPs and PD-L1 blockade each provided numerical but statistically insignificant survival benefit. However, combination therapy with RNA-LNPs and PD-L1 blockade strongly inhibited tumor growth (FIGS. 32H-321). Although a role for IFN-α in the response to mRNA vaccines was previously demonstrated, recent evidence suggests a dominant role for IL-1 signaling in responses to the specific RNA-LNPs targeting the COVID spike protein.¹⁴ The importance of each pathway was evaluated with antibodies blocking the IL-1 and IFN-α receptors. While blockade of IL-1R had no effect on tumor growth, anti-tumor responses were completely abrogated with pre-administration of type I interferon signaling with IFNAR1 monoclonal antibodies (FIGS. 32H-321). In addition, direct administration of supraphysiologic doses of type I interferon recapitulated antitumor effects (FIGS. 20A-20B). However, stimulation of type I interferon signaling with LMW poly IC failed to elicit similar immunity (FIGS. 20C-20D). These results highlight the importance of interferon in driving innate and adaptive immunity, and the ability to manipulate and reset the immune setpoint away from tolerance toward effector immune responses in the presence of ICIs using a commercially available mRNA preparation.

RNA-LNP vaccines combined with ICIs elicited robust tumor inhibition in both subcutaneous and orthotopic tumor models, surpassing the efficacy of monotherapy (RNA-LNP or ICI alone) (FIGS. 32A-32G). Tumors treated with both modalities exhibited decreased volumes (80-100 mm³ for LLC) and reduced metastatic burden compared to untreated controls (FIGS. 19A-19H).

Type I interferon signaling emerged as critical for eliciting anti-tumor effects. Blocking interferon-α receptors with monoclonal antibodies (IFNAR1) abolished the survival benefit induced by RNA-LNP vaccination (FIGS. 32H-32I). Flow cytometry quantified immune activation in lymphoid organs, revealing APC migration and activation (e.g., dendritic cells expressing MHC-II) upon interferon stimulation. Immunofluorescence imaging validated PD-L1 reductions in tumor cells post-therapy (FIGS. 32J-32O).

These findings corroborate the importance of the vaccine-induced cytokine surge in driving APC activation and sensitizing immune-resistant tumors to ICI therapy.

### Example 5: Testing Other mRNA Species for Antitumor Effects

It was assessed whether mRNA species other than those described above would elicit similar antitumor effects. The protein encoded by the mRNA was modified by replacing the spike mRNA with mRNA encoding the cytomegalovirus antigen pp65, which is overexpressed in glioma but not expressed in B16F0. No significant difference was found in antitumor activity between these two groups, suggesting that innate immune sensing of the mRNA itself, not the protein encoded by the mRNA, is the primary driver of antitumor activity from mRNA vaccines (FIGS. 20E-20G). Since N1-methyl-pseudouridine is used in COVID mRNA vaccines to “silence” innate immune activation, it was hypothesized that “unmodified” mRNA with fully intact motifs to activate pattern recognition receptors would provide even more robust innate immune activation and antitumor activity. Replacing “silenced” pp65 mRNA with “unsilenced” pp65 mRNA resulted in further synergy with ICI, again suggesting that innate sensing of mRNA is a critical driver of antitumor responses (FIGS. 20E-20G).

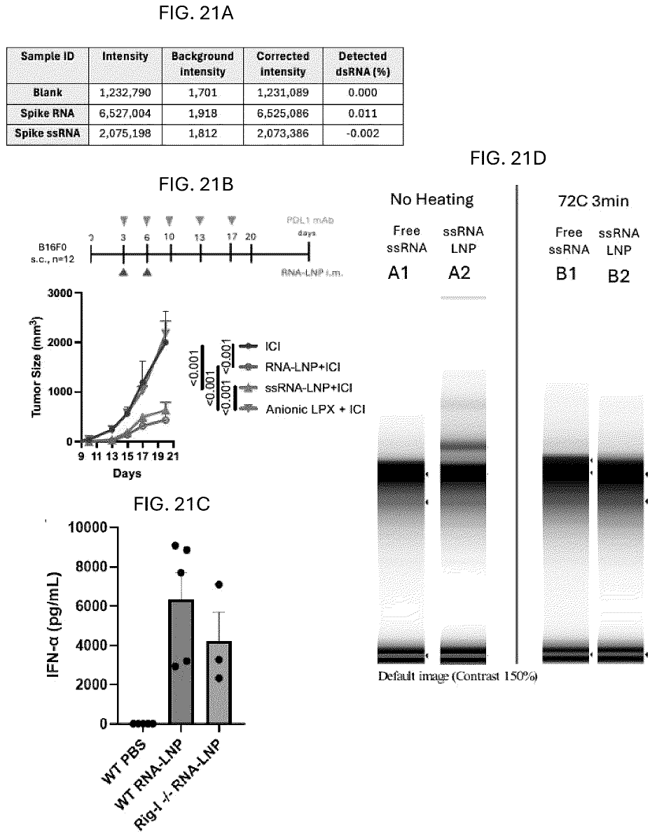

Additional studies were completed to identify how RNA-LNPs elicit antitumor immunity. RNA-LNPs stimulate type I interferon production by stimulating the intracellular dsRNA sensor MDA5.22. However, the mechanism by which RNA-LNPs activate this sensor is unknown. To understand this further, a role for dsRNA in our vaccines was ruled out by measuring the level of dsRNA contamination in our manufactured ssRNA product. The dsRNA/ssRNA ratio in the mRNA was found to be 0.011% (FIGS. 21A-21D). The tumor growth curve with an additional purification step was repeated. With this step, a reported dsRNA/ssRNA ratio of −0.002% was achieved (FIG. 21A). Importantly, no change in antitumor efficacy was found with the complete elimination of dsRNA using this method (FIG. 21B). Together, this data suggests that dsRNA contamination is not a major cause of the anti-tumor effects in our preclinical models. Next, it was tested whether the specific LNP construct impacted antitumor immunity with the same mRNA input. It was found that anionic lipoplexes (LPX) do not elicit similar antitumor effects. It was hypothesized that RNA-LNPs might create higher order structures as it has been described that higher-order RNA structures may also stimulate MDA5. In line with this hypothesis, it was found that RNA extracted from our RNA-LNPs contained high molecular weight secondary structures similar in mass to dsRNA (FIG. 21C). Combined with previous reports of MDA5 activation with RNA-LNPs but not with ssRNA alone, this data may suggest a mechanism by which encapsulation with LNPs forms secondary structures enabling activation of double stranded sensing machinery (i.e., MDA5 activation) for induction of type I interferons. COVID mRNA vaccines activate innate immune cells and stimulate them to present of tumor antigens.

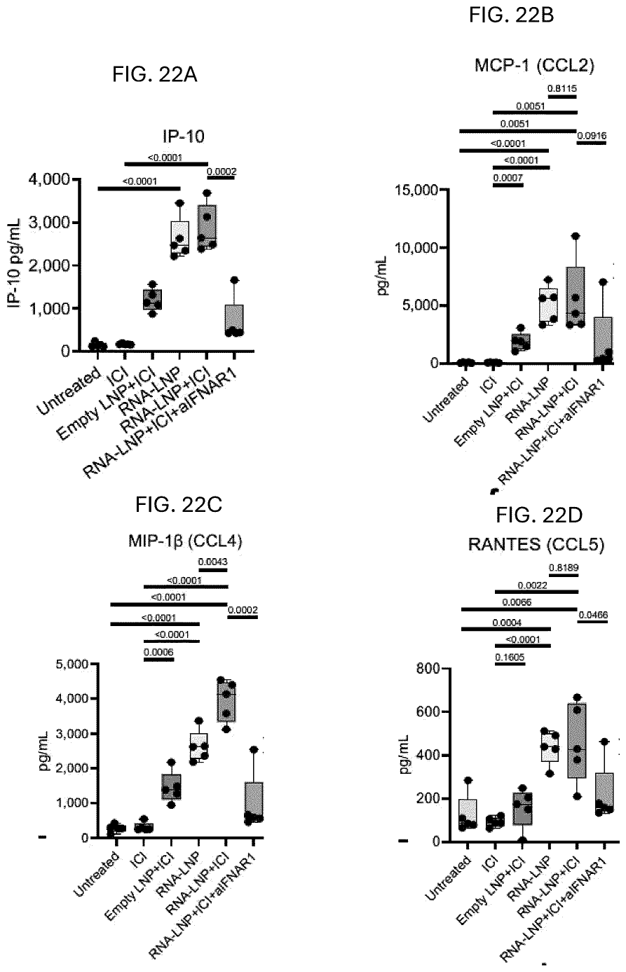

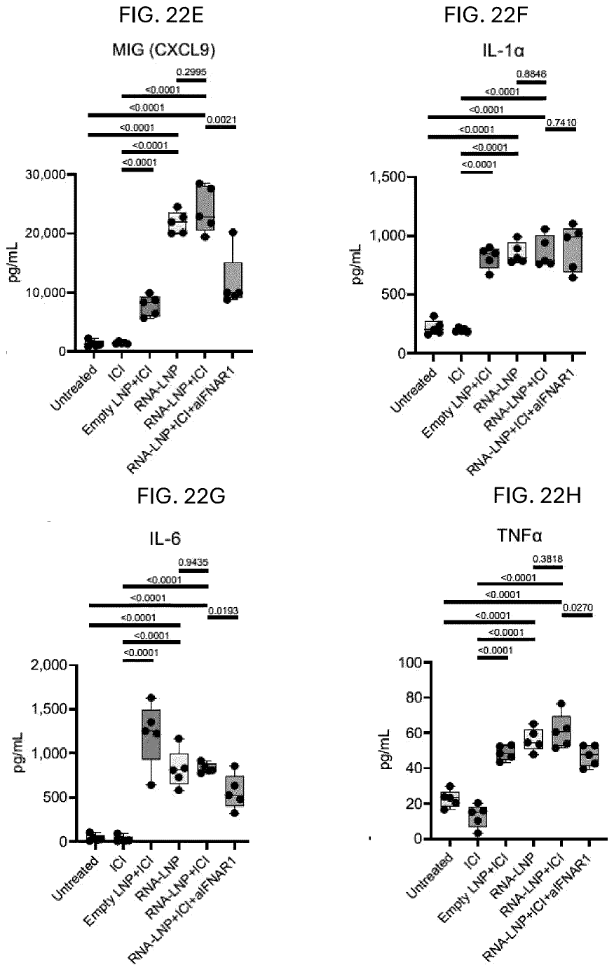

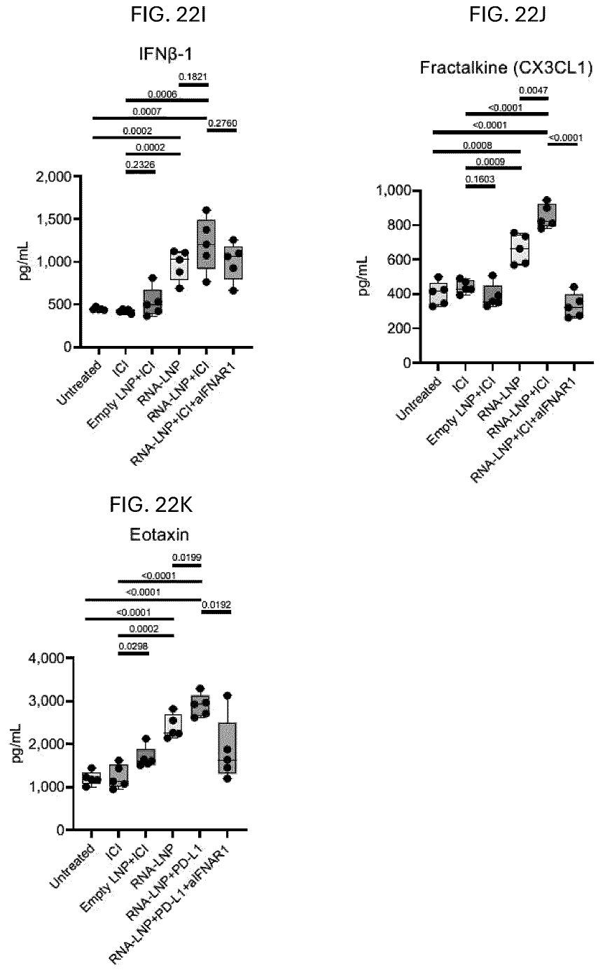

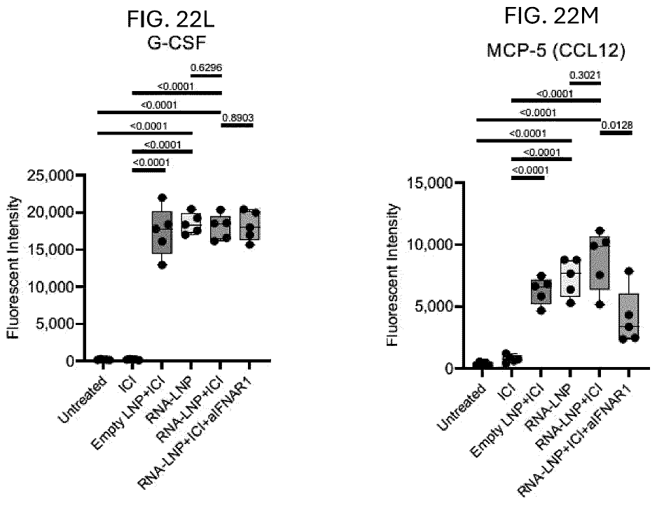

It was sought to better understand the impact of RNA-LNPs on innate immune cells. IFN-α was dramatically elevated in both RNA-LNP and combination groups in conjunction with other Th1 chemokines (FIG. 32K, or FIGS. 22A-22M). This surge in cytokine/chemokine response correlated with an increase in myeloid cell activation in the lymphoid organs of animals treated with RNA-LNP alone or in combination with ICI. Following spike encoding RNA-LNP, there was precipitous activation of antigen presenting cells (APCs) including dendritic cells, macrophages and inflammatory monocytes expressing MHC class-II in lymphoreticular organs that was dependent on IFN-α-receptor (IFNAR1) (FIGS. 32L-32O). While this response was dependent on interferon signaling, the magnitude of the response could not be achieved by simply administering systemic IFN-α in combination with ICI (FIGS. 32L-32O). In tandem, myeloid activation extended to the tumor, where receipt of RNA-LNPs was associated with IFNAR1-dependent increases in activated CD11b+ myeloid cells and Ly6C+ myeloid cells (data not shown). To understand the function of these cells, mice were treated with B16F10 tumors expressing chicken ovalbumin (B16F10-OVA) at Days 10 and 13 and APC localization and presentation of ovalbumin was evaluated with flow cytometry. It was found that RNA-LNPs stimulate APCs to migrate to lymphoid organs and present tumor antigens in the presence of costimulatory molecules (FIGS. 32I-32O). Interestingly, it was found that this migration and presentation of tumor antigens in the presence of costimulatory molecules was particularly enriched in Ly6C⁺MHC-II⁺ cells (FIGS. 32N-32P).

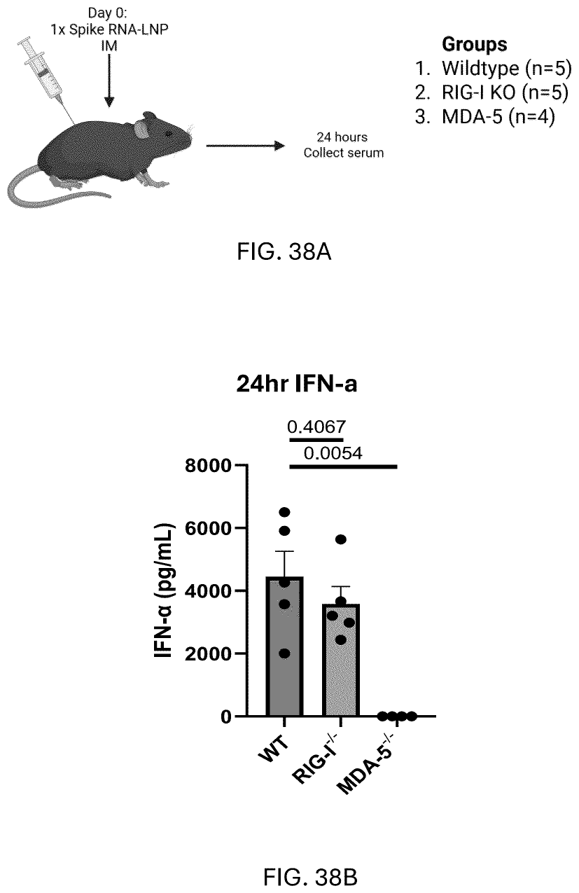

MDA5 (Melanoma Differentiation-Associated Protein 5) is an intracellular molecular sensor that detects high molecular weight (“HMW”) nucleic acid structures. RIG-I (Retinoic acid-inducible gene I) is an intracellular molecular sensor that detects low molecular weight (“LMW”) nucleic acid structures. Wild-type, RIG-I KO, or MDA-5 KO (MDA-5) mice were treated with RNA-LNPs. Serum was harvested 24 hours post treatment and an ELISA was performed for IFN-α (FIG. 38A). In mice that lack MDA-5, IFN-α levels were significantly decreased compared to wild-type and RIG-I KO mice (FIG. 38B). Interferon alpha production produced by RNA-LNPs may be dependent on the presence of MDA5, which detects HMW nucleic acid structures (FIG. 38B). In contrast, interferon alpha production was not abrogated with removal of the intracellular sensor RIG-I, which detects LMW nucleic acid structures (FIG. 38).

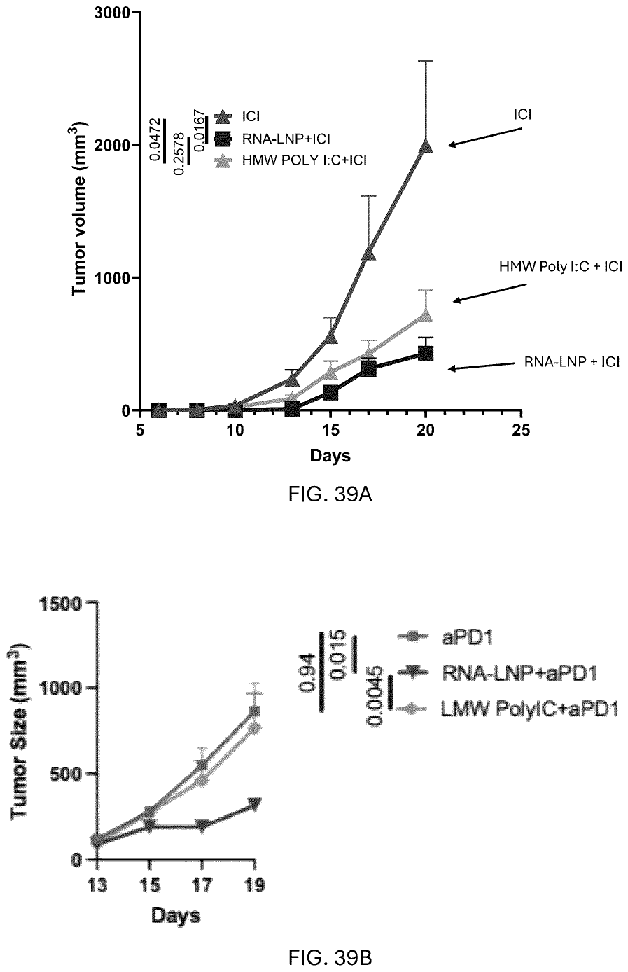

In addition, anti-tumor immunity was enhanced in response to HMW RNA structures. Mice with B16F0 tumors were treated with ICI with or without RNA-LNPs or high molecular weight (HMW) poly I:C starting on Day 3 (FIG. 39A). Here, HMW poly I:C provides similar inhibition of tumor growth relative to RNA-LNPs. In contrast, LMW poly I:C did not recapitulate this inhibition of tumor growth where Mice with B16F0 tumors were treated with ICI with or without RNA-LNPs or low molecular weight (LMW) poly I:C starting on Day 14 (FIG. 39B). Together, this data supports a role for HMW nucleic acid structures in mediating the anti-tumor effects of RNA-LNPs.

### Example 6: Expansion of Tumor-Reactive T Cells

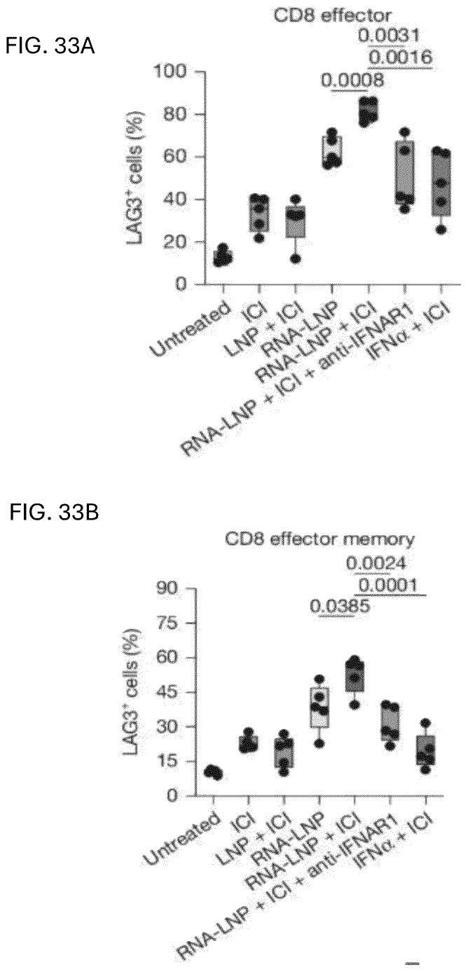

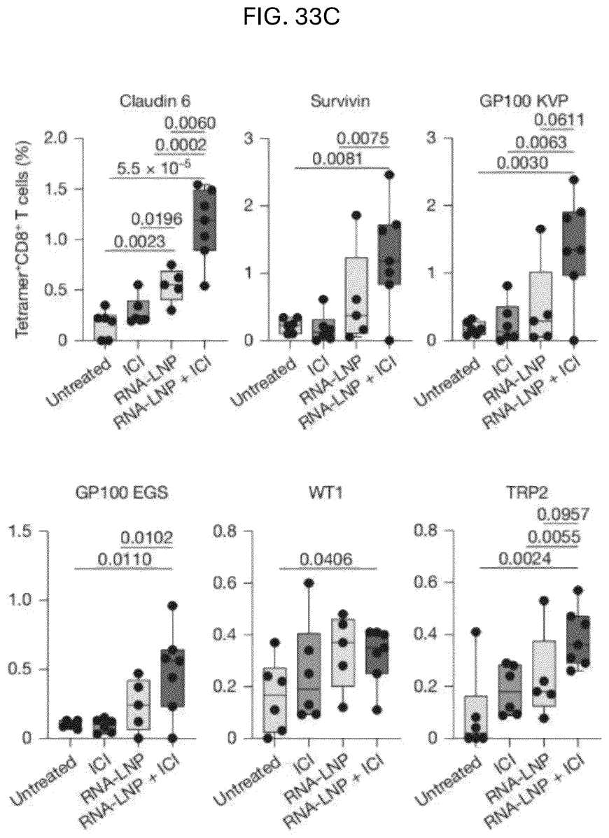

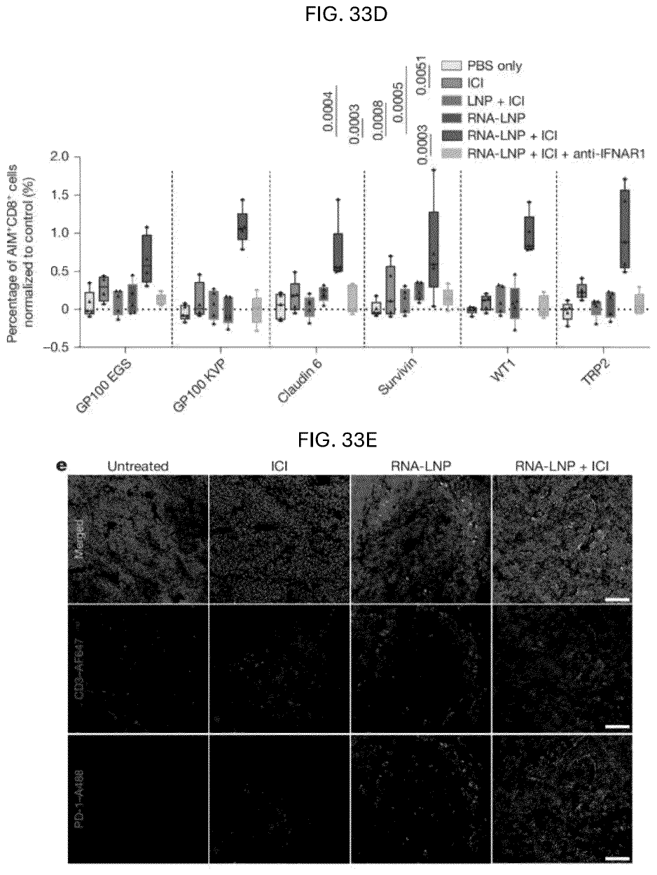

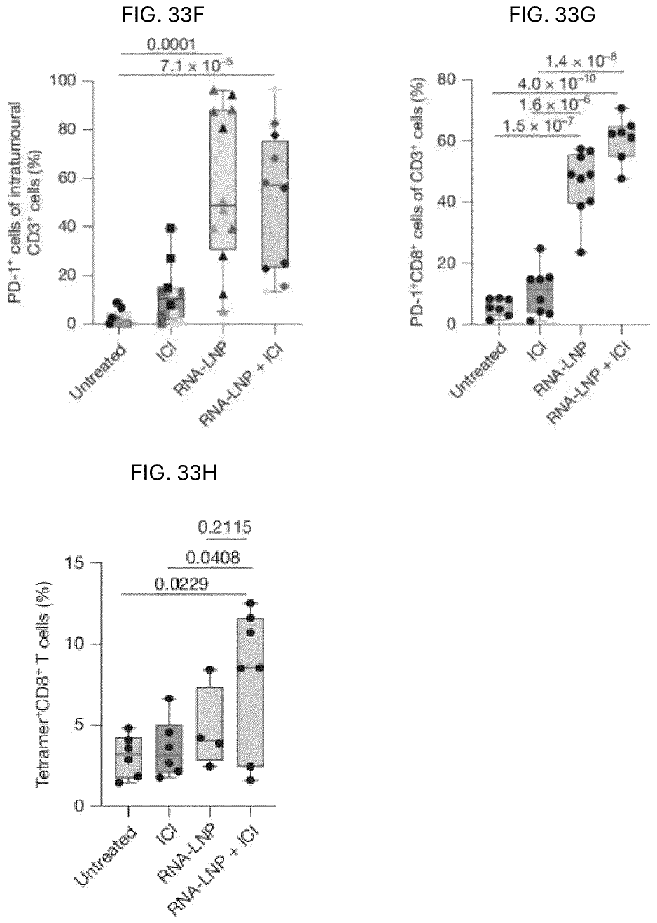

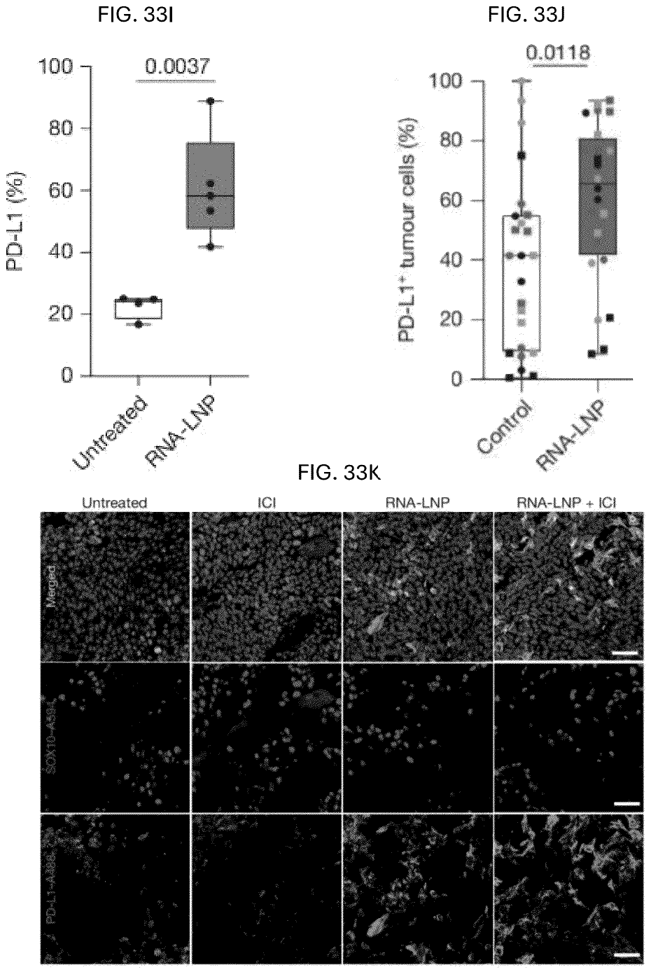

In addition to the precipitous activation of APCs, spike-encoding RNA-LNPs in combination with ICIs also elicited massive activation and expansion of effector and central memory CD8 T lymphocytes (FIGS. 33A-33B), highlighting the ability of these vaccines to rapidly prime the adaptive immune response and boost memory cell subsets. Concomitant with these findings, PD-1 expression was increased in T cells and effector/effector memory CD8+ cell subsets, underscoring the potent ability of combination therapy to rapidly prime T cells (data not shown).

While RNA-LNPs alone were sufficient for T cell activation characterized by co-expression of CD69, increased expression of PD-1 was only observed with the combination of RNA-LNPs and ICI (FIGS. 33C-33D).

Overall, these results illustrate the importance of systemic immunomodulation for recruitment of myeloid cells into lymphoreticular organs and the TME necessary for enhancing likelihood of tumor antigen presentation to cognate T cells. Once activated, T cells can penetrate TME but only survive and persist in the presence of ICIs.

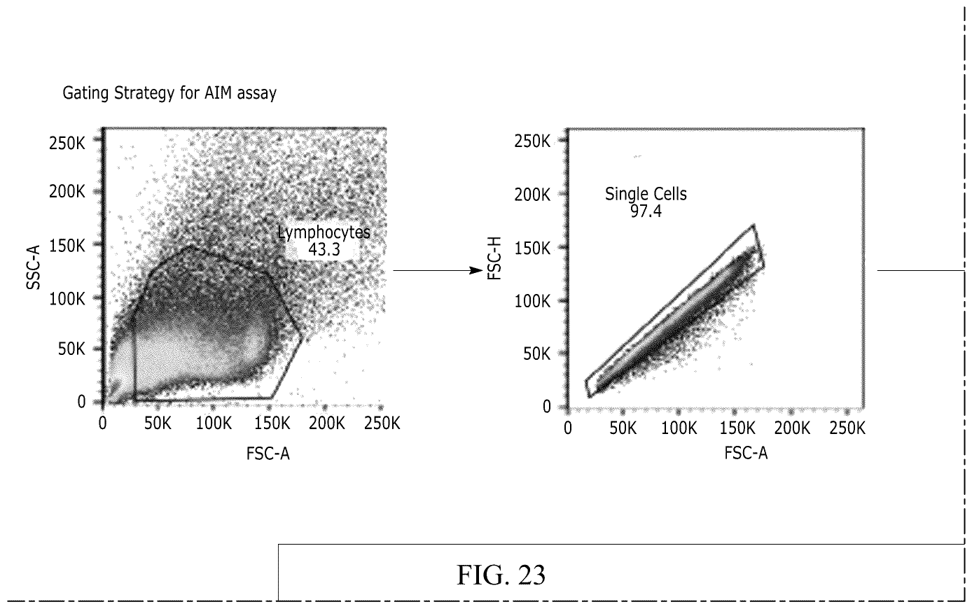

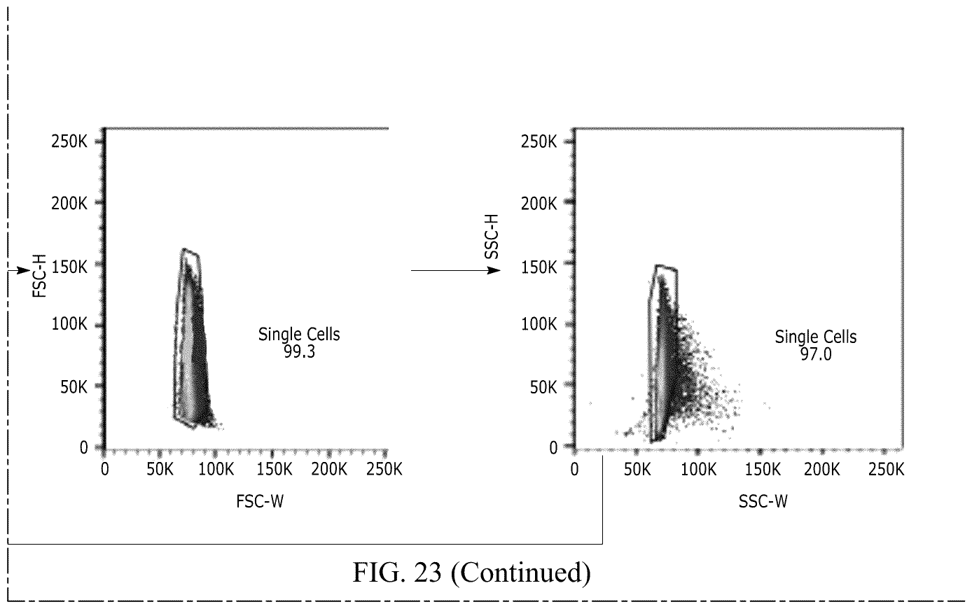

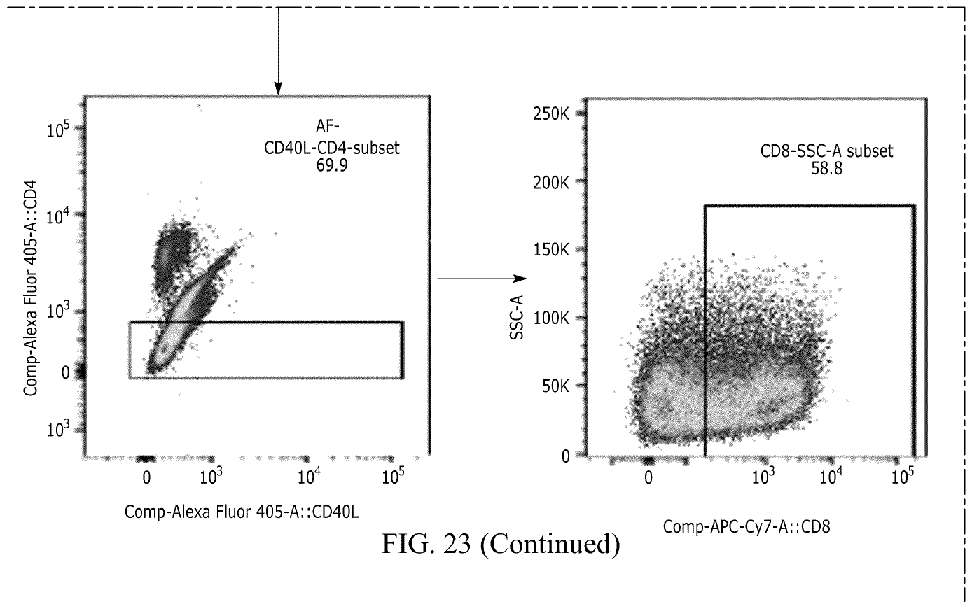

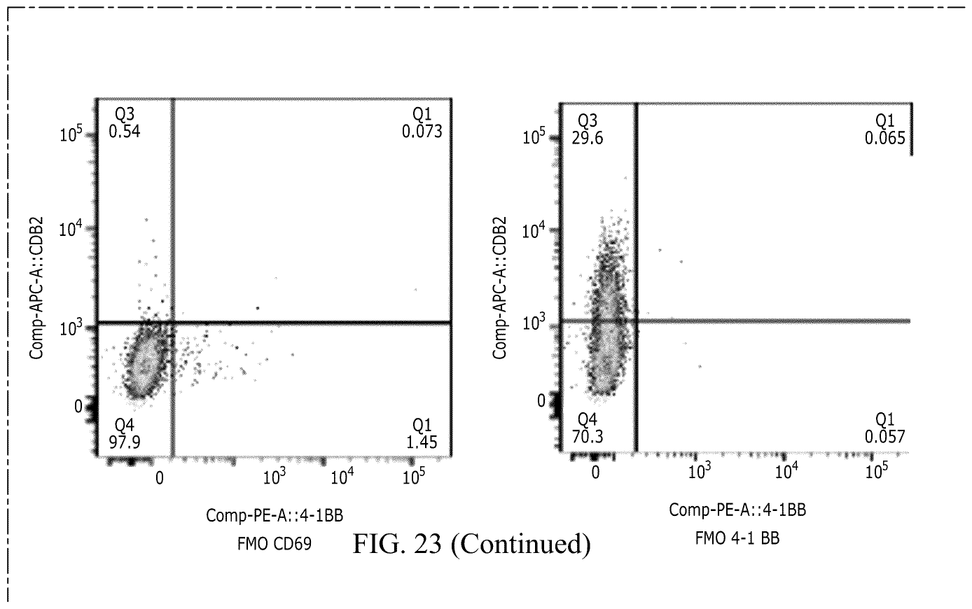

To confirm that spike RNA-LNPs were mediating expansion of tumor reactive T cells, CD8+ cells were sorted from spleens of treated mice and stained them with tetramers targeting peptides with high predicted binding affinity from six melanoma-associated antigens (GP-100 EGS, GP-100 KVP, claudin 6, survivin, WT1, and Trp2). It was found that combination therapy with RNA-LNPs and ICIs stimulates expansion of tetramer reactive T cells, suggesting that these T cells recognize tumor antigen (FIG. 33C). To confirm that these T cells were truly tumor-reactive, an activation inducible marker (AIM) assay was leveraged. In this assay, antigen reactive T cells cultured ex vivo with overlapping peptide pools from the same tumor antigens are identified through tandem expression of co-stimulatory molecules CD69 and 4-1BB (FIG. 33F or FIG. 23). With this approach, a substantial increase in AIM+ CD8 T cells was detected from animals treated with RNA-LNPs and ICI, further supporting tumor reactivity (FIG. 33D). These data confirm that spike mRNA LNPs prime the immune response for activation, presentation and recognition of tumor associated antigens in a manner that can be significantly expanded through concomitant treatment with ICI.

Next, the T cell compartment in treated tumors was evaluated. In mice with B16F0, a substantial infiltration of PD1+CD8+ T cells was identified by both immunofluorescence (FIGS. 33E-33F) and flow cytometry (FIG. 33G).

Importantly, it was found that treatment with RNA-LNPs and ICI increased PD1 expression on total CD8+ cells by greater than twenty fold (2.39% versus 51.363%, P<0.0001) (FIG. 33G), and that PD1+CD8+ T cells dominated the total CD3+ T cell compartment in treated mice while representing only a small minority of total CD3+ T cells in untreated mice (5.69% vs 60.57%, p<0.001) (FIG. 33G). The antigen specificity of these cells was evaluated. Since there were many fewer CD8 T cells in tumors compared to spleens after enriching for CD8+ cells, the six tetramers described above were pooled for a single pan-tetramer stain containing all six targets. With this approach, it was found that CD8+ TILs from mice treated with RNA-LNPs and ICIs were twice as likely to be tetramer reactive compared to non-RNA-LNP controls (3.10% versus 7.98%, P=0.0229) (FIG. 33H).

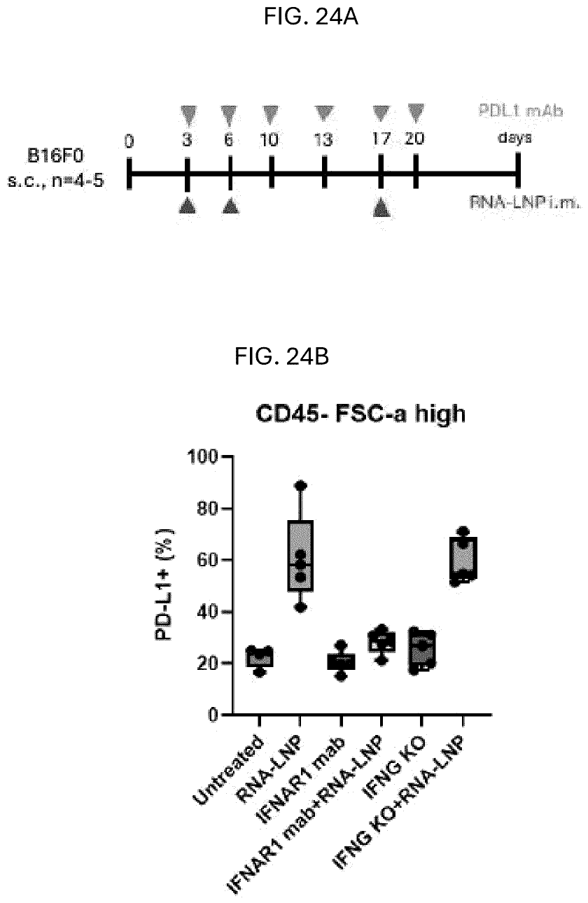

To confirm that these tumor-reactive cells recognized tumors, these tumor-reactive cells were evaluated for their direct impact on tumor cells. It was found that spike encoding RNA-LNPs, especially when combined with PD-L1 blockade, significantly increased PD-L1 expression on tumor cells (FIGS. 33J-33K). Blockade of interferon alpha signaling abrogated PD-L1 expression, confirming its importance in initiating immunotherapy response (FIGS. 24A-24B). Together, this data suggests that spike RNA-LNPs stimulate production and infiltration of activated, tumor-reactive CD8 T cells that can overcome compensatory expression of PD-1/PDL1 in the presence of ICI.

RNA-LNP vaccines stimulated tumor-specific T cell activation in murine models, promoting durable anti-tumor immunity. Preclinical experiments utilized murine B16F0 melanoma or genetically modified melanoma cells expressing ovalbumin (B16F10-OVA). Immune assays, including tetramer staining and activation-induced marker (AIM) assays, revealed enhanced effector and central memory CD8+ T cell expansion following vaccination (FIGS. 33A-33B).

Tumor-infiltrating lymphocytes (TILs) exhibited increased antigen specificity for melanoma-associated antigens (GP-100, Trp2) and elevated PD-1 expression (FIGS. 33C-33E). RNA-LNP vaccines amplified antigen presentation efficiency by APCs in lymph nodes and tumors, overcoming compensatory immune suppression mechanisms like PD-L1 upregulation (FIGS. 33H-33l).

### Example 7: Translational Validation in Human Subjects

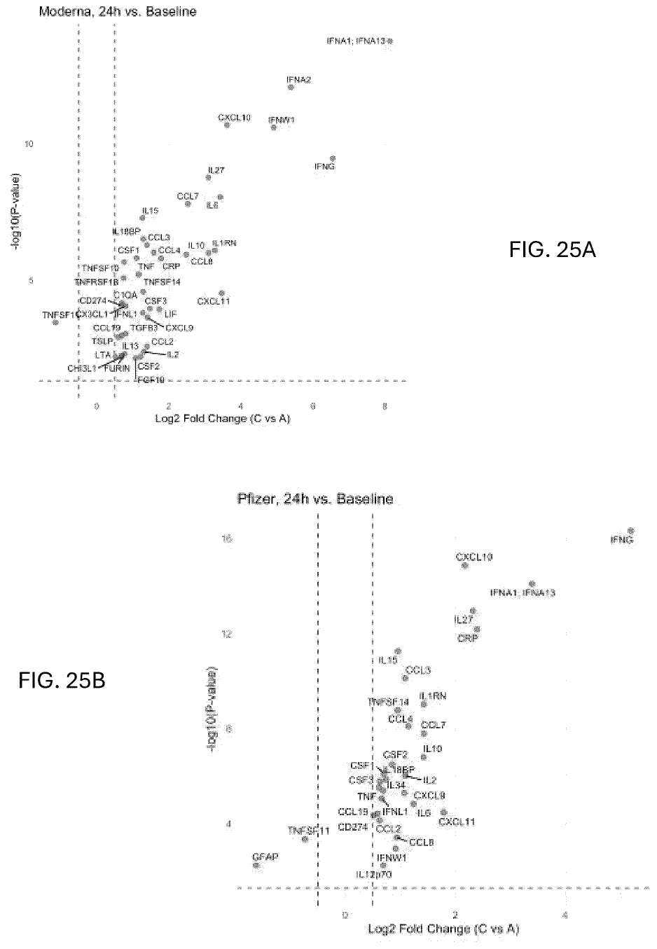

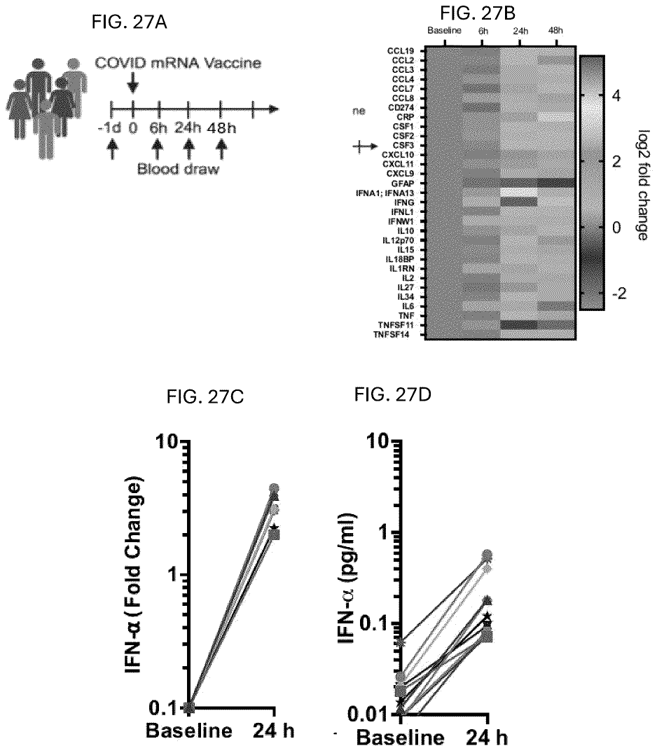

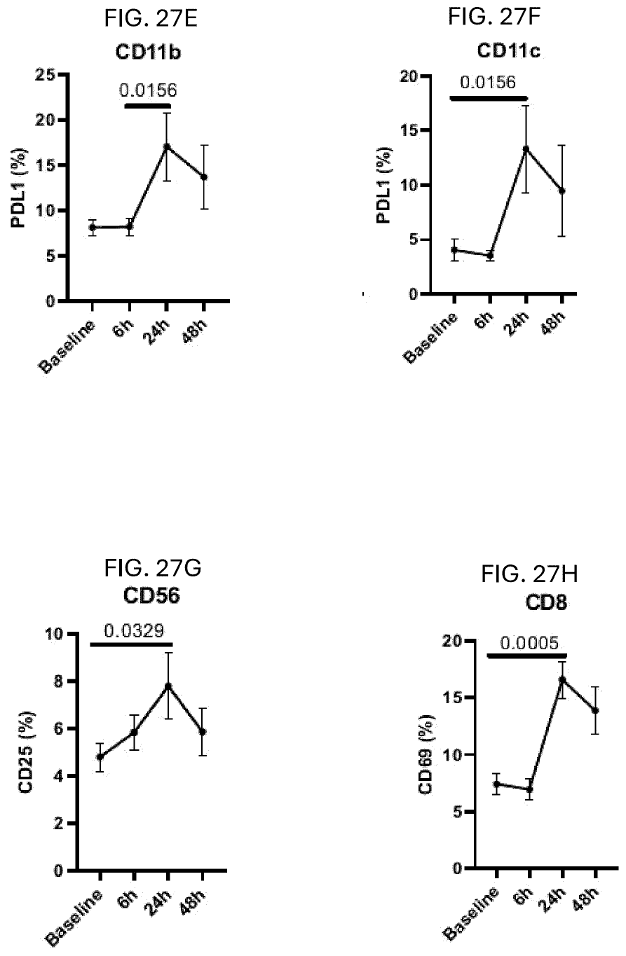

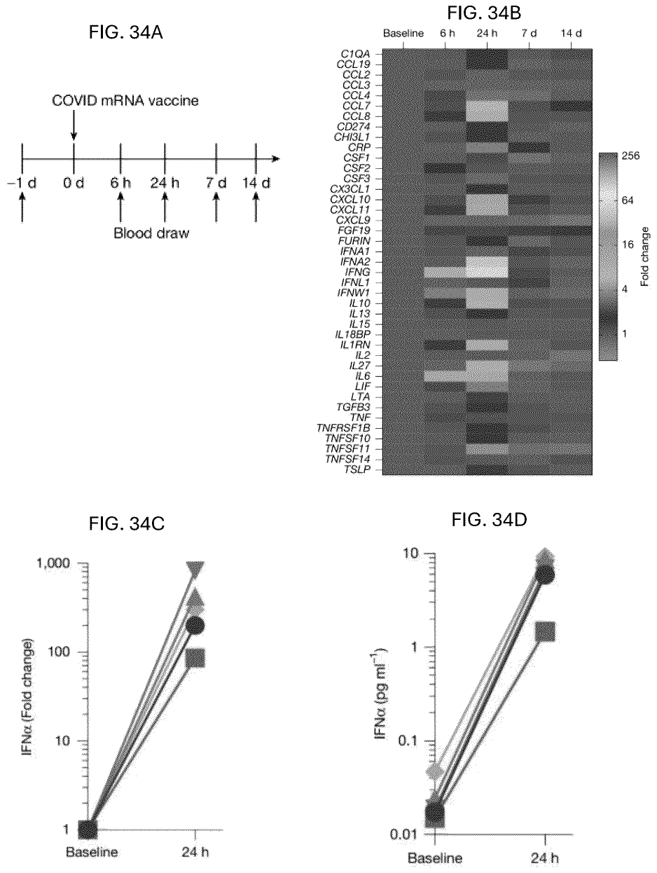

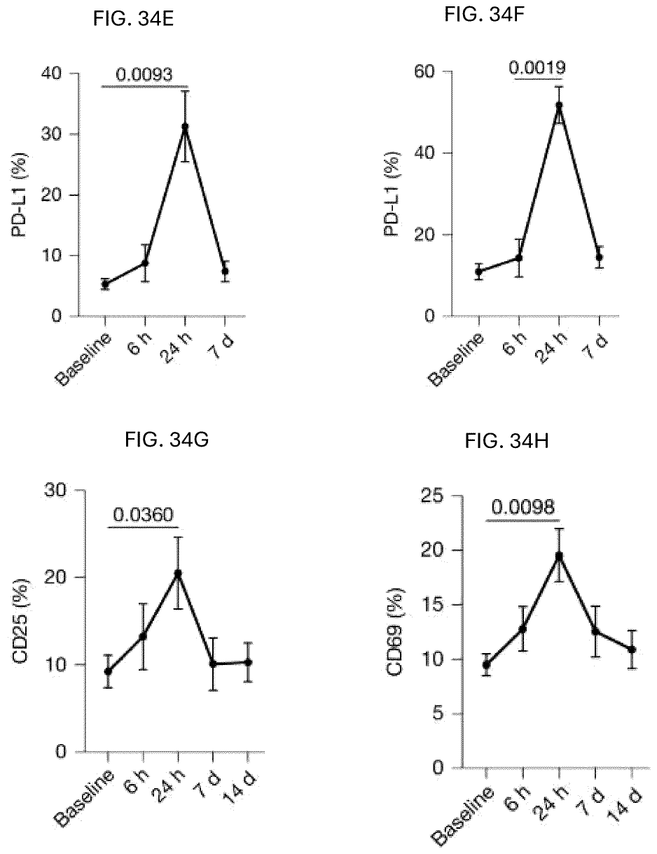

Since there are species-specific differences in how humans and mice respond to mRNA, it was important to confirm that the pathways identified in murine models were relevant in human systems. To do this, blood and serum samples were collected from five healthy volunteers at baseline and 6 hours, 24 hours, 7 days, and 14 days after receipt of mRNA-1273 Spikevax Monovalent XBB.1.5 (COVID mRNA Vaccine, 2023-2024 formulation, with 50 mcg mRNA) (FIG. 4A, FIG. 34A). Serum from these volunteers was evaluated with the NULISA-Seq Inflammation Panel (Alamar Biosciences), which is a multiplex assay designed to sensitively detect over 250 immune related cytokines. Interestingly, the only cytokines that were consistently elevated six hours after immunization were IL-6 and IFN-γ (FIG. 4B, FIG. 34B, or FIGS. 27A-27D). In contrast, a multitude of inflammatory cytokines surged at 24 hours after immunization, including IFN-α, IFN-γ, IFN-ω, IFN-γ-inducible protein-10 (CXCL10), CXCL11, and IL-27 (FIG. 4B, FIG. 34B, or FIGS. 25A-25B). Since IFN-α was found to be essential for antitumor activity in mice, there was particular interest in the kinetics of this protein. Interestingly, not only was IFN-α upregulated, but it was the most upregulated cytokine at any timepoint, increasing by an average of ˜280-fold relative to baseline to a final serum concentration between 1 and 10 pg/mL (FIG. 4C or FIG. 34C). Interestingly, although IL-1 was not found to be significantly increased at any timepoint, IL-1R antagonist (IL1RN) was significantly elevated at 24 hours, suggesting a compensatory response to a surge in IL-1 and IL-1R (FIG. 4B or FIG. 34D). As expected, cytokine responses to COVID mRNA vaccination were short-lived in healthy subjects, with all cytokines returning to baseline levels by 7 days (FIG. 4B or FIG. 34D).

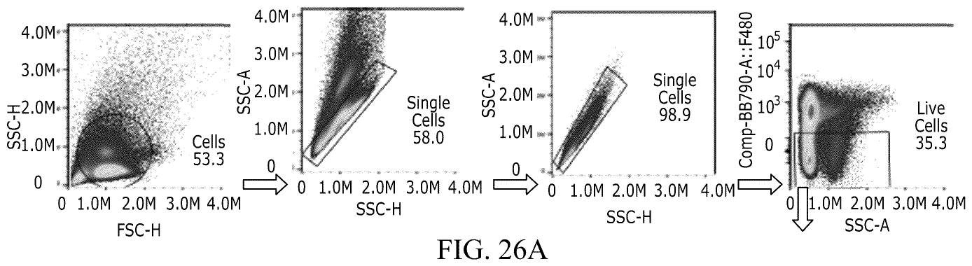

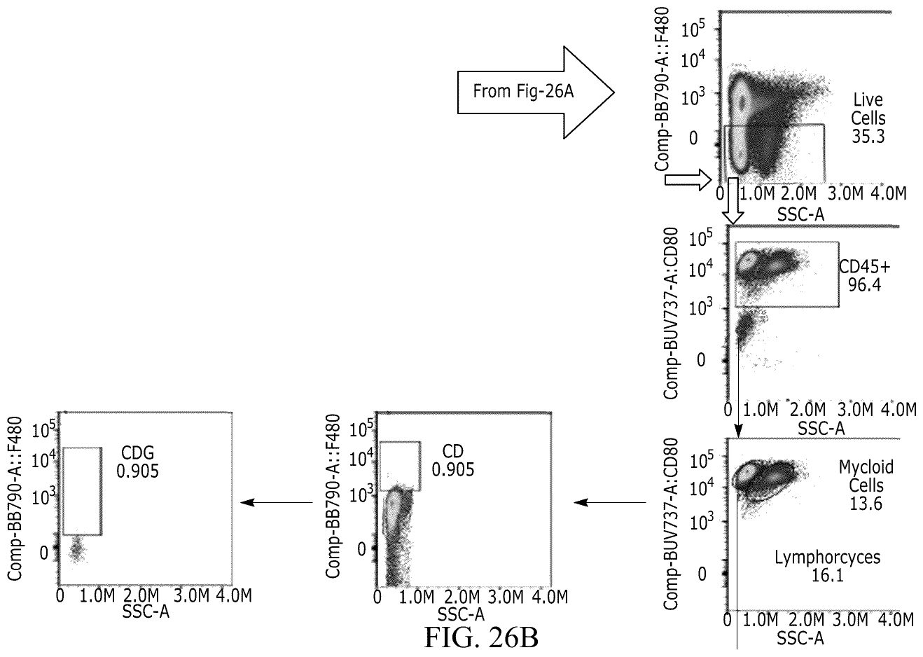

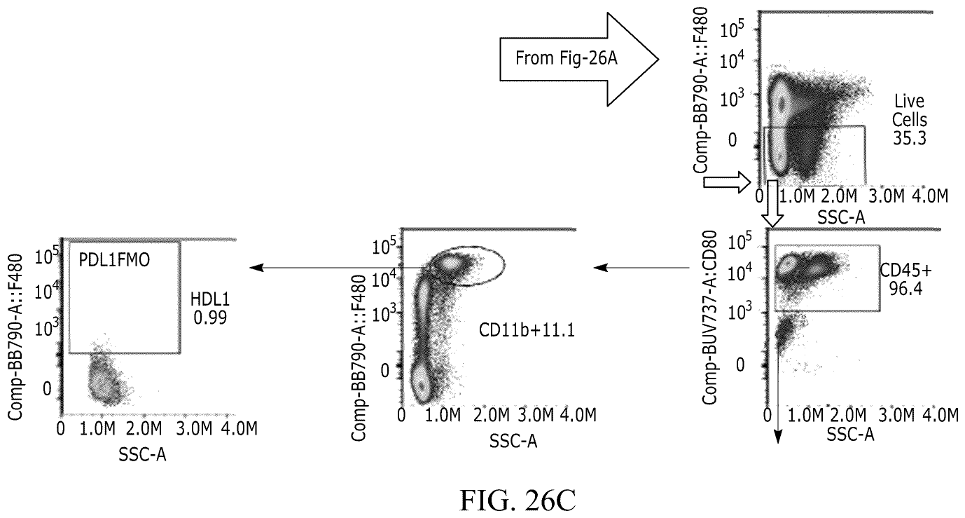

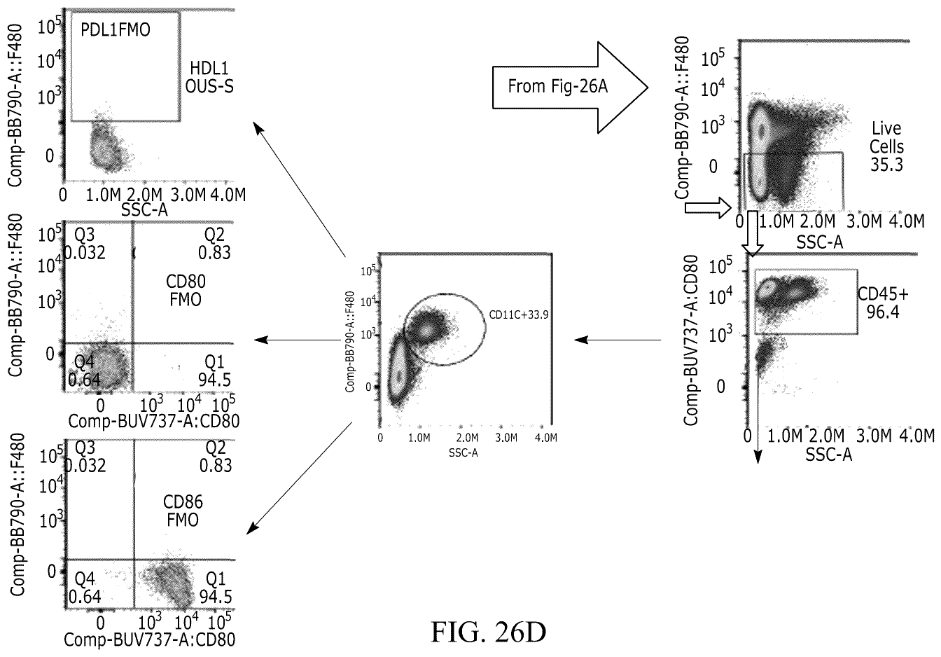

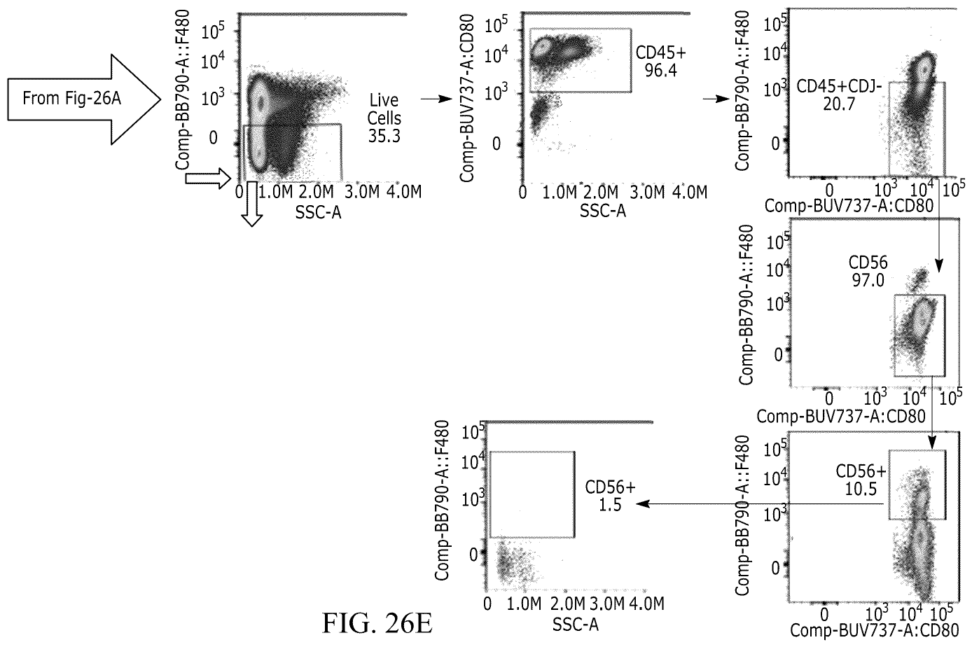

To evaluate the impact of COVID mRNA induced antiviral cytokines on immune cells, the phenotypes of circulating myeloid cells were evaluated by flow cytometry. Consistent with our findings in murine models, it was found that mRNA immunization drove innate immune activation exemplified by increased expression of PD-L1 on circulating CD11b+ myeloid cells and CD11c+ dendritic cells (FIGS. 4D-4E, FIG. 26, FIG. 34B, or FIG. 34E). Vaccination was also associated with activation of natural killer cells exemplified by a doubling of expression of IL-2-Ra (CD25) on CD56hi cells (FIG. 4F or FIG. 34F) and circulating T cells exhibited by a 30% increase in expression of CD69 (FIG. 4G or FIG. 34G). This T cell activation did not persist at the 7 or 14 days timepoint, which may be a result of the lack of target in these healthy subjects or lymphoreticular localization of activated T cells for polarization into memory cells.

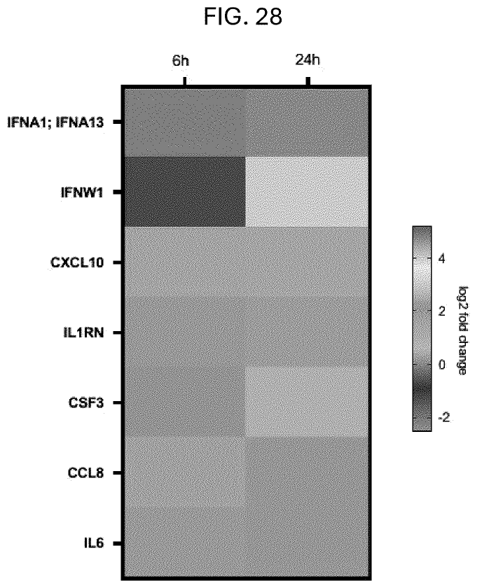

This study was repeated in eleven healthy volunteers who received COMIRNATY® (BNT162b2, COVID-19 mRNA Vaccine, 2024-2025 Formula, with 30 mcg mRNA) vaccine (FIGS. 27A-27H). Although very similar changes to the cytokine profile overall were found, the magnitude of the increase in type I interferon and innate immune activation by flow cytometry were significantly reduced with BNT162b2, which contains significantly less mRNA relative to mRNA-1273 (FIG. 28).

Healthy human subjects receiving SARS-CoV-2 mRNA vaccines exhibited immune profiles similar to those observed in murine models. Venipuncture blood samples were collected at baseline and multiple time points post-vaccination for analysis of cytokines and immune cell activation. The study involved SPIKEVAX® (mRNA-1273) and COMIRNATY® (BNT162b2) formulations.

Circulating levels of IFN-α increased up to 280-fold within 24 hours post-vaccination, alongside elevated Th1 chemokine expression (CXCL10, CXCL11), measured using multiplex cytokine panels (FIGS. 4B-4C or FIGS. 34B-34D). Flow cytometry revealed increased activation of myeloid cells, dendritic cells, and NK cells (FIGS. 4D-4F or FIGS. 34D-34F). T cell populations briefly exhibited CD69 activation markers, aligning immunological timelines with observations in murine studies.

Consistent with preclinical data, these responses declined rapidly within 7-14 days, demonstrating the transient but impactful immune priming associated with mRNA vaccines.

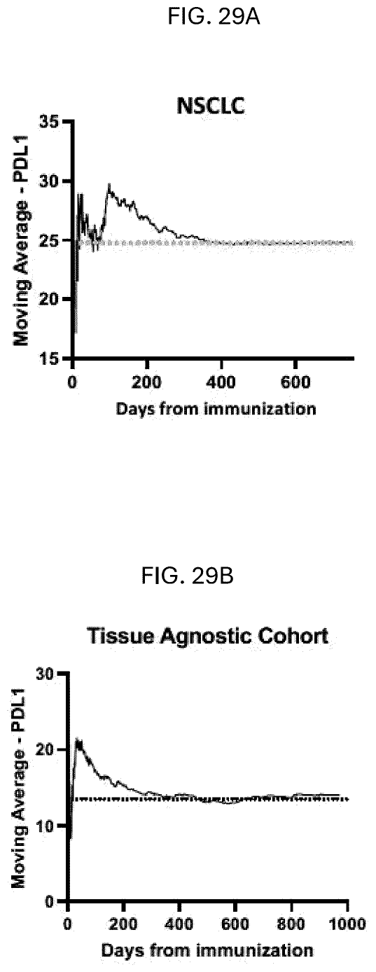

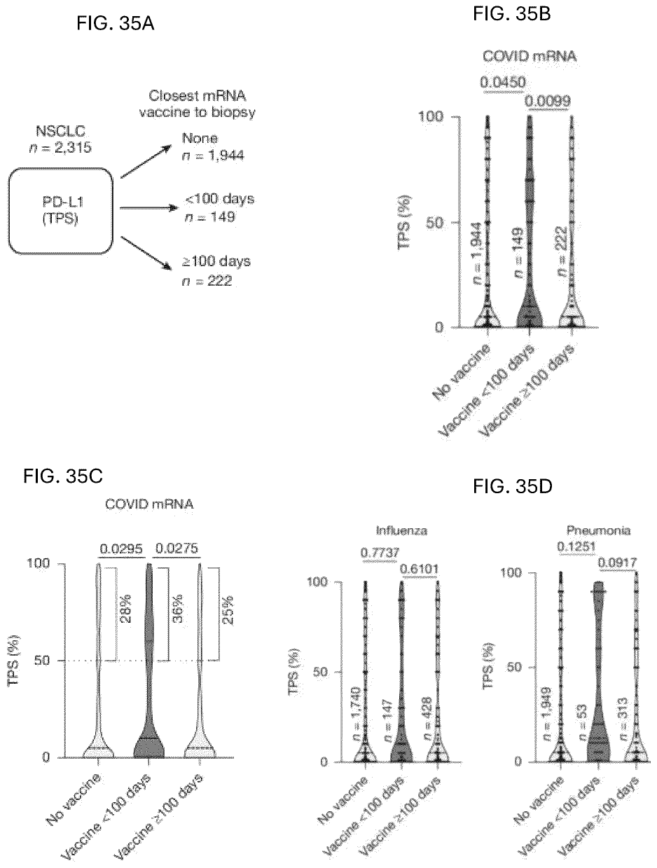

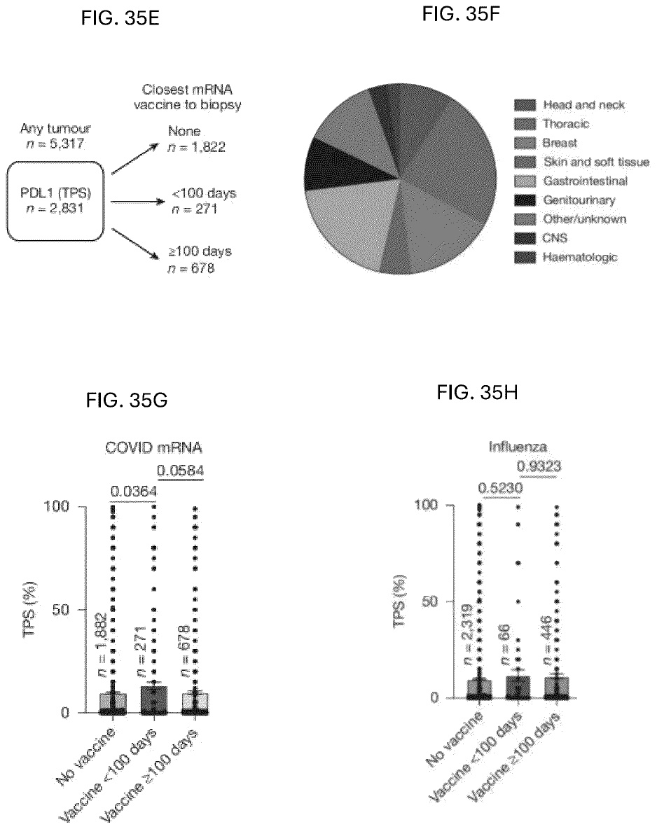

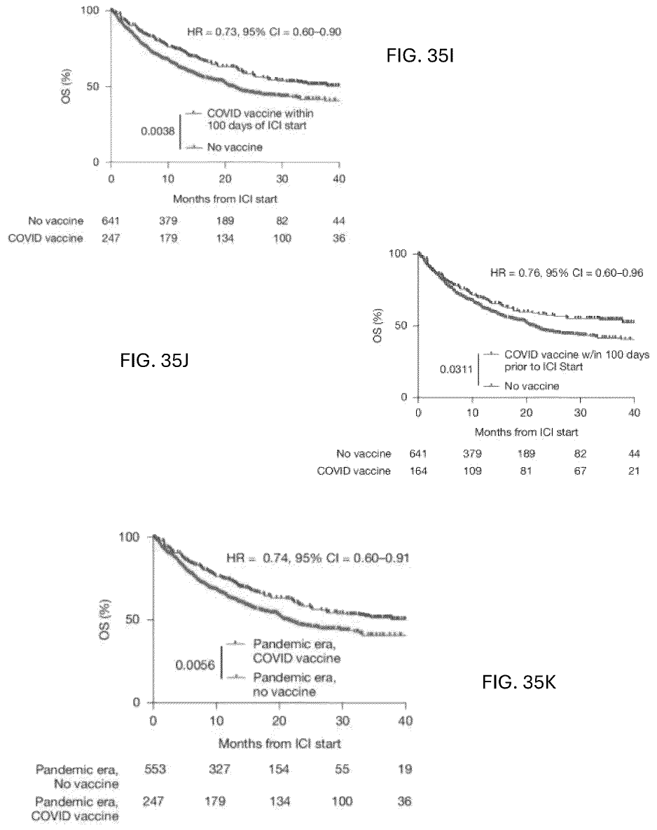

Since infiltration of antigen specific T cells in tumors is known to be associated with increases in PD-L1 expression on tumor cells and was correlated with antitumor response in our preclinical models, it was hypothesized that patients who received a COVID mRNA vaccine would exhibit higher PD-L1 expression on their tumors. To test this hypothesis, two cohorts of patients were assembled. First, 2,358 pathology reports were evaluated from patients with NSCLC with biopsies reporting tumor proportion score (TPS) August 2019 and November 2023, separating patients into three groups based on the timing between their biopsy and their most recent mRNA vaccine (FIG. 5A or FIG. 35A). It was found that patients who had received a COVID mRNA vaccine within 100 days of biopsy exhibited a 24% increase in mean tumor proportion score (TPS) of PD-L1 compared to patients who had not received any COVID mRNA vaccines prior to biopsy (31% vs 25%, p=0.0450) and a 41% increase in mean TPS relative to patients who received an mRNA vaccine more than 100 days prior to biopsy (31% vs 22%, P=0.0099) (FIG. 5B, FIGS. 29A-29B, or FIG. 35B). Since TPS of 50% is a clinically important threshold to determine whether patients with NSCLC are eligible for single agent immunotherapy instead of chemoimmunotherapy, these data were evaluated as a binary outcome around this threshold. Strikingly, it was found that patients who received a COVID mRNA vaccine were 29% more likely to exceed the 50% TPS threshold over unvaccinated patients (36% vs 28%, P=0.0295) (FIG. 5C or FIG. 35C), suggesting that mRNA vaccines have sufficient impact on TPS to modify treatment decisions. In contrast, pre-biopsy influenza and pneumonia vaccines were not associated with TPS changes (FIG. 5D or FIG. 35D).

To expand our findings beyond NSCLC and melanoma, a separate cohort of patients was assembled including all patients at a quaternary referral center with pathology reports including the term “PD-L1” in a four-year period including the pandemic era (FIG. 5E or FIG. 35E). Altogether, 5,317 unique pathology reports were identified with the term “PD-L1” including 2,231 reporting TPS from a diverse array of patients representing a variety of primary sites and histologies (FIG. 5F or FIG. 35F). In this cohort, receipt of a COVID mRNA vaccine within 100 days prior to biopsy was associated with a 37% increase in TPS (13.3% versus 9.7%, P=0.0364) (FIG. 5G or FIG. 35G), twice the effect seen in NSCLC patients. As in the NSCLC cohort, influenza vaccines were not associated with similar increases in TPS (FIG. 5H or FIG. 35H). The survival analysis was then repeated in this broad patient cohort, including all patients at our institution with a biopsy for PD-L1 in our dates of interest who received ICI (n=888). In this cohort, patients who received a COVID vaccine within 100 days of initiating ICI experienced significantly improved survival relative to their unvaccinated peers (P=0.0038, HR=0.73, 95% Cl=0.60-0.90) (FIG. 5I or FIG. 35I). This effect was consistent when limited to patients who received their vaccine within 100 days prior to the start of ICI (P=0.0311, HR=0.76, 95% Cl=0.60-0.96) (FIG. 5J or FIG. 35J) and patients who started their ICI in the pandemic era (P=0.0056, HR=0.74, 95% Cl=0.60-0.91) (FIG. 5K or FIG. 35K).

### Example 8: Preferential Benefit in “Cold” Tumors

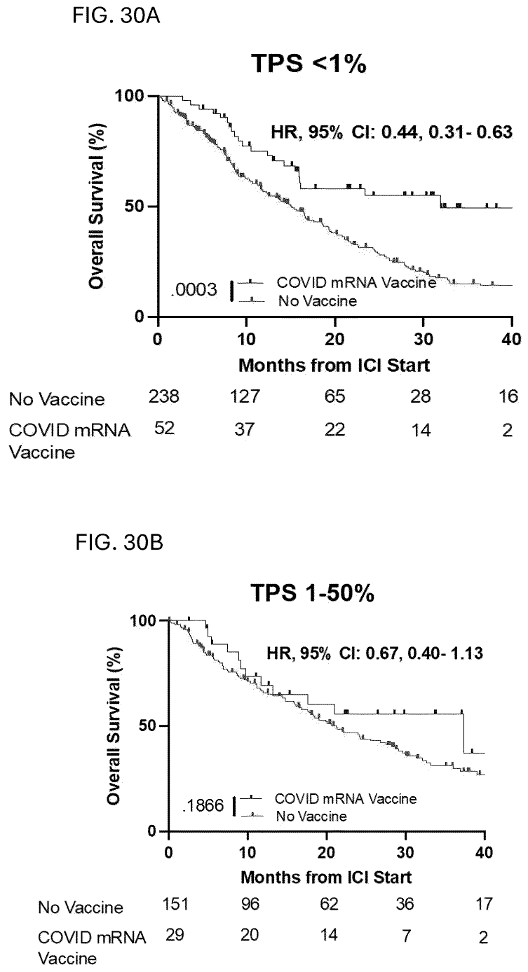

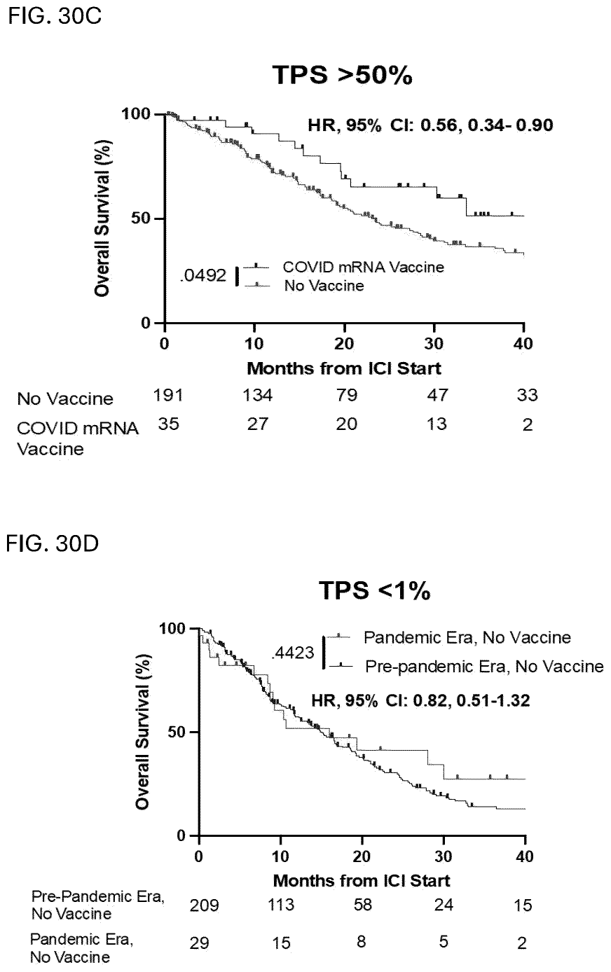

The impact of baseline TPS on vaccine sensitization was evaluated. Remarkably, it was determined that receipt of COVID mRNA vaccine within 100 days of initiating ICI had the greatest impact on overall survival for Stage IV NSCLC patients with TPS<1% relative to those patients with a TPS at biopsy of 1-49.9% or ≥50% (FIGS. 30A-30C). This effect was not explained by changes in patient management during the pandemic period, as Stage IV patients with TPS<1% at biopsy who did not receive a vaccine did not have significantly different outcomes before the pandemic as opposed to during the pandemic era (FIG. 30D).

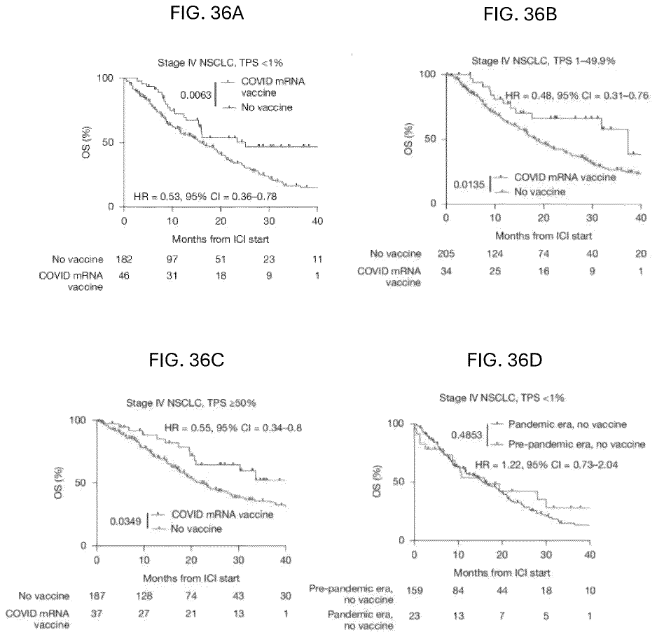

Next it was evaluated whether vaccination could restore immune sensitivity in patients with immunologically ‘cold’ tumors. Pre-vaccine TPS was used as a surrogate for immune sensitivity, as NSCLC patients with TPS<1% have reduced benefit from ICIs compared to patients with baseline TPS>1%. Among patients with stage IV NSCLC and baseline TPS<1%, those who received a COVID-19 mRNA vaccine within 100 days of initiating ICI exhibited OS similar to that of patients with baseline TPS>1%, suggesting restored sensitivity to ICIs (FIG. 30A or FIG. 36A). In addition, the association between COVID-19 mRNA vaccination and OS was similar for patients with TPS<1% relative to those patients with a TPS at biopsy of 1-49.9% or ≥50% (FIGS. 30A-30D or FIGS. 36A-36C). This effect was not explained by changes in patient management during the pandemic period, as patients with stage IV NSCLC with TPS<1% at biopsy who did not receive a vaccine had similar outcomes before and during the pandemic era (FIG. 36D).

Together, this data suggests a model in which mRNA vaccines targeting non-tumor related antigens stimulate robust antitumor immune responses (FIG. 10). mRNA vaccines first stimulate a surge in antiviral cytokines including IFN-α that drive systemic innate immune activation. Tumor resident innate immune cells activated by this cytokine surge prime T cells, which then secrete IFN-gamma in tumors that can evade response through compensatory expression of PD-L1. In combination with ICI, COVID mRNA vaccines prime systemic immune response and overcome compensatory intratumoral PD-L1 expression eliciting tumor regression and improved survival.

Retrospective data analysis highlighted preferential sensitization of immunologically “cold” tumors (low PD-L1 expression, TPS<1%) to ICIs following COVID mRNA vaccination. Using biopsy TPS data from NSCLC patients at MDACC, survival in vaccinated patients with cold tumors improved significantly compared to matched unvaccinated controls (FIGS. 30A-30C).

Stage IV NSCLC patients with TPS<1% exhibited survival outcomes comparable to those with TPS≥50%, reflective of enhanced tumor immune engagement (FIG. 30D). Propensity score matching balanced covariates such as primary tumor histology, baseline ECOG scores, and prior vaccine exposures, confirming the robustness of these findings.

This reinforces the therapeutic potential of RNA-LNP vaccines in addressing “cold” tumors historically insensitive to ICI therapy.

### Example 9: Benefits Associated with the Interval Between mRNA Vaccination and ICI Initiation

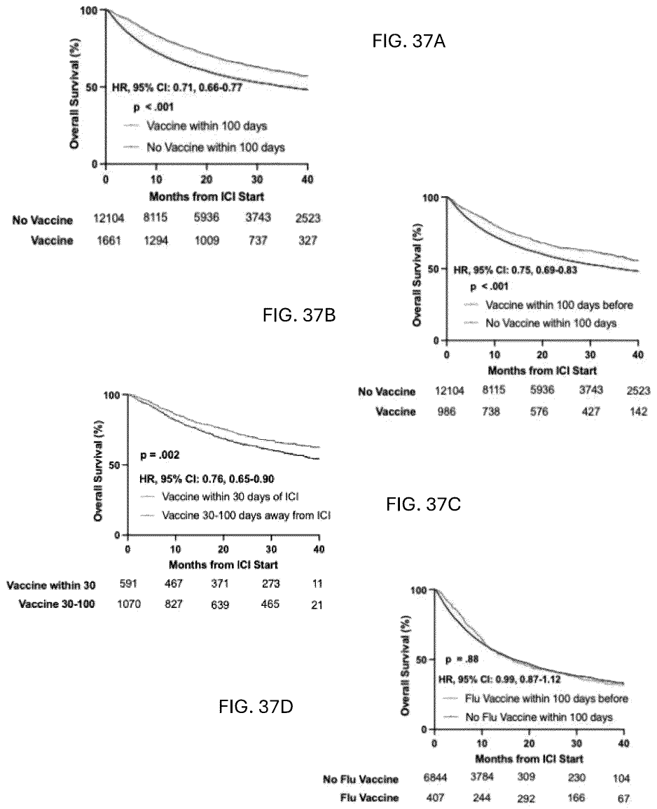

Interestingly, patients who received COVID mRNA vaccination within 100 days of ICI (n=1,661) also experienced significant survival benefits compared to all patients across the entire 13,765 patient cohort who did not receive a COVID mRNA vaccine within 100 days of ICI (n=12,104) (35.37 months vs. Undefined, logrank HR 0.71, 95% Cl 0.66-0.77, p<0.001) (FIG. 37A). This survival benefit was maintained when narrowing the window of inclusion to only those patients who received COVID mRNA vaccination in the 100 days prior to ICI as an additional control for immortal time bias (Undefined vs. 35.37 months, logrank HR 0.75, 95% Cl 0.69-0.83, p<0.001) (FIG. 37B). The association between vaccination and survival was also accentuated among patients who received a COVID mRNA vaccine within 30 days of initiating ICIs (n=591) compared to those who received their vaccines within 30-100 days of initiating ICIs (n=1070) (49.27 months vs. Undefined, logrank HR 0.76, 95% Cl 0.65-0.90, p=0.002) (FIG. 37C). In contrast, receipt of influenza vaccination within 100 days was not associated with improved OS among patients treated with ICI with no history of COVID mRNA vaccination (logrank HR: 0.99, 95% Cl: 0.87-1.12, p=0.88) (FIG. 37D).

Together, these data suggest a model in which mRNA vaccines targeting non-tumor-related antigens stimulate robust antitumor immune responses that sensitize tumors to ICIs. mRNA vaccines first stimulate a surge in antiviral cytokines, including IFNα, that drive systemic innate immune activation. Tumor-resident innate immune cells activated by this cytokine surge prime T cells, which become activated and infiltrate tumors. Although tumor cells evade attack by upregulating PD-L1 expression, combination with ICI enables COVID-19 mRNA vaccines to overcome this compensatory response, eliciting tumor regression and improved survival.

### Examples Discussion

Immunotherapy promises to deliver systemic anti-cancer therapy with long-term memory preventing recurrence. However, ICI relies on pre-existing anti-cancer immunity. Since most cancers are devoid of strong pre-existing anti-tumor immunity, ICIs often fail. mRNA vaccines have recently emerged as a promising strategy to generate anti-cancer immunity, with anti-tumor effects most magnified in combination with ICI. mRNA vaccines are generally thought to achieve anti-tumor immune responses primarily by directing tumors toward the antigens encoded by the mRNA. We hypothesized that the unique clinical success of mRNA therapeutics is buoyed by tumor remodeling initiated by the robust cytokine responses that they produce. Here, mRNA vaccines targeting the COVID spike protein were used to demonstrate that mRNA vaccines improve survival in combination with ICI even when the mRNA does not code for tumor antigens. Spike RNA-LNPs elicit body-wide APC activation and migration to lymphoid organs. In addition, this APC activation and the resulting reprogramming of the TME produces highly activated tumor specific T cells that mediate tumor regression. Together with case reports demonstrating spontaneous tumor regression after COVID mRNA vaccines, this data supports the use of mRNA vaccines as non-specific immune agonists to sensitize tumors to ICI by reprogramming systemic and intratumoral immunity.

We found that mRNA vaccines targeting tumor antigens exhibit superior efficacy relative to those targeted against non-tumor antigens, supporting development of neoantigen-based personalized vaccines. The dramatic effects of off-the-shelf mRNA therapeutics targeting infectious disease antigens highlight the potential utility of these therapeutics as non-specific innate immune modulators. Since personalized neoantigen vaccines require significant manufacturing time, off-the-shelf mRNA LNPs targeting tumor-associated or even infectious disease antigens may represent widely available, low-cost alternatives for patients waiting for personalized neoantigen vaccines or in settings where personalized neoantigen vaccines are not available.

Within the field of personalized mRNA vaccines, divergent strategies are also emerging regarding which arm of the immune system to prioritize. While some personalized cancer vaccines utilize local injection of mRNA with N1-methyl-pseudouridine to “silence” innate sensors and prioritize T cell activation, we prioritized maximization of the innate immune response with systemic injection of unmodified mRNA LNPs. Although the local injection of “silenced” mRNA LNPs does not aim to directly modify the tumor immune microenvironment, the data provided herein show that even local injection of immunologically “silenced” mRNA LNPs produces dramatic innate immune activation sufficient to reprogram TMEs and sensitize tumors to ICI. In addition, augmenting innate immune activation by replacing N1-methyl-pseudouridine-modified mRNA with unmodified mRNA further enhances the antitumor effects of this approach. Together, these data suggest that modulation of innate immunity alone may be sufficient to engender clinically significant improvements in survival in patients receiving ICI.

Altogether, the studies described herein demonstrate that a widely administered off-the-shelf mRNA therapeutic may augment responses to standard of care ICI treatment, and provide support for using other mRNA therapeutics to reset patient immune systems for enhanced response to ICIs.

### TABLES

TABLE 1
Demographics for NSCLC patients

| | | No Vaccine | | COVID mRNA Vaccine within | |
|---|---|---|---|---|---|
| | | (n = 704) | | Days of ICI Initiation (n = 180) | |
| Interval from COVID- | | | | −100 to +100 Days | |
| mRNA vaccine, days | | | | from start of ICI | |
| Gender | | | | | |
| | Male, % | 354 | (50.3%) | 88 | (48.9%) |
| Age at ICI start, median | | 64.0 | (26-89) | 65.7 | (30.7-89) |
| (range), years | | | | | |
| Histology | | | | | |
| | Adenocarcinoma | 614 | (87.2%) | 158 | (87.8%) |
| | Squamous cell | 71 | (10.1%) | 18 | (10.0%) |
| | carcinoma | | | | |
| | Other | 19 | (2.7%) | 4 | (2.2%) |
| Ethnicity | | | | | |
| | Caucasian | 569 | (80.8%) | 147 | (81.7%) |
| | Black | 55 | (7.8%) | 17 | (9.4%) |
| | Asian | 46 | (6.5%) | 9 | (5.0%) |
| | American | 6 | (0.9%) | 1 | (0.6%) |
| | Indian/Alaskan | | | | |
| | Native | | | | |
| | Native | 1 | (0.1%) | 0 | (0%) |
| | Hawaiian/Pacific | | | | |
| | Islander | | | | |
| | Unknown | 5 | (0.7%) | 3 | (1.7%) |
| | Other | 22 | (3.1%) | 3 | (1.7%) |
| Clinical Stage | | | | | |
| | Stage IIIA | 68 | (9.7%) | 24 | (13.3%) |
| | Stage IIIB | 57 | (8.1%) | 10 | (5.6%) |
| | Stage IIIC | 5 | (0.1%) | 6 | (3.33%) |
| | Stage IV | 574 | (81.5%) | 140 | (77.8%) |
| Mutations | EGFR | 128 | (18.2%) | 30 | (16.7%) |
| | HER2/ERB | 8 | (1.1%) | 3 | (1.7%) |
| | BRAF | 39 | (5.5%) | 9 | (5.0%) |
| | KRAS | 200 | (28.4%) | 60 | (33.3%) |
| | ALK | 19 | (2.7%) | 2 | (1.1%) |
| | PIK3CA | 60 | (8.5%) | 14 | (7.8%) |
| | MET | 102 | (14.5%) | 17 | (9.4%) |
| | CDKN2A | 42 | (6.0%) | 11 | (6.1%) |
| | STK11 | 83 | (11.8%) | 22 | (12.2%) |
| | P53 | 340 | (48.3%) | 77 | (42.8%) |
| | NF | 68 | (9.7%) | 16 | (8.9%) |
| | RET | 18 | (2.6%) | 0 | (0.0%) |
| | ROS1 | 11 | (1.6%) | 5 | (2.8%) |
| | AKT | 1 | (0.1%) | 3 | (1.7%) |
| ICI Drug | Pembrolizumab | 448 | (63.6%) | 122 | (67.8%) |
| | Nivolumab | 73 | (10.4%) | 5 | (2.8%) |
| | Durvalumab | 80 | (11.4%) | 30 | (16.7%) |
| | Atezolizumab | 43 | (6.1%) | 9 | (5.0%) |
| | Pembrolizumab + | 0 | (0.0%) | 1 | (0.6%) |
| | Ipilimumab | | | | |
| | Nivolumab + | 60 | (8.5%) | 13 | (7.2%) |

Ipilimumab

Table 2: Cox univariate proportional hazards regression analysis of factors influencing overall survival in non-small cell lung cancer patients. “COVID mRNA Vaccine” is defined as the receipt of a COVID mRNA vaccination within 100 days of the initiation of immune checkpoint inhibition (ICI). “Brain metastasis at ICI start” is a binary variable which explains whether a patient had at least one brain metastasis at the initiation of ICI. “Clinical stage” is a simplified variable describing whether a patient is Stage IIIa, IIIb, IIIc or Stage IV. “PDL1” describes tumor proportion score at time of primary tumor biopsy. “Steroid use within 1 month of ICI start” indicated whether or not a patient received a systemic steroid within 1 month before or after immunotherapy start date. “Cycle of immunotherapy” described whether the patient was ICI-naïve, had received ICI once before, or had received ICI multiple times. “EGFR”, “HER2”, “BRAF”, “KRAS”, “ALK”, “PIK3CA”, “MET”, “CDKN2A”, “STK11”, “P53”, “NF”, “RET”, and “ROS1” are descriptions of whether patients had mutations in these genes at the time of tumor biopsy. Performance status quantified by the Eastern Cooperative Oncology Group performance status scale (ECOG), “Age at ICI start”, and comorbidities (heart disease, immunodeficiency, diabetes, other primary, etc) represent patient characteristics at the start of ICI. “Radiation immediately before ICI” describes whether or not a patient received radiation within 90 days before ICI”, while “Radiation with ICI” describes receipt of radiation within 60 days after the start of ICI. “Surgery immediately before ICI” describes any surgery prior to the initiation of ICI. “Gender” was described as a binary variable between 0, Female and 1, Male. “Race” was described as a binary variable between White, 1, and other, 0 in this dataset, due to low quantities of non-white populations. *p<0.05, **p<0.01, ***p<0.001 unless otherwise noted.

TABLE 2

| | | | | | | | P value |
|---|---|---|---|---|---|---|---|
| Variable | Beta | St. Error | HR | 2.5% CI | 97.5% CI | P value | [symbol] |
| COVID mRNA vaccination within 100 days of | −0.6324 | 0.1366 | 0.531 [symbol] | 0.4065 | 0.6944 | 3.63E−6 | **** |
| [symbol] | | | | | | | |
| Ethnicity American Indian/Alaskan Native | 0.2774 | 0.5030 | 1.319 [symbol] | 0.4923 | 3.5370 | 0.5814 | ns |
| [symbol] | | | | | | | |
| Ethnicity Asian (Reference: White) | 0.2327 | 0.1815 | 1.262 [symbol] | 0.8842 | 1.8012 | 0.1999 | ns |
| Ethnicity Black (Reference: White) | 0.1916 | 0.1609 | 1.211 [symbol] | 0.8835 | 1.6603 | 0.2339 | ns |
| Ethnicity Native Hawaiian/Pacific Islander | [symbol] | 1143.584 [symbol] | 0.000 [symbol] | 0.0000 | Inf | 0.9917 | ns |
| [symbol] | | | | | | | |
| Ethnicity Other (Reference: White) | 0.4646 | 0.2414 | 1.591 [symbol] | 0.9915 | 2.5544 | 0.0543 | ns |
| Ethnicity Unknown (Reference: White) | −0.5356 | 0.5798 | 0.585 [symbol] | 0.1879 | 1.8237 | 0.3557 | ns |
| Histology Other (reference: Adenocarcinoma) | −0.1513 | 0.3054 | 0.859 [symbol] | 0.4723 | 1.5642 | 0.6204 | ns |
| Histology Squamous (reference: Adenocarcinoma) | −0.0460 | 0.1568 | 0.955 [symbol] | 0.7024 | 1.2986 | 0.7693 | ns |
| Steroid use within 1 month of ICI Start | 0.3376 | 0.1186 | 1.401 [symbol] | 1.1108 | 1.7683 | 0.0044 | ** |
| Steroid use within 1 month of Vaccination | 0.6767 | 0.3470 | 1.967 [symbol] | 0.9965 | 3.8841 | 0.0512 | ns |
| Age at ICI Start | 0.0028 | 0.0042 | 1.002 [symbol] | 0.9946 | 1.0112 | 0.5019 | ns |
| ECOG | 0.4395 | 0.0585 | 1.552 [symbol] | 1.3840 | 1.7404 | 5.53E−14 | **** |
| PDL1 (TPS) | −0.0054 | 0.0014 | 0.994 [symbol] | 0.9920 | 0.9973 | 7.22E−5 | *** |
| Line of Immunotherapy Treatment | −0.1726 | 0.1241 | 0.841 [symbol] | 0.6597 | 1.0733 | 0.1644 | ns |
| EGFR | 0.2139 | 0.1132 | 1.238 [symbol] | 0.9921 | 1.5461 | 0.0587 | ns |
| KRAS | 0.1969 | 0.0966 | 1.217 [symbol] | 1.0075 | 1.4715 | 0.0417 | * |
| HER2ERBB2 | −0.0564 | 0.4498 | 0.945 [symbol] | 0.3914 | 2.2823 | 0.9003 | ns |
| BRAF | −0.2528 | 0.2096 | 0.776 [symbol] | 0.5150 | 1.1713 | 0.2279 | ns |
| AKT | −0.1137 | 0.7088 | 0.892 [symbol] | 0.2225 | 3.5802 | 0.8725 | ns |
| ALK | 0.3291 | 0.2714 | 1.389 [symbol] | 0.8164 | 2.3654 | 0.2253 | ns |
| MET | 0.1712 | 0.1244 | 1.186 [symbol] | 0.9299 | 1.5145 | 0.1688 | ns |
| STK11 | 0.6580 | 0.1261 | 1.930 [symbol] | 1.5080 | 2.4725 | 1.83E−7 | **** |
| ROS1 | −0.1977 | 0.3566 | 0.820 [symbol] | 0.4079 | 1.6508 | 0.5793 | ns |
| PIK3CA | 0.0685 | 0.1599 | 1.070 [symbol] | 0.7828 | 1.4650 | 0.6685 | ns |
| CDKN2A | 0.2217 | 0.1780 | 1.248 [symbol] | 0.8805 | 1.7693 | 0.2131 | ns |
| P53 | 0.1384 | 0.0912 | 1.148 [symbol] | 0.9604 | 1.3732 | 0.1292 | ns |
| NF | 0.2767 | 0.1403 | 1.318 [symbol] | 1.0017 | 1.7360 | 0.0486 | * |
| RET | 0.6564 | 0.2715 | 1.927 [symbol] | 1.1324 | 3.2820 | 0.0156 | * |
| Stage IIIb (reference: IIIa) | −0.0696 | 0.2636 | 0.932 [symbol] | 0.5564 | 1.5635 | 0.7916 | ns |
| Stage IIIc (reference: IIIa) | 0.0230 | 0.5287 | 1.023 [symbol] | 0.3630 | 2.8844 | 0.9653 | ns |
| Stage IV (reference: IIIa) | 0.7305 | 0.1785 | 2.076 [symbol] | 1.4634 | 2.9454 | 4.25E−5 | **** |
| English as Primary Language (Reference: No) | −0.0581 | 0.2285 | 0.943 [symbol] | 0.6030 | 1.4766 | 0.7993 | ns |
| Heart Disease | −0.1171 | 0.0913 | 0.889 [symbol] | 0.7437 | 1.0638 | 0.1997 | ns |
| Respiratory Condition | 0.5354 | 0.1215 | 1.708 [symbol] | 1.3460 | 2.1675 | 1.06E−5 | **** |
| Immune Deficiency | −0.0415 | 0.1551 | 0.959 [symbol] | 0.7078 | 1.3002 | 0.7891 | ns |
| Diabetes | 0.1742 | 0.1075 | 1.190 [symbol] | 0.9642 | 1.4695 | 0.1050 | ns |
| CKD | −0.0543 | 0.1294 | 0.947 [symbol] | 0.7350 | 1.2205 | 0.6747 | ns |
| Liver Failure | 0.2652 | 0.3568 | 1.303 [symbol] | 0.6479 | 2.6235 | 0.4572 | ns |
| CNS Disease | −0.8233 | 0.7087 | 0.439 [symbol] | 0.1094 | 1.7607 | 0.2453 | ns |
| Concurrent Chemotherapy during ICI | 0.2532 | 0.0917 | 1.288 [symbol] | 1.0761 | 1.5418 | 0.0058 | ** |
| Immunotherapy Agent Atezolizumab (Reference: | −0.0605 | 0.1973 | 0.941 [symbol] | 0.6394 | 1.3858 | 0.7593 | ns |
| [symbol] | | | | | | | |
| Immunotherapy Agent Durvalumab (Reference: | −0.6195 | 0.1681 | 0.538 [symbol] | 0.3872 | 0.7482 | 0.0002 | *** |
| [symbol] | | | | | | | |
| Immunotherapy Agent Nivolumab (Reference: | 0.2199 | 0.1403 | 1.245 [symbol] | 0.9464 | 1.6402 | 0.1170 | ns |
| [symbol] | | | | | | | |
| Immunotherapy Agent Nivolumab + | −0.0201 | 0.1627 | 0.980 [symbol] | 0.7124 | 1.3483 | 0.9015 | ns |
| Ipilimumab (Reference: | | | | | | | |
| [symbol] | | | | | | | |
| Immunotherapy Agent Pembrolizumab + | [symbol] | 772.5062 | 0.000 [symbol] | 0.0000 | Inf | 0.9865 | ns |
| [symbol] | | | | | | | |
| BMI | 0.0076 | 0.0116 | 1.007 [symbol] | 0.9850 | 1.0308 | 0.5124 | ns |
| Gender (Reference: Female) | 0.1582 | 0.0910 | 1.171 [symbol] | 0.9801 | 1.4001 | 0.0821 | ns |
| Previous history of malignancy at ICI Start | −0.0561 | 0.2714 | 0.945 [symbol] | 0.5555 | 1.6093 | 0.8364 | ns |
| Previous cycles of systemic therapy | 0.0951 | 0.0385 | 1.099 [symbol] | 1.0199 | 1.1859 | 0.0135 | * |
| Treatment Year | −0.0845 | 0.0275 | 0.919 [symbol] | 0.8707 | 0.9699 | 0.0022 | ** |

[symbol]

indicates data missing or illegible when filed

Table 3: Cox multivariate proportional hazards regression analysis of factors influencing overall survival in non-small cell lung cancer patients. This multivariate analysis included the factors in the univariate analysis that had a significant relationship with overall survival alone in the patient population. See Table 2 for descriptions of variables included in analysis. *p<0.05, **p<0.01, ***p<0.001 unless otherwise noted.

TABLE 3

| | | St. | | | | | P value |
|---|---|---|---|---|---|---|---|
| Variable | Beta | Error | adj HR | 2.5% CI | 97.5% CI | p value | interpret [symbol] |
| COVID mRNA vaccination within 100 days of | −0.6689 | 0.1671 | 0.5123 | 0.3692 | 0.7107 | 6.24E−5 | **** |
| [symbol] | | | | | | | |
| Steroid use within 1 month of ICI Start | 0.3289 | 0.1224 | 1.3895 | 1.0931 | 1.7663 | 0.0072 | ** |
| ECOG | 0.4291 | 0.0615 | 1.5358 | 1.3614 | 1.7326 | 3.06E− [symbol] | **** |
| PDL1 | −0.0047 | 0.0016 | 0.9953 | 0.9923 | 0.9983 | 0.0025 | ** |
| KRAS | 0.0308 | 0.1062 | 1.0313 | 0.8375 | 1.2700 | 0.7716 | ns |
| STK11 | 0.4524 | 0.1419 | 1.5720 | 1.1904 | 2.0760 | 0.0014 | ** |
| Stage IIIb (reference: IIIa) | −0.0020 | 0.2782 | 0.9980 | 0.5785 | 1.7217 | 0.9942 | ns |
| Stage IIIc (reference: IIIa) | 0.3138 | 0.5379 | 1.3687 | 0.4770 | 3.9275 | 0.5596 | ns |
| Stage IV (reference: IIIa) | 0.7516 | 0.2265 | 2.1204 | 1.3604 | 3.3051 | 0.0009 | *** |
| NF | 0.1835 | 0.1461 | 1.2014 | 0.9022 | 1.5999 | 0.2093 | ns |
| RET | 0.6024 | 0.2816 | 1.8265 | 1.0517 | 3.1721 | 0.0324 | * |
| Respiratory Condition | 0.3773 | 0.1265 | 1.4584 | 1.1381 | 1.8689 | 0.0029 | ** |
| Concurrent Chemotherapy during ICI | 0.1858 | 0.1261 | 1.2042 | 0.9405 | 1.5419 | 0.1406 | ns |
| Immunotherapy Agent Atezolizumab (Reference: [symbol] | −0.0345 | 0.2072 | 0.9661 | 0.6436 | 1.4500 | 0.8676 | ns |
| Immunotherapy Agent Durvalumab (Reference: [symbol] | 0.0563 | 0.2437 | 1.0579 | 0.6562 | 1.7056 | 0.8173 | ns |
| Immunotherapy Agent Nivolumab (Reference: [symbol] | 0.1373 | 0.1745 | 1.1472 | 0.8148 | 1.6150 | 0.4315 | ns |
| Immunotherapy Agent Nivolumab + Ipilimumab | 0.0069 | 0.1986 | 1.0070 | 0.6822 | 1.4863 | 0.9721 | ns |
| [symbol] | | | | | | | |
| Immunotherapy Agent Pembrolizumab + I [symbol] | −12.5047 | [symbol] | 0.0000 | 0.0000 | Inf | 0.9879 | ns |
| Previous cycles of systemic therapy | 0.1184 | 0.0412 | 1.1256 | 1.0383 | 1.2204 | 0.0041 | ** |
| Treatment Year | 0.0335 | 0.0387 | 1.0341 | 0.9586 | 1.1156 | 0.3862 | ns |

[symbol]

indicates data missing or illegible when filed

TABLE 4
Demographics for melanoma patients

| | | No Vaccine | | COVID mRNA Vaccine within | |
|---|---|---|---|---|---|
| | | (n = 167) | | Days of ICI Initiation (n = 43) | |
| Interval from COVID- | | | | −100 to +100 Days | |
| mRNA vaccine, days | | | | from start of ICI | |
| Gender | | | | | |
| | Male, % | 109 | (65.3%) | 30 | (69.8%) |
| Age at ICI start, | | 63.5 | (26.2-89) | 68.9 | (22.9-84.5) |
| median (range), years | | | | | |
| Ethnicity | | | | | |
| | Caucasian | 149 | (89.2%) | 40 | (93%) |
| | Black | 3 | (1.8%) | 0 | (0%) |
| | Asian | 2 | (1.2%) | 0 | (0%) |
| | Hispanic/Latino | 6 | (3.6%) | 3 | (7%) |
| | Unknown | 6 | (3.6%) | 0 | (0.0%) |
| | Other | 1 | (0.6%) | 0 | (0.0%) |
| Mutations | BRAF | 64 | (20.4%) | 15 | (34.9%) |
| | KIT | 15 | (9.0%) | 5 | (11.6%) |
| | PTEN | 16 | (9.6%) | 2 | (4.7%) |
| | NRAS | 34 | (20.4%) | 9 | (20.9%) |
| | GNA11 | 2 | (1.2%) | 2 | (4.7%) |
| | GNAQ | 2 | (1.2%) | 1 | (2.3%) |
| ICI Drug | Pembrolizumab | 20 | (12%) | 8 | (18.6%) |
| | Nivolumab | 61 | (36.5%) | 14 | (32.6%) |
| | Avelumab | 1 | (0.6%) | 0 | (0%) |
| | Ipilimumab + | 80 | (47.9%) | 19 | (44.2%) |
| | Nivolumab | | | | |
| | Ipilimumab + | 4 | (2.4%) | 2 | (4.7%) |
| | Pembrolizumab | | | | |
| | Ipilimumab | 1 | (0.6%) | 0 | (0%) |

TABLE 5
T cell panel information (Antibody)

| Marker | Color | Clone | Company | Cat# | ul/100 μL |
|---|---|---|---|---|---|
| CD3 | BUV395 | 17A2 | BD | 740268 | 1 |
| CD161 | BUV563 | PK136 | BD | 741233 | 1 |
| CD127 | BUV737 | SB/199 | BD | 612841 | 0.6 |
| CD4 | PAC BLUE | GK1.5 | BioLegend | 100428 | 0.3 |
| PD-1 | BV605 | J43 | BD | 563059 | 0.3 |
| CD25 | BV785 | PC61 | BioLegend | 102051 | 0.5 |
| CD44 | AF488 | IM7 | BioLegend | 103016 | 0.3 |
| KLRG1 | RB613 | 2F1 | BD | 758385 | 1.25 |
| CD69 | RB705 | H1.2F3 | BD | 756896 | 1.25 |
| CD62L | RB780 | MEL-14 | BD | 569209 | 0.3 |
| LAG-3 | PE | C9B7W | BD | 552380 | 0.6 |
| TIM-3 | APC | B8.2C12 | BioLegend | 134008 | 0.6 |
| CD8A | AF700 | 53-6.7 | BD | 557959 | 0.3 |
| L/D | NIR | N/A | Thermo Fisher | L10119 | 0.5 |

TABLE 6
Myeloid panel information (Antibody)

| Marker | Color | Clone | Company | Cat# | ul/100 μL |
|---|---|---|---|---|---|
| CD45 | BUV395 | 30-F11 | Thermo | 363-0451-82 | 0.5 |
| CD11c | BUV496 | N418 | BD | 750450 | 0.125 |
| CD11c | RY703 | N418 | BD | 570292 | 0.125 |
| CD80 | BUV737 | 16-10A1 | BD | 612773 | 1 |
| MHC-II | PAC BLUE | M5/114.15.2 | BioLegend | 107620 | 0.125 |
| Ly6G | BV605 | 1A8 | BioLegend | 127629 | 0.625 |
| CD163 | BV711 | S15049I | BioLegend | 155325 | 0.5 |
| Ly6C | BV785 | HK1.4 | BioLegend | 128041 | 1.25 |
| CD206 | AF488 | C068C2 | BioLegend | 141710 | 0.8 |
| CD86 | RB613 | B7-2 | BD | 759079 | 1 |
| F4/80 | RB780 | T45-2342 | BD | 569223 | 0.25 |
| CD11B | RY703 | M1/70 | BD | 571473 | 0.1 |
| CD11B | RB705 | M1/70 | BD | 755202 | 0.1 |
| PD-L1 | APC | 485 | Thermo Fisher | MA5-46763 | 4 |
| H2Kb Siinfekl | PE | 25-D1.16 | Biolegend | 141604 | 1.25 |
| L/D | NIR | N/A | Thermo Fisher | L101 19 | 0.5 |

TABLE 7
AIM assay information (Antibody)

| Marker | Color | Clone | Company | Cat# | ul/100 μL |
|---|---|---|---|---|---|
| CD40L | FITC | SA047C3 | BD | 157006 | 1 |
| CD25 | PE-CF594 | PC61 | BioLegend | 101920 | 2.5 |
| CD69 | APC | H1.2F3 | Biolegend | 104514 | 3.3 |
| CD3 | AF700 | 17A2 | Biolegend | 100216 | 1 |
| CD8A | APC-H7 | 53-6.7 | BD | 560182 | 2 |
| CD4 | Pac Blue | RM4-4 | BioLegend | 116008 | 1 |
| 4-1BB | PE | 17B5 | BioLegend | 106106 | 1.25 |

TABLE 8
Immunofluorescence information (Antibody)

| Marker | Clone | Company | Cat# | Dilution |
|---|---|---|---|---|
| CD274 (PD-L1) | 2B11D11 | Proteintech | 66248-1-Ig | 1:100 |
| SOX10 | SP267 | Abcam | ab227680 | 1:200 |
| CD3 | 145 2C11 | BD | 553057 | 1:100 |
| CD8 | pAb | Invitrogen | PA5-81344 | 1:1000 |
| PD-1 | J121 | eBioscience | 14-2798-82 | 1:100 |
| Goat anti-mouse | pAB | Invitrogen | A1 1001 | 1:1000 |
| AF488 | | | | |
| Goat anti-rabbit | pAB | Invitrogen | A11012 | 1:1000 |
| AF564 | | | | |
| Goat anti-hamster | pAB | Invitrogen | A78967 | 1:1000 |
| [symbol] | | | | |

[symbol]

indicates data missing or illegible when filed

Table 9: Cox univariate proportional hazards regression analysis of factors influencing overall survival in unresectable Stage III non-small cell lung cancer patients. “COVID mRNA Vaccine” is defined as the receipt of a COVID mRNA vaccination within 100 days of the initiation of immune checkpoint inhibition (ICI). “PDL1” describes tumor proportion score at time of primary tumor biopsy. “Steroid use within 1 month of ICI start” and “Steroid use within 1 month of vaccination” indicated whether or not a patient received a systemic steroid within 1 month before or after immunotherapy start date or vaccination. “Cycle of immunotherapy” described whether the patient was ICI-naïve, had received ICI once before, or had received ICI multiple times, while “Previous Cycles of Systemic Therapy” is very similar but in the context of chemotherapies and other systemic therapies. “EGFR”, “HER2”, “BRAF”, “KRAS”, “ALK”, “PIK3CA”, “MET”, “CDKN2A”, “STK11”, “P53”, “NF”, “RET”, and “ROS1” are descriptions of whether patients had mutations in these genes at the time of tumor biopsy. Performance status quantified by the Eastern Cooperative Oncology Group performance status scale (ECOG), “Age at ICI start”, “BMI”, and comorbidities (heart disease, immunodeficiency, diabetes, other primary, etc.) represent patient characteristics at the start of ICI. “Radiation Prior to ICI” describes whether or not a patient received radiation within 200 days before ICI. “Sex” was described as a categorical variable. “Ethnicity” was described as a categorical variable. “Histology” was described as a categorical variable (Adenocarcinoma, Squamous Cell, etc.). “Immunotherapy Agent” is a categorical variable with the particular type of immune checkpoint immunotherapy used (pembrolizumab, nivolumab, etc.). “Dual Agent ICI” is a binary variable describing whether a patient receives dual ICI (Nivo/lpi) or single ICI alone (Nivo). *p<0.05, **p<0.01, ***p<0.001 unless otherwise noted.

TABLE 9

| Variable | Beta | St. Error | HR | 2.5% CI | 97.5% [symbol] | p [symbol] | P value |
|---|---|---|---|---|---|---|---|
| COVID mRNA vaccination within 100 | −0.8576 | 0.4332 | 0.4242 | 0.1815 | 0.9916 | 0.047 [symbol] | * |
| Stage IIIb (reference: IIIa) | −0.0026 | 0.2767 | 0.9974 | 0.5799 | 1.7154 | 0.992 [symbol] | ns |
| Stage IIIc (reference: IIIa) | 0.0094 | 0.5340 | 1.0095 | 0.3545 | 2.8748 | 0.985 [symbol] | ns |
| Ethnicity American Indian/Alaskan Native | 0.4534 | 1.0122 | 1.5737 | 0.2164 | 11.4425 | 0.654 [symbol] | ns |
| [symbol] | | | | | | | |
| Ethnicity Asian (Reference: White) ++ | [symbol] | 4101.643 [symbol] | 0.0000 | 0.0000 | Inf | 0.996 [symbol] | ns |
| Ethnicity Black (Reference: White) | −0.0862 | 0.4328 | 0.9174 | 0.3928 | 2.1426 | 0.842 [symbol] | ns |
| Ethnicity Other (Reference: White) | 1.6501 | 1.0249 | 5.2075 | 0.6986 | 38.8185 | 0.107 [symbol] | ns |
| Ethnicity Unknown (Reference: White) ++ | [symbol] | 7618.317 [symbol] | 0.0000 | 0.0000 | Inf | 0.998 [symbol] | ns |
| Histology Other (reference: | 0.0765 | 0.5387 | 1.0795 | 0.3756 | 3.103 | 0.887 [symbol] | ns |
| [symbol] | | | | | | | |
| Histology Squamous (reference: | 0.8564 | 0.2781 | 2.3547 | 1.3654 | 4.0609 | 0.002 [symbol] | ** |
| [symbol] | | | | | | | |
| Steroid within 1 month of ICI | 0.3737 | 0.3488 | 1.4531 | 0.7335 | 2.8789 | 0.284 [symbol] | ns |
| Steroid within 1 month of Vaccination | 1.3536 | 1.098 | 3.8713 | 0.4495 | 33.3426 | 0.217 [symbol] | ns |
| Age at ICI Start | 0.0193 | 0.0144 | 1.0195 | 0.9912 | 1.0486 | 0.178 [symbol] | ns |
| ECOG | 0.3625 | 0.1620 | 1.4369 | 1.0460 | 1.9738 | 0.025 [symbol] | * |
| PDL1 (TPS) | −0.0035 | 0.0038 | 0.9965 | 0.9891 | 1.0039 | 0.347 [symbol] | ns |
| Line of Immunotherapy | −0.6079 | 0.8579 | 0.5445 | 0.1013 | 2.9259 | 0.478 [symbol] | ns |
| EGFR | −0.2963 | 0.4695 | 0.7436 | 0.2963 | 1.8663 | 0.528 [symbol] | ns |
| KRAS | 0.0606 | 0.3359 | 1.0625 | 0.5501 | 2.0524 | 0.856 [symbol] | ns |
| BRAF | −0.1683 | 0.7208 | 0.8451 | 0.2058 | 3.4712 | 0.815 [symbol] | ns |
| ALK ++ | [symbol] | 3102.451 [symbol] | 0.0000 | 0.0000 | Inf | 0.996 [symbol] | ns |
| MET | −0.3924 | 0.5944 | 0.6754 | 0.2107 | 2.1653 | 0.509 [symbol] | ns |
| STK11 | 0.6333 | 0.5958 | 1.8838 | 0.5860 | 6.0560 | 0.287 [symbol] | ns |
| ROS1 ++ | [symbol] | 4276.967 [symbol] | 0.0000 | 0.0000 | Inf | 0.996 [symbol] | ns |
| PIK3CA | 0.1203 | 0.5220 | 0.8867 | 0.3188 | 2.4665 | 0.817 [symbol] | ns |
| CDKN2A | 1.0812 | 0.4328 | 2.9481 | 1.2621 | 6.8863 | 0.012 [symbol] | * |
| P53 | −0.0959 | 0.2661 | 0.9086 | 0.5394 | 1.5305 | 0.718 [symbol] | ns |
| NF | 0.3318 | 0.4320 | 1.3935 | 0.5976 | 3.2495 | 0.442 [symbol] | ns |
| RET | 0.3773 | 0.7214 | 1.4584 | 0.3547 | 5.9970 | 0.600 [symbol] | ns |
| Radiation prior to ICI | −0.0086 | 0.2748 | 0.9914 | 0.5785 | 1.6989 | 0.974 [symbol] | ns |
| English as Primary Language (Reference: | 17.0490 | 3925.442 [symbol] | 25368624.19 [symbol] | 0.0000 | Inf | 0.996 [symbol] | ns |
| [symbol] | | | | | | | |
| Heart Disease | −0.0608 | 0.2661 | 0.9410 | 0.5585 | 1.5853 | 0.819 [symbol] | ns |
| Respiratory Condition | 0.5730 | 0.3368 | 1.7736 | 0.9166 | 3.4319 | 0.088 [symbol] | ns |
| Immune Deficiency | 0.1002 | 0.4040 | 1.1054 | 0.5008 | 2.4401 | 0.804 [symbol] | ns |
| Diabetes | 0.4835 | 0.3018 | 1.6217 | 0.8975 | 2.9301 | 0.109 [symbol] | ns |
| CKD | −0.2112 | 0.3821 | 0.8096 | 0.3829 | 1.7120 | 0.580 [symbol] | ns |
| Liver Failure | 0.7460 | 1.0152 | 2.1086 | 0.2883 | 15.4227 | 0.462 [symbol] | ns |
| CNS Disease ++ | [symbol] | 3783.124 [symbol] | 0.0000 | 0.0000 | Inf | 0.996 [symbol] | ns |
| Concurrent Chemotherapy during ICI | 0.2495 | 0.3160 | 1.2833 | 0.6908 | 2.3840 | 0.429 [symbol] | ns |
| Immunotherapy Agent Atezolizumab | 0.1238 | 0.5213 | 1.1318 | 0.4075 | 3.1438 | 0.812 [symbol] | ns |
| [symbol] | | | | | | | |
| Immunotherapy Agent Durvalumab | −0.2283 | 0.3240 | 0.7959 | 0.4217 | 1.5019 | 0.481 [symbol] | ns |
| [symbol] | | | | | | | |
| Immunotherapy Agent Nivolumab | 0.5384 | 0.4633 | 1.7132 | 0.6910 | 4.2475 | 0.245 [symbol] | ns |
| [symbol] | | | | | | | |
| Immunotherapy Agent Nivolumab + | −1.0266 | 1.0359 | 0.3582 | 0.0470 | 2.7282 | 0.321 [symbol] | ns |
| [symbol] | | | | | | | |
| BMI | −0.0259 | 0.0376 | 0.9744 | 0.9051 | 1.0490 | 0.490 [symbol] | ns |
| Gender (Reference: Female) | 0.1366 | 0.2664 | 1.1464 | 0.6801 | 1.9324 | 0.608 [symbol] | ns |
| Previous history of malignancy at ICI Start | −0.2318 | 0.5936 | 0.7931 | 0.2478 | 2.5385 | 0.696 [symbol] | ns |
| Previous cycles of systemic therapy | 0.2040 | 0.1521 | 1.2263 | 0.9102 | 1.6521 | 0.179 [symbol] | ns |
| Treatment Year | −0.0903 | 0.0865 | 0.9136 | 0.7712 | 1.0823 | 0.296 [symbol] | ns |

[symbol]

indicates data missing or illegible when filed

Table 10: Cox multivariate proportional hazards regression analysis of factors influencing overall survival in unresectable Stage Ill non-small cell lung cancer patients. This multivariate analysis included the factors in the univariate analysis that had a significant relationship with overall survival alone in the patient population. See above for descriptions of variables included in analysis. *p<0.05, **p<0.01, ***p<0.001 unless otherwise noted.

TABLE 10

| Variable | Beta | St. Error | adj HR | 2.5% CI | 97.5% CI | p value | P value |
|---|---|---|---|---|---|---|---|
| COVID mRNA | −0.9860 | 0.4453 | 0.3731 | 0.1559 | 0.8930 | 0.0268 | * |
| vaccination within | | | | | | | |
| 100 days of ICI | | | | | | | |
| Histology Other | 0.1067 | 0.6185 | 1.1126 | 0.3310 | 3.7396 | 0.8630 | ns |
| (reference: | | | | | | | |
| Histology Squamous | 0.8644 | 0.2929 | 2.3736 | 1.3370 | 4.2141 | 0.0032 | ** |
| ECOG | 0.3611 | 0.1838 | 1.4349 | 1.0009 | 2.0571 | 0.0494 | * |
| CDKN2A | 1.0148 | 0.4455 | 2.7589 | 1.1521 | 6.6067 | 0.0227 | * |

Table 11: Cox univariate proportional hazards regression analysis of factors influencing overall survival in Stage IV non-small cell lung cancer patients. “COVID mRNA Vaccine” is defined as the receipt of a COVID mRNA vaccination within 100 days of the initiation of immune checkpoint inhibition (ICI). “PDL1” describes tumor proportion score at time of primary tumor biopsy. “Steroid use within 1 month of ICI start” and “Steroid use within 1 month of vaccination” indicated whether or not a patient received a systemic steroid within 1 month before or after immunotherapy start date or vaccination. “Cycle of immunotherapy” described whether the patient was ICI-naïve, had received ICI once before, or had received ICI multiple times, while “Previous Cycles of Systemic Therapy” is very similar but in the context of chemotherapies and other systemic therapies. “EGFR”, “HER2”, “BRAF”, “KRAS”, “ALK”, “PIK3CA”, “MET”, “CDKN2A”, “STK11”, “P53”, “NF”, “RET”, and “ROS1” are descriptions of whether patients had mutations in these genes at the time of tumor biopsy. Performance status quantified by the Eastern Cooperative Oncology Group performance status scale (ECOG), “Age at ICI start”, “BMI”, and comorbidities (heart disease, immunodeficiency, diabetes, other primary, etc.) represent patient characteristics at the start of ICI. “Brain/Liver Mets at ICI Start” describes whether or not a patient had brain or liver mets at the beginning of their relevant ICI treatment. “Sex” was described as a categorical variable. “Ethnicity” was described as a categorical variable. “Histology” was described as a categorical variable (Adenocarcinoma, Squamous Cell, etc.). “Immunotherapy Agent” is a categorical variable with the particular type of immune checkpoint immunotherapy used (pembrolizumab, nivolumab, etc.). “Dual Agent ICI” is a binary variable describing whether a patient receives dual ICI (Nivo/lpi) or single ICI alone (Nivo). *p<0.05, **p<0.01, ***p<0.001 unless otherwise noted.

TABLE 11

| | | St. | | | | | P value |
|---|---|---|---|---|---|---|---|
| Variable | Beta | Error | HR | 2.5% | 97.5% | [symbol] | interpretation |
| COVID mRNA | −0.6063 | 0.1463 | 0.5454 | 0.4094 | 0.7265 | 3.41E−05 | **** |
| vaccination | | | | | | | |
| within 100 days | | | | | | | |
| [symbol] | | | | | | | |
| | 0.1883 | 0.5806 | 1.2072 | 0.3869 | 3.7670 | 0.7457 | ns |
| Ethnicity Asian | 0.3336 | 0.1830 | 1.3961 | 0.9752 | 1.9985 | 0.0683 | ns |
| [symbol] | | | | | | | |
| Ethnicity Black | 0.2169 | 0.1737 | 1.2423 | 0.8839 | 1.7460 | 0.2116 | ns |
| [symbol] | | | | | | | |
| Ethnicity Native | −11.9551 | 1045.5698 | 0.0000 | 0.0000 | Inf | 0.9909 | ns |
| Hawaiian/Pacific | | | | | | | |
| Ethnicity Other | 0.3157 | 0.2490 | 1.3712 | 0.8417 | 2.2336 | 0.2048 | ns |
| [symbol] | | | | | | | |
| Ethnicity [symbol] | −0.2291 | 0.5811 | 0.7953 | 0.2546 | 2.4838 | 0.6934 | ns |
| Histology Other | 0.4205 | 0.3824 | 1.5226 | 0.7196 | 3.2220 | 0.2716 | ns |
| [symbol] | | | | | | | |
| Histology | 0.3814 | 0.2480 | 1.4644 | 0.9007 | 2.3807 | 0.1240 | ns |
| Brain Mets at ICI | 0.2543 | 0.1333 | 1.2896 | 0.9931 | 1.6745 | 0.0564 | ns |
| [symbol] | | | | | | | |
| Liver Mets at ICI | 0.0812 | 0.1816 | 1.0845 | 0.7598 | 1.5481 | 0.6548 | ns |
| [symbol] | | | | | | | |
| Steroid within 1 | 0.3209 | 0.1270 | 1.3784 | 1.0746 | 1.7681 | 0.0115 | * |
| [symbol] | | | | | | | |
| Steroid within 1 | 0.4016 | 0.3850 | 1.4941 | 0.7026 | 3.1774 | 0.2969 | ns |
| [symbol] | | | | | | | |
| Age at ICI Start | 0.0026 | 0.0044 | 1.0026 | 0.9939 | 1.0113 | 0.5620 | ns |
| ECOG | 0.4549 | 0.0640 | 1.5761 | 1.3903 | 1.7867 | 1.17E−12 | **** |
| PDL1 (TPS) | −0.0059 | 0.0015 | 0.9941 | 0.9912 | 0.9969 | 4.88E−05 | **** |
| Line of [symbol] | −0.1969 | 0.1243 | 0.8213 | 0.6437 | 1.0478 | 0.1131 | ns |
| EGFR | 0.1504 | 0.1185 | 1.1623 | 0.9214 | 1.4661 | 0.2044 | ns |
| KRAS | 0.1165 | 0.1023 | 1.1236 | 0.9195 | 1.3729 | 0.2547 | ns |
| HER2ERBB2 | −0.1790 | 0.4502 | 0.8361 | 0.3460 | 2.0205 | 0.6910 | ns |
| BRAF | −0.2258 | 0.2193 | 0.7979 | 0.5192 | 1.2263 | 0.3032 | ns |
| ALK | 0.2453 | 0.2720 | 1.2781 | 0.7500 | 2.1779 | 0.3670 | ns |
| MET | 0.1378 | 0.1289 | 1.1477 | 0.8915 | 1.4776 | 0.2850 | ns |
| STK11 | 0.5429 | 0.1307 | 1.7209 | 1.3321 | 2.2232 | 3.26E−5 | **** |
| ROS1 | −0.0989 | 0.3573 | 0.9058 | 0.4497 | 1.8248 | 0.7820 | ns |
| PIK3CA | 0.1528 | 0.1681 | 1.1651 | 0.8380 | 1.6200 | 0.3634 | ns |
| CDKN2A | 0.1058 | 0.1959 | 1.1116 | 0.7572 | 1.6319 | 0.5892 | ns |
| P53 | 0.1611 | 0.0978 | 1.1748 | 0.9698 | 1.4232 | 0.0996 | ns |
| NF | 0.3283 | 0.1497 | 1.3886 | 1.0356 | 1.8620 | 0.0283 | * |
| RET | 0.6874 | 0.2934 | 1.9886 | 1.1190 | 3.5338 | 0.0191 | * |
| AKT | 0.1534 | 0.7091 | 1.1658 | 0.2904 | 4.6792 | 0.8288 | ns |
| English as Primary | −0.2333 | 0.2295 | 0.7919 | 0.5050 | 1.2418 | 0.3094 | ns |
| Heart Disease | −0.0613 | 0.0980 | 0.9405 | 0.7761 | 1.1397 | 0.5315 | ns |
| Respiratory [symbol] | 0.5368 | 0.1313 | 1.7105 | 1.3224 | 2.2125 | 4.35E−05 | **** |
| Immune [symbol] | −0.0144 | 0.1684 | 0.9857 | 0.7086 | 1.3711 | 0.9318 | ns |
| Diabetes | 0.1348 | 0.1158 | 1.1443 | 0.9119 | 1.4360 | 0.2446 | ns |
| CKD | −0.0602 | 0.1389 | 0.9416 | 0.7171 | 1.2362 | 0.6647 | ns |
| Liver Failure | 0.1504 | 0.3814 | 1.1623 | 0.5504 | 2.4547 | 0.6933 | ns |
| CNS Disease | −0.7091 | 0.7090 | 0.4921 | 0.1226 | 1.9750 | 0.3173 | ns |
| Concurrent [symbol] | 0.1223 | 0.0995 | 1.1301 | 0.9299 | 1.3734 | 0.2189 | ns |
| Immunotherapy | 0.0275 | 0.2260 | 1.0279 | 0.6601 | 1.6006 | 0.9032 | ns |
| Agent [symbol] | | | | | | | |
| [symbol] | 0.1894 | 0.3385 | 1.2086 | 0.6225 | 2.3466 | 0.5757 | ns |
| [symbol] | 0.2472 | 0.1496 | 1.2805 | 0.9551 | 1.7168 | 0.0984 | ns |
| Immunothera [symbol] | 0.0011 | 0.1650 | 1.0011 | 0.7246 | 1.3833 | 0.9945 | ns |
| [symbol] | −13.9764 | 1201.6163 | 0.0000 | 0.0000 | Inf | 0.9907 | ns |
| BMI | 0.0163 | 0.0121 | 1.0165 | 0.9927 | 1.0408 | 0.1753 | ns |
| Gender Male | 0.1806 | 0.0976 | 1.1979 | 0.9894 | 1.4504 | 0.0641 | ns |
| [symbol] | | | | | | | |
| Previous | 0.2574 | 0.3058 | 1.2935 | 0.7104 | 2.3554 | 0.4000 | ns |
| [symbol] | | | | | | | |
| Previous cycles | 0.0870 | 0.0385 | 1.0909 | 1.0115 | 1.1764 | 0.0240 | * |
| [symbol] | | | | | | | |
| Treatment Year | −0.0644 | 0.0293 | 0.9377 | 0.8853 | 0.9931 | 0.0280 | * |

[symbol]

indicates data missing or illegible when filed

Table 12 Cox multivariate proportional hazards regression analysis of factors influencing overall survival in Stage IV non-small cell lung cancer patients. This multivariate analysis included the factors in the univariate analysis that had a significant relationship with overall survival alone in the patient population. See above for descriptions of variables included in analysis. *p<0.05, **p<0.01, ***p<0.001 unless otherwise noted.

TABLE 12

| Variable | Beta | St. Error | HR | 2.5% CI | 97.5% [symbol] | p value | P |
|---|---|---|---|---|---|---|---|
| COVID mRNA vaccination within | −0.6496 | 0.1770 | 0.5223 | 0.3692 | 0.7389 | 0.0002 | *** |
| 100 days of ICI (reference: No | | | | | | | |
| Steroid within 1 month of ICI | 0.3229 | 0.1311 | 1.3811 | 1.0681 | 1.7857 | 0.0138 | * |
| ECOG at ICI Start | 0.4228 | 0.0658 | 1.5263 | 1.3415 | 1.7365 | 1.34E−10 | **** |
| PDL1 | −0.0055 | 0.0016 | 0.9945 | 0.9915 | 0.9975 | 0.0004 | *** |
| STK11 | 0.4428 | 0.1381 | 1.5570 | 1.1877 | 2.0412 | 0.0013 | ** |
| NF | 0.1786 | 0.1563 | 1.1956 | 0.8802 | 1.6240 | 0.2529 | ns |
| RET | 0.5111 | 0.2973 | 1.6672 | 0.9310 | 2.9854 | 0.0855 | ns |
| Respiratory Condition | 0.3460 | 0.1366 | 1.4133 | 1.0813 | 1.8473 | 0.0113 | * |
| Previous cycles of systemic | 0.1003 | 0.0406 | 1.1054 | 1.0210 | 1.1969 | 0.0135 | * |
| Treatment Year | 0.0386 | 0.0386 | 1.0394 | 0.9637 | 1.1210 | 0.3169 | ns |

[symbol]

indicates data missing or illegible when filed

Table 13: Cox univariate proportional hazards regression analysis of factors influencing overall survival in Stage IV melanoma patients receiving their first round of immunotherapy. “COVID mRNA Vaccination” is defined as the receipt of a COVID mRNA vaccination within 100 days of the initiation of immune checkpoint inhibition (ICI). “Steroid use within 1 month of ICI start” and “Steroid use within 1 month of vaccination” indicated whether or not a patient received a systemic steroid within 1 month before or after immunotherapy start date or vaccination. “NRAS”, “BRAF”, “GNA11”, “KIT”, “PTEN”, and “GNAQ” are descriptions of whether patients had mutations in these genes at the time of tumor biopsy. Performance status (ECOG), “Age at ICI start” and comorbidities (heart disease, immunodeficiency, diabetes, other primary, etc.) represent patient characteristics at the start of ICI. “Brain/Liver Mets at ICI Start” describes whether or not a patient had brain or liver mets at the beginning of their relevant ICI treatment. “Sex” was described as a categorical variable. “Ethnicity” was described as a categorical variable. “Immunotherapy Agent” is a categorical variable with the particular type of immune checkpoint immunotherapy used (pembrolizumab, nivolumab, etc.). “Dual Agent ICI” is a binary variable describing whether a patient receives dual ICI (Nivo/lpi) or single ICI alone (Nivo). *p<0.05, **p<0.01, ***p<0.001 unless otherwise noted.

TABLE 13

| | | | | | | | P value |
|---|---|---|---|---|---|---|---|
| Variable | Beta | St. Error | HR | 2.5% CI | 97.5% CI | p value | interpretation |
| COVID mRNA vaccination within 100 | −0.8426 | 0.3016 | 0.4306 | 0.2384 | 0.7777 | 0.0052 | ** |
| days of ICI (relative to No mRNA | | | | | | | |
| Vaccination ever) | | | | | | | |
| Brain Metastasis at ICI Start | 0.4207 | 0.2304 | 1.5230 | 0.9695 | 2.3924 | 0.0679 | ns |
| Liver Metastasis at ICI Start | 0.7483 | 0.2240 | 2.1135 | 1.3626 | 3.2781 | 0.0008 | *** |
| Steroid use within 1 month of | −0.5927 | 0.7693 | 0.5528 | 0.1223 | 2.4971 | 0.4411 | ns |
| Vaccination | | | | | | | |
| Steroid use within 1 month of ICI | 0.6556 | 0.2496 | 1.9262 | 1.1809 | 3.1420 | 0.0086 | ** |
| Initiation | | | | | | | |
| Age at ICI Initiation | 0.0336 | 0.0086 | 1.0342 | 1.0169 | 1.0518 | 9.47E−5 | *** |
| NRAS | −0.1087 | 0.2637 | 0.8970 | 0.5350 | 1.5040 | 0.6801 | ns |
| BRAF | −0.5670 | 0.2307 | 0.5673 | 0.3609 | 0.8915 | 0.0140 | * |
| KIT | 0.4321 | 0.3222 | 1.5405 | 0.8192 | 2.8969 | 0.1799 | ns |
| PTEN | 0.5299 | 0.3357 | 1.6987 | 0.8798 | 3.2796 | 0.1144 | ns |
| GNA11 | 0.6313 | 0.5878 | 1.8801 | 0.5941 | 5.9502 | 0.2828 | ns |
| GNAQ | 1.7676 | 0.5967 | 5.8570 | 1.8188 | 18.8612 | 0.0031 | ** |
| ECOG | 0.5401 | 0.1572 | 1.7162 | 1.2611 | 2.3355 | 0.0006 | *** |
| Gender Male (reference: Female) | 0.0035 | 0.2219 | 1.0035 | 0.6495 | 1.5503 | 0.9875 | ns |
| Ethnicity Asian (Reference: White) | 1.3859 | 0.7198 | 3.9985 | 0.9754 | 16.3914 | 0.0542 | ns |
| Ethnicity Black (Reference: White) | 1.0909 | 0.7179 | 2.9770 | 0.7290 | 12.1568 | 0.1286 | ns |
| Ethnicity Hispanic (Reference: White) | −0.0034 | 0.5126 | 0.9966 | 0.3649 | 2.7218 | 0.9947 | ns |
| Ethnicity Other (Reference: White) | 0.3836 | 1.0073 | 1.4676 | 0.2038 | 10.5681 | 0.7033 | ns |
| Ethnicity Unknown (Reference: White) | −0.5681 | 1.0074 | 0.5666 | 0.0787 | 4.0809 | 0.5728 | ns |
| Immunotherapy Agent Avelumab | 1.3272 | 1.0571 | 3.7705 | 0.4749 | 29.9382 | 0.2093 | ns |
| (Reference: Pembrolizumab) | | | | | | | |
| Immunotherapy Agent Ipilimumab | 2.1307 | 1.0646 | 8.4207 | 1.0452 | 67.8428 | 0.0453 | * |
| (Reference: Pembrolizumab) | | | | | | | |
| Immunotherapy Agent Ipilimumab/ | 0.3758 | 0.3660 | 1.4562 | 0.7107 | 2.9837 | 0.3045 | ns |
| Nivolumab (Reference: Pembrolizumab) | | | | | | | |
| Immunotherapy Agent Nivolumab | 0.3352 | 0.3775 | 1.3982 | 0.6672 | 2.9302 | 0.3746 | ns |
| (Reference: Pembrolizumab) | | | | | | | |
| Immunotherapy Agent Pembrolizumab/ | 0.3696 | 0.6676 | 1.4471 | 0.3911 | 5.3549 | 0.5798 | ns |
| Ipilimumab (Reference: Pembrolizumab) | | | | | | | |
| Concurrent Chemotherapy | 0.6541 | 0.7161 | 1.9233 | 0.4726 | 7.8265 | 0.3610 | ns |
| Heart Disease | 0.3702 | 0.2115 | 1.4480 | 0.9567 | 2.1916 | 0.0800 | ns |
| Respiratory Condition | 0.9011 | 0.3224 | 2.4623 | 1.3089 | 4.6320 | 0.0052 | ** |
| Diabetes | −0.0894 | 0.2327 | 0.9144 | 0.5795 | 1.4429 | 0.7007 | ns |
| CKD | 0.4744 | 0.2636 | 1.6070 | 0.9585 | 2.6943 | 0.0720 | ns |
| Liver Failure | 1.2652 | 0.5147 | 3.5439 | 1.2923 | 9.7182 | 0.0140 | * |
| Immune Deficiency | −0.0346 | 0.2695 | 0.9660 | 0.5697 | 1.6382 | 0.8979 | ns |
| English as Primary Language | −0.4339 | 0.5117 | 0.6480 | 0.2377 | 1.7666 | 0.3965 | ns |
| (Reference: No) | | | | | | | |
| Previous history of malignancy at | −0.2557 | 0.3000 | 0.7743 | 0.4301 | 1.3941 | 0.3940 | ns |
| ICI Start | | | | | | | |
| CNS Disease | 0.2598 | 1.0068 | 1.2967 | 0.1802 | 9.3294 | 0.7964 | ns |
| Treatment Year | −0.4273 | 0.1234 | 0.6523 | 0.5121 | 0.8308 | 0.0005 | *** |

Table 14: Cox multivariate proportional hazards regression analysis of factors influencing overall survival in Stage IV melanoma patients receiving their first round of immunotherapy. This multivariate analysis included the factors in the univariate analysis that had a significant relationship with overall survival alone in the patient population. See above for descriptions of variables included in analysis. *p<0.05, **p<0.01, ***p<0.001 unless otherwise noted.

TABLE 14

| | | St. | | | | | P value |
|---|---|---|---|---|---|---|---|
| Variable | Beta | Error | HR | 2.5% CI | 97.5% CI | p value | interpre [symbol] |
| COVID mRNA vaccination within | −1.0076 | 0.3575 | 0.3651 | 0.1812 | 0.7357 | 0.0048 | ** |
| 100 days of ICI (relative to No | | | | | | | |
| mRNA Vaccination ever) | | | | | | | |
| Liver Metastasis at ICI Start | 0.7319 | 0.2569 | 2.0790 | 1.2565 | 3.4397 | 0.0044 | ** |
| Steroid use within 1 month of | 0.6959 | 0.2923 | 2.0056 | 1.1310 | 3.5566 | 0.0173 | * |
| ICI Initiation | | | | | | | |
| Age at ICI Initiation | 0.0371 | 0.0101 | 1.0378 | 1.0175 | 1.0586 | 0.0002 | *** |
| BRAF | −0.5383 | 0.2559 | 0.5837 | 0.3535 | 0.9640 | 0.0354 | * |
| GNAQ | 1.2506 | 0.7694 | 3.4925 | 0.7730 | 15.7790 | 0.1041 | ns |
| ECOG | 0.4365 | 0.1763 | 1.5472 | 1.0951 | 2.1860 | 0.0133 | * |
| Immunotherapy Agent Avelumab | 0.1541 | 1.1034 | 1.1666\| | 0.1342 | 10.1430 | 0.8889 | ns |
| (Reference: Pembrolizumab) | | | | | | | |
| Immunotherapy Agent | −0.1323 | 1.2936 | 0.8761 | 0.0694 | 11.0564 | 0.9185 | ns |
| Ipilimumab (Reference: | | | | | | | |
| Pembrolizumab) | | | | | | | |
| Immunotherapy Agent | −0.0951 | 0.3996 | 0.9093 | 0.4155 | 1.9902 | 0.8120 | ns |
| Ipilimumab/Nivolumab | | | | | | | |
| Immunotherapy Agent | 0.1160 | 0.3882 | 1.1230 | 0.5247 | 2.4033 | 0.7651 | ns |
| Nivolumab (Reference: | | | | | | | |
| Pembrolizumab) | | | | | | | |
| Immunotherapy Agent | 0.4389 | 0.7013 | 1.5511 | 0.3923 | 6.1322 | 0.5314 | ns |
| Pembrolizumab/Ipilimumab | | | | | | | |
| (Reference: Pembrolizumab) | | | | | | | |
| Respiratory Condition | 0.7733 | 0.3537 | 2.1669 | 1.0833 | 4.3343 | 0.0288 | * |
| Liver Failure | 1.2745 | 0.5375 | 3.5771 | 1.2473 | 10.2585 | 0.0177 | * |
| Treatment Year | −0.2069 | 0.1382 | 0.8131 | 0.6202 | 1.0660 | 0.1343 | ns |

[symbol]

indicates data missing or illegible when filed

Tablo 15: Cox univariate proportional hazards regression analysis of factors influencing progression-free survival in Stage IV melanoma patients receiving their first round of immunotherapy. See above for descriptions of variables included in analysis. *p<0.05, **p<0.01, ***p<0.001 unless otherwise noted.

TABLE 15

| | | St. | | 2.5% | 97.5% | p | P value |
|---|---|---|---|---|---|---|---|
| Variable | Beta | Error | HR | CI | CI | value | interpretati [symbol] |
| COVID mRNA vaccination | −0.4437 | 0.2171 | 0.6416 | 0.4193 | 0.9820 | 0.0410 | * |
| within 100 days of ICI (relative | | | | | | | |
| [symbol] | | | | | | | |
| Brain Metastasis at ICI Start | 0.2023 | 0.1920 | 1.2242 | 0.8403 | 1.7834 | 0.2920 | ns |
| Liver Metastasis at ICI Start | 0.4056 | 0.1940 | 1.5001 | 1.0256 | 2.1942 | 0.0366 | * |
| Steroid use within 1 month of | −0.4324 | 0.4984 | 0.6489 | 0.2443 | 1.7236 | 0.3856 | ns |
| [symbol] | | | | | | | |
| Steroid use within 1 month of | 0.3890 | 0.2048 | 1.4755 | 0.9878 | 2.2042 | 0.0575 | ns |
| ICI [symbol] | | | | | | | |
| Age at ICI Initiation | 0.0080 | 0.0063 | 1.0081 | 0.9958 | 1.0205 | 0.2002 | ns |
| NRAS | −0.0768 | 0.2021 | 0.9261 | 0.6232 | 1.3762 | 0.7039 | ns |
| BRAF | −0.1157 | 0.1704 | 0.8908 | 0.6378 | 1.2440 | 0.4973 | ns |
| KIT | 0.3894 | 0.2654 | 1.4761 | 0.8773 | 2.4835 | 0.1424 | ns |
| PTEN | 0.2188 | 0.2907 | 1.2445 | 0.7040 | 2.2003 | 0.4518 | ns |
| GNA11 | 1.2315 | 0.5133 | 3.4263 | 1.2528 | 9.3709 | 0.0164 | * |
| GNAQ | 0.9556 | 0.5849 | 2.6002 | 0.8263 | 8.1821 | 0.1023 | ns |
| ECOG | 0.4114 | 0.1273 | 1.5089 | 1.1756 | 1.9367 | 0.0012 | ** |
| Gender Male (Reference: | −0.1850 | 0.1724 | 0.8311 | 0.5928 | 1.1652 | 0.2832 | ns |
| [symbol] | | | | | | | |
| Ethnicity Asian (Reference: | 2.2764 | 0.7350 | 9.7414 | 2.3067 | 41.1380 | 0.0020 | ** |
| [symbol] | | | | | | | |
| Ethnicity Black (Reference: | 0.9127 | 0.5869 | 2.4911 | 0.7885 | 7.8697 | 0.1199 | ns |
| [symbol] | | | | | | | |
| Ethnicity Hispanic (Reference: | 0.5003 | 0.3651 | 1.6491 | 0.8063 | 3.3730 | 0.1706 | ns |
| [symbol] | | | | | | | |
| Ethnicity Other (Reference: | 1.4663 | 1.0121 | 4.3333 | 0.5961 | 31.4992 | 0.1474 | ns |
| [symbol] | | | | | | | |
| Ethnicity Unknown (Reference: | −1.6658 | 1.0039 | 0.1890 | 0.0264 | 1.3524 | 0.0971 | ns |
| [symbol] | | | | | | | |
| Immunotherapy Agent Avelumab | 1.2632 | 1.0325 | 3.5368 | 0.4674 | 26.7606 | 0.2212 | ns |
| (Reference: [symbol] | | | | | | | |
| Immunotherapy Agent Ipilimumab | 2.3566 | 1.0461 | 10.5545 | 1.3584 | 82.0057 | 0.0243 | * |
| [symbol] | | | | | | | |
| Immunotherapy Agent Nivolumab | 0.1451 | 0.2740 | 1.1561 | 0.6757 | 1.9780 | 0.5965 | ns |
| [symbol] | | | | | | | |
| Immunotherapy Agent | 0.2351 | 0.2650 | 1.2651 | 0.7525 | 2.1266 | 0.3750 | ns |
| [symbol] | | | | | | | |
| Immunotherapy Agent | 0.3417 | 0.5065 | 1.4074 | 0.5215 | 3.7981 | 0.4999 | ns |
| [symbol] | | | | | | | |
| Concurrent Chemotherapy | 0.3639 | 0.5849 | 1.4389 | 0.4573 | 4.5277 | 0.5339 | ns |
| Heart Disease | 0.3331 | 0.1647 | 1.3953 | 1.0104 | 1.9269 | 0.0431 | * |
| Respiratory Condition | 0.6279 | 0.3021 | 1.8737 | 1.0365 | 3.3871 | 0.0377 | * |
| Diabetes | 0.1943 | 0.1768 | 1.2144 | 0.8588 | 1.7172 | 0.2717 | ns |
| CKD | 0.4658 | 0.2138 | 1.5933 | 1.0479 | 2.4224 | 0.0293 | * |
| Liver Failure | 1.2231 | 0.4599 | 3.3979 | 1.3796 | 8.3688 | 0.0078 | ** |
| Immune Deficiency | 0.4713 | 0.1984 | 1.6021 | 1.0860 | 2.3634 | 0.0175 | * |
| English as Primary Language | −0.2536 | 0.4553 | 0.7760 | 0.3179 | 1.8941 | 0.5776 | ns |
| [symbol] | | | | | | | |
| Previous history of malignancy | −0.4072 | 0.2409 | 0.6655 | 0.4151 | 1.0670 | 0.0909 | ns |
| at [symbol] | | | | | | | |
| CNS Disease | −0.4242 | 1.0039 | 0.6543 | 0.0915 | 4.6804 | 0.6726 | ns |
| Treatment Year | −0.1285 | 0.0848 | 0.8794 | 0.7447 | 1.0385 | 0.1297 | ns |

[symbol]

indicates data missing or illegible when filed

Table 16: Cox multivariate proportional hazards regression analysis of factors influencing progression-free survival in Stage IV melanoma patients receiving their first round of immunotherapy. This multivariate analysis included the factors in the univariate analysis that had a significant relationship with progression-free survival alone in the patient population. See above for descriptions of variables included in analysis. *p<0.05, **p<0.01, ***p<0.001 unless otherwise noted.

TABLE 16

| | | St. | | 2.5% | 97.5% | | P value |
|---|---|---|---|---|---|---|---|
| Variable | Beta | Error | HR | CI | CI | p value | interpret [symbol] |
| COVID mRNA vaccination | −0.4690 | 0.2264 | 0.6256 | 0.4014 | 0.9751 | 0.0383 | * |
| within 100 days of ICI (relative | | | | | | | |
| to No mRNA Vaccination ever) | | | | | | | |
| Liver Mets at ICI Start | 0.2265 | 0.2302 | 1.2541 | 0.7987 | 1.9693 | 0.3253 | ns |
| GNA11 | 0.8993 | 0.5974 | 2.4579 | 0.7621 | 7.9270 | 0.1323 | ns |
| ECOG | 0.3353 | 0.1425 | 1.3983 | 1.0575 | 1.8489 | 0.0187 | * |
| Ethnicity Asian (Reference: White) | 2.2848 | 0.8096 | 9.8240 | 2.0097 | 48.0231 | 0.0048 | ** |
| Ethnicity Black (Reference: White) | 1.0057 | 0.6164 | 2.7339 | 0.8167 | 9.1515 | 0.1028 | ns |
| Ethnicity Hispanic (Reference: White) | 0.6866 | 0.3772 | 1.9869 | 0.9486 | 4.1616 | 0.0687 | ns |
| Ethnicity Other (Reference: White) | 1.3578 | 1.0296 | 3.8877 | 0.5167 | 29.2493 | 0.1872 | ns |
| Ethnicity Unknown (Reference: White) | −1.5399 | 1.0133 | 0.2144 | 0.0294 | 1.5624 | 0.1286 | ns |
| Immunotherapy Agent Avelumab | 0.4880 | 1.0992 | 1.6290 | 0.1889 | 14.0472 | 0.6571 | ns |
| (Reference: Pembrolizumab) | | | | | | | |
| Immunotherapy Agent Ipilimumab | 2.6419 | 1.0790 | 14.0400 | 1.6942 | 116.3530 | 0.0143 | * |
| (Reference: Pembrolizumab) | | | | | | | |
| Immunotherapy Agent Nivolumab | 0.1736 | 0.2866 | 1.1896 | 0.6783 | 2.0862 | 0.5447 | ns |
| (Reference: Pembrolizumab) | | | | | | | |
| Immunotherapy Agent Ipilimumab/ | 0.0608 | 0.2929 | 1.0627 | 0.5986 | 1.8867 | 0.8355 | ns |
| Nivolumab (Reference: Pembrolizumab) | | | | | | | |
| Immunotherapy Agent Pembrolizumab/ | 0.5242 | 0.5282 | 1.6891 | 0.5999 | 4.7557 | 0.3210 | ns |
| Ipilimumab (Reference: Pembrolizumab) | | | | | | | |
| Heart Disease | 0.1894 | 0.1811 | 1.2086 | 0.8475 | 1.7234 | 0.2955 | ns |
| Respiratory Condition | 0.1532 | 0.3593 | 1.1656 | 0.5764 | 2.3571 | 0.6698 | ns |
| Chronic Kidney Disease | 0.2572 | 0.2446 | 1.2933 | 0.8008 | 2.0887 | 0.2930 | ns |
| Liver Failure | 1.2530 | 0.4733 | 3.5007 | 1.3846 | 8.8509 | 0.0081 | ** |
| Immune Deficiency | 0.3799 | 0.2328 | 1.4621 | 0.9265 | 2.3075 | 0.1027 | ns |

[symbol]

indicates data missing or illegible when filed

### REFERENCES

1. Pires da Silva I, Ahmed T, Reijers I L M, Weppler A M, Betof Warner A, Patrinely J R, et al. Ipilimumab alone or ipilimumab plus anti-PD-1 therapy in patients with metastatic melanoma resistant to anti-PD-(L)1 monotherapy: a multicentre, retrospective, cohort study. Lancet Oncol. 2021; 22(6):836-47. Epub 20210511. doi: 10.1016/S1470-2045(21)00097-8. PubMed PMID: 33989557.
2. Geoerger B, Kang H J, Yalon-Oren M, Marshall L V, Vezina C, Pappo A, et al. Pembrolizumab in paediatric patients with advanced melanoma or a PD-L1-positive, advanced, relapsed, or refractory solid tumour or lymphoma (KEYNOTE-051): interim analysis of an open-label, single-arm, phase 1-2 trial. Lancet Oncol. 2020; 21(1):121-33. Epub 20191204. doi: 10.1016/S1470-2045(19)30671-0. PubMed PMID: 31812554.
3. Schachter J, Ribas A, Long G V, Arance A, Grob J J, Mortier L, et al. Pembrolizumab versus ipilimumab for advanced melanoma: final overall survival results of a multicentre, randomised, open-label phase 3 study (KEYNOTE-006). Lancet. 2017; 390(10105):1853-62. Epub 20170816. doi: 10.1016/S0140-6736(17)31601-X. PubMed PMID: 28822576.
4. Borghaei H, Paz-Ares L, Horn L, Spigel D R, Steins M, Ready N E, et al. Nivolumab versus Docetaxel in Advanced Nonsquamous Non-Small-Cell Lung Cancer. N Engl J Med. 2015; 373(17):1627-39. Epub 20150927. doi: 10.1056/NEJMoa1507643. PubMed PMID: 26412456; PMCID: PMC5705936.
5. Carbone D P, Reck M, Paz-Ares L, Creelan B, Horn L, Steins M, et al. First-Line Nivolumab in Stage IV or Recurrent Non-Small-Cell Lung Cancer. N Engl J Med. 2017; 376(25):2415-26. doi: 10.1056/NEJMoa1613493. PubMed PMID: 28636851; PMCID: PMC6487310.
6. Fehrenbacher L, Spira A, Ballinger M, Kowanetz M, Vansteenkiste J, Mazieres J, et al. Atezolizumab versus docetaxel for patients with previously treated non-small-cell lung cancer (POPLAR): a multicentre, open-label, phase 2 randomised controlled trial. Lancet. 2016; 387(10030):1837-46. Epub 20160310. doi: 10.1016/S0140-6736(16)00587-0. PubMed PMID: 26970723.
7. Garon E B, Rizvi N A, Hui R, Leighl N, Balmanoukian A S, Eder J P, et al. Pembrolizumab for the treatment of non-small-cell lung cancer. N Engl J Med. 2015; 372(21):2018-28. Epub 20150419. doi: 10.1056/NEJMoa1501824. PubMed PMID: 25891174.
8. Herbst R S, Baas P, Kim D W, Felip E, Perez-Gracia J L, Han J Y, et al. Pembrolizumab versus docetaxel for previously treated, PD-L1-positive, advanced non-small-cell lung cancer (KEYNOTE-010): a randomised controlled trial. Lancet. 2016; 387 (10027): 1540-50. Epub 20151219. doi: 10.1016/S0140-6736 (15) 01281-7. PubMed PMID: 26712084.
9. Rittmeyer A, Barlesi F, Waterkamp D, Park K, Ciardiello F, von Pawel J, et al. Atezolizumab versus docetaxel in patients with previously treated non-small-cell lung cancer (OAK): a phase 3, open-label, multicentre randomised controlled trial. Lancet. 2017; 389 (10066): 255-65. Epub 20161213. doi: 10.1016/S0140-6736(16)32517-X. PubMed PMID: 27979383; PMCID: PMC6886121.
10. Sayour E J, Grippin A, De Leon G, Stover B, Rahman M, Karachi A, et al. Personalized Tumor RNA Loaded Lipid-Nanoparticles Prime the Systemic and Intratumoral Milieu for Response to Cancer Immunotherapy. Nano Lett. 2018; 18(10):6195-206. Epub 20180927. doi: 10.1021/acs.nanolett.8b02179. PubMed PMID: 30259750; PMCID: PMC6597257.
11. Sayour E J, De Leon G, Pham C, Grippin A, Kemeny H, Chua J, et al. Systemic activation of antigen presenting cells via RNA-loaded nanoparticles. Oncolmmunology. 2016:00-doi: 10.1080/2162402X.2016.1256527.
12. Jenkins R W, Barbie D A, Flaherty K T. Mechanisms of resistance to immune checkpoint inhibitors. British Journal of Cancer. 2018; 118 (1): 9-16. doi: 10.1038/bjc.2017.434.
13. Mendez-Gomez H R, DeVries A, Castillo P, von Roemeling C, Qdaisat S, Stover B D, et al. RNA aggregates harness the danger response for potent cancer immunotherapy. Cell. 2024; 187 (10): 2521-35 e21. Epub 20240501. doi: 10.1016/j.cell.2024.04.003. PubMed PMID: 38697107.
14. Tahtinen S, Tong A J, Himmels P, Oh J, Paler-Martinez A, Kim L, et al. IL-1 and IL-1ra are key regulators of the inflammatory response to RNA vaccines. Nat Immunol. 2022; 23(4):532-42. Epub 20220324. doi: 10.1038/s41590-022-01160-y. PubMed PMID: 35332327.
15. Sousa L G, McGrail D J, Li K, Marques-Piubelli M L, Gonzalez C, Dai H, et al. Spontaneous tumor regression following COVID-19 vaccination. J Immunother Cancer. 2022; 10(3). doi: 10.1136/jitc-2021-004371. PubMed PMID: 35241495; PMCID: PMC8896046.
16. Bafaloukos D, Petraki K, Bousmpoukea A, Chatzichristou E, Pieris I, Koutserimpas C, et al. Therapeutic Effect of mRNA SARS-CoV-2 Vaccine on Melanoma Skin Metastases. Vaccines-Basel. 2022; 10(4). doi: ARTN 525
10.3390/vaccines10040525. PubMed PMID: WOS: 000785456700001.
17. Khalili J S, Liu S, Rodriguez-Cruz T G, Whittington M, Wardell S, Liu C, et al. Oncogenic BRAF(V600E) promotes stromal cell-mediated immunosuppression via induction of interleukin-1 in melanoma. Clin Cancer Res. 2012; 18(19):5329-40. Epub 20120731. doi: 10.1158/1078-0432.CCR-12-1632. PubMed PMID: 22850568; PMCID: PMC3463754.
18. Poloni C, Schonhofer C, Ivison S, Levings M K, Steiner T S, Cook L. T-cell activation-induced marker assays in health and disease. Immunol Cell Biol. 2023; 101(6):491-503. Epub 20230321. doi: 10.1111/imcb. 12636. PubMed PMID: 36825901; PMCID: PMC10952637.
19. Acar R, Paydas S, Yildirim M, Kilicarslan E, Sahin U, Dogan A, et al. Treatment options in primary mediastinal B cell lymphoma patients, retrospective multicentric analysis; a Turkish oncology group study. J Cancer Res Ther. 2023; 19 (Supplement): S138-S44. doi: 10.4103/jcrt.jcrt_355_22. PubMed PMID: 37147993.
20. Munoz F M, Sher L D, Sabharwal C, Gurtman A, Xu X, Kitchin N, et al. Evaluation of BNT162b2 Covid-19 Vaccine in Children Younger than 5 Years of Age. N Engl J Med. 2023; 388 (7): 621-34. doi: 10.1056/NEJMoa2211031. PubMed PMID: 36791162; PMCID: PMC9947923.
21. Simoes E A F, Klein N P, Sabharwal C, Gurtman A, Kitchin N, Ukkonen B, et al. Immunogenicity and Safety of a Third COVID-19 BNT162b2 mRNA Vaccine Dose in 5- to 11-Year Olds. J Pediatric Infect Dis Soc. 2023; 12 (4): 234-8. doi: 10.1093/jpids/piad015. PubMed PMID: 36929216; PMCID: PMC10146923.
22. Verbeke R, Lentacker I, De Smedt S C, Dewitte H. The dawn of mRNA vaccines: The COVID-19 case. J Control Release. 2021; 333:511-20. Epub 20210330. doi: 10.1016/j.jconrel.2021.03.043. PubMed PMID: 33798667; PMCID: PMC8008785.
23. Gainor J F, Patel M R, Weber J S, Gutierrez M, Bauman J E, Clarke J M, et al. T Cell Responses to Individualized Neoantigen Therapy mRNA-4157 (V940) Alone or in Combination With Pembrolizumab in the Phase 1 KEYNOTE-603 Study. Cancer Discov. 2024. Epub 20240808. doi: 10.1158/2159-8290.CD-24-0158. PubMed PMID: 39115419.
24. Weber J S, Carlino M S, Khattak A, Meniawy T, Ansstas G, Taylor M H, et al. Individualised neoantigen therapy mRNA-4157 (V940) plus pembrolizumab versus pembrolizumab monotherapy in resected melanoma (KEYNOTE-942): a randomised, phase 2b study. Lancet. 2024; 403(10427):632-44. Epub 20240118. doi: 10.1016/S0140-6736(23)02268-7. PubMed PMID: 38246194.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. If aspects of the invention are described as “comprising” a feature, embodiments also are contemplated “consisting of” or “consisting essentially of” the feature.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein. Other than in the operating examples, or where otherwise indicated, all numbers expressing quantities of ingredients or reaction conditions used herein should be understood as modified in all instances by the term “about” as that term would be interpreted by the person skilled in the relevant art.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

What is claimed is:

1. A method of increasing sensitivity of a tumor to treatment with an immune checkpoint inhibitor (ICI), the method comprising (a) administering to a subject in need thereof a composition comprising a lipid nanoparticle comprising mRNA encoding SARS-CoV-2 Spike protein and (b) administering an ICI to the subject.

2. The method of claim 1, wherein the composition comprising a lipid nanoparticle comprising mRNA encoding SARS-CoV-2 Spike protein is administered within 100 days of administration of the ICI.

3. The method of claim 1, wherein the ICI is a PD-L1 inhibitor, a PD-1 inhibitor, or a CTLA4 inhibitor, such as an anti-PD-L1 antibody, anti-PD-1 antibody, or anti-CTLA4 antibody.

4. The method of claim 1, wherein the subject is suffering from non-small cell lung cancer or melanoma.

5. The method of claim 1, wherein the lipid nanoparticle comprises mRNA, SM-102, polyethylene glycol [PEG] 2000 dimyristoyl glycerol [DMG], cholesterol, and 1,2-distearoyl-sn-glycero-3-phosphocholine [DSPC].

6. The method of claim 1, wherein the lipid nanoparticle comprises mRNA, ((4-hydroxybutyl)azanediyl)bis(hexane-6,1-diyl)bis(2-hexyldecanoate), 2-(polyethylene glycol 2000)-N,N-ditetradecylacetamide, 1,2-distearoyl-sn-glycero-3-phosphocholine, and cholesterol.

7. The method of claim 1, wherein the composition comprising a lipid nanoparticle comprising mRNA encoding SARS-CoV-2 Spike protein is administered within 30 days of administration of the ICI.

8. The method of claim 1, wherein the composition comprising a lipid nanoparticle comprising mRNA encoding SARS-CoV-2 Spike protein is administered ipsilaterally with respect to the tumor.

9. A method of increasing sensitivity of a tumor to treatment with an immune checkpoint inhibitor (ICI), the method comprising (a) administering to a subject in need thereof a composition comprising a lipid nanoparticle comprising mRNA encoding a viral antigen and (b) administering an ICI to the subject, wherein the composition comprising a lipid nanoparticle comprising mRNA encoding a non-tumor antigen is administered within 100 days of administration of the ICI.

10. The method of claim 9, wherein the composition comprising a lipid nanoparticle comprising mRNA encoding a viral antigen is administered within 30 days of administration of the ICI.

11. The method of claim 9, wherein the viral antigen comprises SARS-CoV-2 Spike protein or pp65.

12. The method of claim 9, wherein the ICI is a PD-L1 inhibitor, a PD-1 inhibitor, or a CTLA4 inhibitor, such as an anti-PD-L1 antibody, anti-PD-1 antibody, or anti-CTLA4 antibody.

13. The method of claim 9, wherein the subject is suffering from non-small cell lung cancer or melanoma.

14. The method of claim 9, wherein the lipid nanoparticle comprises (a) mRNA, SM-102, polyethylene glycol [PEG] 2000 dimyristoyl glycerol [DMG], cholesterol, and 1,2-distearoyl-sn-glycero-3-phosphocholine [DSPC] or (b) mRNA, ((4-hydroxybutyl)azanediyl)bis(hexane-6,1-diyl)bis(2-hexyldecanoate), 2-(polyethylene glycol 2000)-N,N-ditetradecylacetamide, 1,2-distearoyl-sn-glycero-3-phosphocholine, and cholesterol.

15. The method of claim 1, wherein the composition comprising a lipid nanoparticle comprising mRNA encoding SARS-CoV-2 Spike protein is administered ipsilaterally with respect to the tumor.

16. A method of treating a subject with an immune checkpoint inhibitor (ICI)-resistant tumor, the method comprising (a) administering to a subject in need thereof a composition comprising a lipid nanoparticle comprising mRNA encoding SARS-CoV-2 Spike protein and (b) administering an ICI to the subject.

17. The method of claim 1, wherein the composition comprising a lipid nanoparticle comprising mRNA encoding SARS-CoV-2 Spike protein is administered within 100 days of administration of the ICI.

18. The method of claim 1, wherein the ICI is a PD-L1 inhibitor, a PD-1 inhibitor, or a CTLA4 inhibitor, such as an anti-PD-L1 antibody, anti-PD-1 antibody, or anti-CTLA4 antibody.

19. The method of claim 1, wherein the lipid nanoparticle comprises (a) mRNA, SM-102, polyethylene glycol [PEG] 2000 dimyristoyl glycerol [DMG], cholesterol, and 1,2-distearoyl-sn-glycero-3-phosphocholine [DSPC] or (b) mRNA, ((4-hydroxybutyl)azanediyl)bis(hexane-6,1-diyl)bis(2-hexyldecanoate), 2-(polyethylene glycol 2000)-N,N-ditetradecylacetamide, 1,2-distearoyl-sn-glycero-3-phosphocholine, and cholesterol.

20. The method of claim 1, wherein the composition comprising a lipid nanoparticle comprising mRNA encoding SARS-CoV-2 Spike protein is administered ipsilaterally with respect to the tumor.
